(19) United States
(12) Patent Application Publication
(10) Pub. No.: US 2025/0108070 A1
(43) Pub. Date: Apr. 3, 2025

(54) METHOD FOR REPROGRAMMING CD8+ T CELLS TO ENHANCE THEIR THERAPEUTIC POTENTIAL AND APPLICATIONS THEREOF

(71) Applicant: INSTITUT PASTEUR, PARIS CEDEX 15 (FR)

(72) Inventors: Asier SAEZ CIRION, PARIS (FR); Federico PERDOMO CELIS, Bogota (CO); Caroline PEREIRA BITTENCOURT PASSAES, PARIS (FR); Michaela MULLER-TRUTWIN, PARIS (FR)

(21) Appl. No.: 18/730,157

(22) PCT Filed: Jan. 20, 2023
(86) PCT No.: PCT/EP2023/051448

§ 371 Date: Jul. 18, 2024

Related U.S. Application Data
(60) Provisional application No. 63/301,532, filed on Jan. 21, 2022.

(51) Int. Cl. A61K 35/17; A61K 40/11; C12N 5/0783; G01N 33/50

(52) U.S. Cl. CPC A61K 35/17; A61K 40/11; C12N 5/0636; G01N 33/505; C12N 2501/2315; C12N 2501/727

(57) ABSTRACT

The invention relates to a method for reprogramming CD8+ T cells to enhance their therapeutic potential and its applications, in particular in adoptive T cell therapy for the treatment of infectious diseases and cancer.

### FIELD OF THE INVENTION

The invention pertains to the field of immunotherapy. The invention relates to a method for reprogramming CD8+ T cells to enhance their therapeutic potential and its applications, in particular in adoptive T cell therapy for the treatment of infectious diseases and cancer.

### BACKGROUND OF THE INVENTION

For activation and differentiation, CD8⁺ T cells require T cell receptor (TCR) signals provided by peptide/major histocompatibility complex class I, together with costimulation, and cytokines. The amount and duration of these signals influence the priming and fate of memory CD8⁺ T cells (1). Human immunodeficiency virus type 1 (HIV-1) infection is an example of the different outcomes a CD8⁺ T cell can reach according to the quality of the priming received early after infection. For instance, HIV-1 controllers (HICs), who represent less than 0.5% of people living with HIV-1, control viremia for long periods in the absence of antiretroviral therapy, and such control is associated with strong HIV-specific CD8⁺ T cell responses (2,3). Compelling studies indicate that HIV-specific CD8⁺ T cells from HICs exhibit stemness properties, high survival, proliferative potential, and polyfunctionality upon antigen stimulation (4-7). Recently, it has been demonstrated that HIV-specific central memory CD8⁺ T cells from HICs have a stem-like transcriptional program, characterized by enhanced polyfunctionality and survival, compared with cells from non-controllers (8). Moreover, it was previously reported that early in primary simian immune deficiency virus (SIV) infection there is a divergence in the memory compartment of SIV-specific CD8⁺ T cells of macaques that naturally controlled or did not control infection later in the chronic phase. In this animal model, SIV controllers developed stem-like SIV-specific CD8⁺ T cells, which preceded the optimal maturation of the CD8⁺ T cell response (i.e., acquisition of SIV-suppressive properties) during infection (9). Contrary to cells from HICs or SIV controllers, virus-specific memory CD8⁺ T cells from non-controllers exhibit an effector-like and exhausted profile, limited survival capacity, and poor antiviral potential throughout infection (8-10). Overall, these observations are consistent with tuned priming of virus-specific CD8⁺ T cells early after infection in natural controllers, promoting long-lived stem-like memory responses that mature throughout infection, whereas there is a skewed or non-regulated CD8⁺ T cell priming in non-controllers that promotes short-lived effector-like responses.

In part, the stem-like profile of memory CD8⁺ T cells is mediated by the transcription factor TCF-1, a downstream component of the Wnt/β-catenin pathway (11). As such, TCF-1 regulates stemness properties of CD8⁺ T cells, including longevity, self-renewal, and the potential to differentiate into multiple subsets (12). Evidence from chronic infection models indicates that stem-like memory TCF-1V CD8⁺ T cells have an enhanced capacity to contain the infection and respond to secondary viral challenges (13-15). Fittingly, it was demonstrated that virus-specific CD8⁺ T cells from HIV-1 and SIV controllers exhibit higher levels of TCF-1 compared with cells from non-controllers (7,9,16), further suggesting that stem-like memory cells exert a protective role in HIV-1/SIV infections.

Several metabolic cues, such as the mammalian target of rapamycin complex (mTORC) 1 and 2, also regulate CD8⁺ T cell fate, longevity, and effector functions (17). Constitutive activation of the mTORC1 pathway results in terminal differentiation of CD8⁺ T cells and lack of long-term memory, whereas inhibition of mTORC2 leads to excessive activation and cell death (18). Accordingly, mTORC1 inhibition with rapamycin increases the number, quality, and survival of stem-like memory CD8⁺ T cells (19,20). In line with those data, it was previously shown that HIV-specific memory CD8⁺ T cells from HICs maintain metabolic plasticity and preferential activation of the mTORC2 pathway, whereas cells from non-controllers are more dependent on glycolysis and mTORC1 (8). Thus, the ability to use diverse metabolic resources, as well as the suitable engagement of mTORC1 and 2 pathways, contribute to the enhanced functionality and longevity of memory HIV-specific CD8⁺ T cells from HICs.

It was shown recently that metabolic reprogramming with IL-15 increases mitochondrial respiration in CD8⁺ T cells from non-controllers, contributing to improve their viral-suppressive capacity ex vivo (8).

The serine/threonine glycogen synthase kinase-3 (GSK-3 or GSK3) is a conserved signaling molecule with essential roles in diverse biological processes including metabolism, gene expression, cell fate determination, proliferation, and survival. GSK3 exists in two isoforms, termed a and p, that are highly related and largely redundant. GSK3 is bidirectionally regulated by phosphorylation at Y216/276 (activating) and S21/9 (inactivating). Exemplary amino acid sequences for human GSK3 alpha and beta proteins include UniPRot accession numbers P49840 and P49841, respectively. GSK3 is a key player in the phosphatidylinositol (PtdIns)-3-kinase-dependent pathway that is triggered by insulin and growth factors and the Wnt/β-catenin signaling pathway. GSK3 phosphorylates β-catenin leading to the subsequent degradation of this molecule. GSK3 is also directly involved in the degradation of rictor an essential component of mTOR complex 2 (48). GSK3 also phosphorylates and activates Raptor at Ser859, which in turns allows the activation of mTORC1 (Stretton et al., Biochem. J., 2015, 470, 207-221). Aberrant GSK3 activity has been associated with numerous human diseases and many GSK3 inhibitors have been developed, some of which are used in human therapy or currently tested in clinical trials (Review in Front. Mol. Neurosci., 31 Oct. 2011, doi:10.3389).

Antigen-specific CD8+ T cells and in particular the subset of long-lived memory CD8+ T cells, play an important role in the control of diseases such as infectious diseases and cancer. Consequently, to improve the treatment of these diseases, there exists a need for the development of methods for reprogramming CD8+ T cells in pathological conditions to improve their therapeutic potential. The invention fulfils this need.

### BRIEF SUMMARY OF THE INVENTION

The invention encompasses a method for reprogramming CD8+ T cells to enhance their therapeutic potential and its various uses, in particular in methods of adoptive T cell therapy of cancer and infectious diseases. The invention encompasses also a screening method for inducers of reprogramming of CD8+ T cells.

In the present study, the inventors have targeted the Wnt/TCF-1 and mTORC signaling pathways using a GSK3 inhibitor to reprogram CD8+ T cells. Inhibition of GSK3, herein referred to as CD8+ T cell reprogramming, promotes a stem-like memory phenotype or modulates (promotes or represses) effector-like phenotype in CD8⁺ T cells. Reprogramming does not require any further stimulation of the CD8+ T cells. The reprogrammed CD8+ T cells maintain their stem-like memory phenotype upon activation. Furthermore, the reprogrammed CD8+ T cells have an enhanced potential for immunotherapy or cancer and infectious diseases as evidenced by their superior response to antigen, enhanced survival, polyfunctionality, proliferative capacity, metabolic plasticity, less mTORC1-dependency, and improved response to γ-chain cytokines. Then, the inventors have evaluated the potential of CD8⁺ T cell reprogramming for inducing stem-like properties in cells derived from HIV-1 non-controller individuals, via the manipulation of the TCF-1 and mTORC pathways. They found that, after reprogramming, virus-specific CD8⁺ T cells from non-controllers acquire phenotypic, transcriptional, metabolic, and functional attributes associated with natural control of HIV-1 infection which help to improve the response to immunomodulators. CD8⁺ T cell reprogramming could be used to potentiate the therapeutic efficacy of this cell population in adoptive transfer strategies, as well as the effect of other immunotherapies, in the search for an HIV-1 cure or remission or development of therapeutic vaccines. In addition, the benefits of T cell reprogramming could be extended to the context of other chronic viral infections or tumors where antigen-specific CD8⁺ T cells have a skewed and dysfunctional profile.

One aspect of the invention relates to a method for reprogramming CD8+ T cells, comprising administering a glycogen synthase kinase-3 (GSK3) inhibitor, in vitro, to a population of human CD8+ T cells, wherein the GSK3 inhibitor induces reprogrammed CD8+ T cells having enhanced stemness and functional properties or a modulation (enhancement or repression) of effector cells compared to non-reprogrammed CD8+ T cells.

In some embodiments, the CD8+ T cells are from peripheral blood. In some embodiments, the CD8+ T cells of the population are resting.

In some embodiments, the GSK3 inhibitor is selected from the group consisting of: small metal cations; small-molecule ATP-competitive inhibitors; small-molecule non-ATP competitive inhibitors; substrate-competitive peptide inhibitors; siRNAs; miRNAs, ribozymes; epigenome editing enzyme complexes; antagonist peptides; antagonist antibodies; antagonist aptamers, and combinations thereof.

In some embodiments, the GSK3 inhibitor is selected from the group consisting of: Anilinomaleimides, ions, Pyrroloazepines, Flavones, Benzazepinones, Bis-Indoles, in particular Indirubins, Pyrrolopyrazines, Aminopyrimidines, Oxindoles, Thiazoles, Bisindolylmaleimides, Phenylaminopyrimidines, Pyrrolopyrimidines, Pyrazines, Thiadiazolidinones and Oxazole-carboxamides, and combinations thereof. In some preferred embodiments, the GSK3 inhibitor is selected from the group consisting of: SB-415286, Lithium carbonate, 10Z-Hymenialdisine, Flavopiridol hydrochloride hydrate, Alsterpaullone, 6-bromoindirubin-3′-oxime and 6-bromoindirubin-3′-acetoxime, Aloisine A, CHIR 98014, SU9516, AR-A014418, Staurosporine, GF 109203X, CGP 60474, TWS119, AZD2858, NP031112, SB-216763, PF-04802367, functional analogs or derivatives thereof, and combinations thereof.

In some particular embodiments, the GSK3 inhibitor is an indirubin of formula (I) or a pharmaceutically acceptable salt thereof:

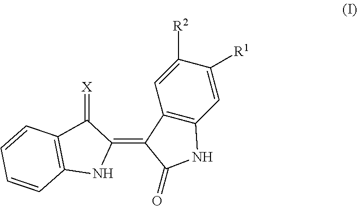

wherein
R¹ is a halogen, or a vinyl group (—CH═CH₂);
R² is selected from the group consisting of H, halogen, amino, nitro and C₁₋₅ alkyl; and
X is O or N—OR³, wherein R³ is selected from the group consisting of H, -(A)-R⁴, —C(O)R⁵ and —C(O)N(R⁶, R⁷), with
A being a non-substituted C₁₋₅ alkylene group or a C₁₋₅ alkylene group substituted by one or several A¹ groups, A¹ being halogen, OH, OR⁸ or NH₂, R⁸ being a C₁₋₅ alkyl;
R⁴ being selected from the group consisting of H, halogen, OH and N(R⁶, R⁷);
R⁵ being a C₁₋₅ alkyl;
R⁶ and R⁷, identical or different, being a non-substituted C₁₋₅ alkyl or a C₁₋₅ alkyl substituted by A¹ such as above defined, or R⁶ and R⁷ are part of a cycle with 5 or 6 elements, optionally comprising another heteroatom such as O or N.

Preferably, the GSK3 inhibitor is 6-bromoindirubin-3′-oxime; 6-bromoindirubin-3′-acetoxime, or a functional analog or derivative thereof, or combination thereof.

In some particular embodiments, the GSK3 inhibitor for CD8+ T cell reprogramming induces CD8+ T cells having enhanced stemness and functional properties and is selected from indirubins, in particular indirubin of formula (I) as disclosed herein. In some preferred embodiments, the GSK3 inhibitor is selected from the group consisting of: 6-bromoindirubin-3′-oxime, 6-bromoindirubin-3′-acetoxime, 6-bromo-indirubin, 6-bromo-indirubin-3′methoxime, 6-chloro-indirubin, 6-chloro-indirubin-3′-oxime, 6-chloro-indirubin-3′-acetoxime, 6-iodo-indirubin, 6-iodo-indirubin-3′-oxime, 6-iodo-indirubin-3′-acetoxime, 6-vinylindirubin-3′-oxime, 6-vinylindirubin-3′-acetoxime, 6-fluoroindirubin-3′-oxime, 6-fluoroindirubin-3′-acetoxime, 6-bromo-5-methylindirubin, 6-bromo-5-methylindirubin-3′-oxime, 6-bromo-5-methylindirubin-3′-acetoxime, 5,6-dichloroindirubin, 5,6-dichloroindirubin-3′-oxime, 5,6-dichloroindirubin-3′-acetoxime, 6-bromo-5-nitroindirubin, 6-bromo-5-nitroindirubin-3′-oxime, 6-bromo-5-nitroindirubin-3′-acetoxime, 6-bromo-5-aminoindirubin, 6-bromo-5-aminoindirubin-3′-oxime, 5,6-dibromoindirubin, 6-bromoindirubin-3′-[O-(2-bromoethyl)-oxime], 6-bromoindirubin-3′-[O-(2-hydroxyethyl)-oxime], 6-bromoindirubin-3′-[O-(2,3-dihydroxypropyl)-oxime], 6-bromoindirubin-3′-[0-(N,N-diethylcarbamyl)-oxime], 6-bromoindirubin-3′-[O-(2-dimethylaminoethyl)-oxime], 6-bromoindirubin-3′-[O-(2-diethylaminoethyl)-oxime], 6-bromoindirubin-3′-[O-(2-pyrrolidin-1-ylethyl)oxime], 6-bromoindirubin-3′-[O-(2-morpholin-1-ylethyl)oxime], 6-bromoindirubin-3′-[O-(2-(N,N-(2-hydroxyethyl)aminoethyl)oxime], 6-bromoindirubin-3′-(0-{2-[N-methyl, N-(2,3-dihydroxypropyl)amino]ethyl}oxime], 6-bromoindirubin-3′-[0-(2-piperazine-1-ylethyl)oxime], 6-bromoindirubin-3′-(0-[2-(4-methyl-piperazin-1-yl)ethyl]oxime), 6-bromoindirubin-3′-(O-{2-[4-(2-hydroxyethyl)piperazin-1-yl]ethyl}oxime), 6-bromoindirubin-3′-(O-{2-[4-(2-methoxyethyl)piperazin-1-yl]ethyl}oxime), 6-bromoindirubin-3′-[O-(2-(4-[2-(2-hydroxyethoxy)-ethyl]piperazin-1-yl)ethyl)oxime], 6-bromoindirubin-3′-[O-(2-dimethylaminoethyl)oxime]hydrochloride, 6-bromoindirubin-3′-[O-(2-diethylaminoethyl)oxime]hydrochloride, 6-bromoindirubin-3′-[0-(2-pyrrolidin-1-ylethyl)oxime]hydrochloride, 6-bromoindirubin-3′-[O-(2-morpholin-1-ylethyl)oxime]hydrochloride, 6-bromoindirubin-3′-[O-(2-(N,N-(2-hydroxyethyl)aminoethyl)oxime], 6-bromoindirubin-3′-(O-{2-[N-methyl, N-(2,3-dihydroxypropyl)amino]ethyl}oxime]hydrochloride, 6-bromoindirubin-3′-[O-(2-piperazine-1-ylethyl)oxime]dihydrochloride, 6-bromoindirubin-3′-{0-[2-(4-methylpiperazin-1-yl)ethyl]oxime}dihydrochloride, 6-bromoindirubin-3′-(O-{2-[4-(2-hydroxyethyl)piperazin-1-yl]ethyl}oxime)dihydrochloride, 6-bromoindirubin-3′-(O-{2-[4-(2-methoxyethyl)piperazin-1-yl]ethyl}oxime)dihydrochloride and 6-bromoindirubin-3′-[O-(2-{4-[2-(2-hydroxyethoxy)-ethyl]piperazin-1-yl}ethyl)oxime]dihydrochloride; preferably 6-bromoindirubin-3′-oxime and 6-bromoindirubin-3′-acetoxime.

In some particular embodiments, the GSK3 inhibitor for CD8+ T cell reprogramming enhances effector CD8+ T cells and is selected from the group consisting of: Anilinomaleimides; Benzazepinones; Aminopyrimidines, Oxindoles; Pyrazines and Oxazole-carboxamides; preferably selected from the group consisting of: Benzazepinones; Oxindoles; Pyrazines and Oxazole-carboxamides. In some preferred embodiments, the GSK3 inhibitor is selected from the group consisting of: Alsterpaullone, CHIR 98014, SU9516, AZD2858, SB-216763, PF-04802367, functional analogs or derivatives thereof, and combinations thereof; preferably Alsterpaullone, SU9516, AZD2858, PF-04802367, functional analogs or derivatives thereof, and combinations thereof.

In some particular embodiments, the GSK3 inhibitor for CD8+ T cell reprogramming represses effector CD8+ T cells and is selected from the group consisting of: Anilinomaleimides and Bisindolylmaleimides; Pyrroloazepines; Flavones; Pyrrolopyrazines; Pyrrolopyrimidines and Phenylaminopyrimidines; Thiazoles; and Thiadiazolidinones. In some preferred embodiments, the GSK3 inhibitor is selected from the group consisting of: SB-415286, 10Z-Hymenialdisine, Flavopiridol hydrochloride hydrate, Aloisine A, AR-A014418, Staurosporine, GF 109203X, CGP 60474, TWS119, NP031112, functional analogs or derivatives thereof, and combinations thereof.

In some embodiments, the GSK3 inhibitor is at a concentration of from 1 to 5 μM. In some embodiments, the GSK3 inhibitor, the GSK3 inhibitor is contacted with the population of CD8+ T cells for 6 to 16 hours.

In some embodiments, the reprogrammed CD8+ T cells have enhanced survival, polyfunctionality, proliferation capacity, metabolic plasticity, response to antigen, response to γ-chain cytokines, response to immune check-point modulators, cytotoxic effect, and/or less mTORC1-dependency, compared to non-reprogrammed CD8+ T cells.

In some preferred embodiments, the reprogrammed CD8+ T cells have enhanced expression level of TCF-1 in the absence of further stimulation; preferably further having enhanced expression levels of CCR7 and CD27 in the absence of further stimulation.

In some preferred embodiments, the reprogrammed CD8+ T cells have enhanced expression levels of TCF-1, CD127 and TNF-α in response to T-cell receptor (TCR) stimulation: more preferably, further having enhanced expression levels of CD122 and/or CD215 in response to TCR stimulation.

In some preferred embodiments, the reprogrammed CD8+ T cells have enhanced proliferation in response to stimulation with IL-7 and/or IL-15.

In some preferred embodiments, the reprogrammed CD8+ T cells have an enhanced response to antigen, characterized by: (i) a higher frequency of antigen-specific CD8+ T cells that are enriched in Stem cell memory and Central memory CD8+ T cells; (ii) higher frequency of antigen-specific CD8+ T cells producing cytokines such as TNF-α and/or (iii) antigen-specific CD8+ T cells having higher survival.

In some preferred embodiments, the reprogrammed CD8+ T cells have an enhanced polyfunctionality characterized by a higher frequency of TNF-α+ and IFN-7+CD8+ T cells; preferably a higher frequency of TNF-α+, IFN-7+ and IL-2+CD8+ T cells; more preferably a higher frequency of TNF-α+, IFN-7+, IL-2+ and granzyme B+CD8+ T cells.

In some preferred embodiments, the reprogrammed CD8⁺ T cells have less mTORC1-dependency in response to TCR stimulation, characterized by a lower frequency of pS6+ cells and/or higher frequency of pS6⁻ pAKT⁺CD8+ T cells.

In some preferred embodiments, the reprogrammed CD8+ T cells have an enhanced metabolic plasticity characterized by maintenance of a higher production of TNF-α despite glucose deprivation.

In some preferred embodiments, the reprogrammed CD8+ T cells comprise a higher proportion of memory CD8+ T cells with stemness compared to the non-reprogrammed CD8+ T cells. Preferably, the proportion of memory CD8+ T cells with stemness is increased by 2-fold to up to 10-fold. Preferably, the reprogrammed memory CD8+ T cells with stemness have a higher frequency of antigen-specific CD8+ T cells and an enhanced proliferation in response to IL-7 and/or IL-15 compared to non-reprogrammed memory CD8+ T cells with sternness.

In some embodiments, the reprogrammed CD8⁺ T cells have a reduced polyfunctionality in response to TCR stimulation characterized by a lower frequency of TNF-α+ and IFN-γ+CD8+ T cells compared to non-reprogrammed CD8+ T cells.

In some embodiments, the reprogrammed CD8+ T cells have a high activation of mTORC1 and mTORC2 pathways in response to TCR stimulation, characterized by a higher frequency of pS6⁺pAKT⁺CD8+ T cells compared to non-reprogrammed CD8+ T cells.

In some embodiments, the reprogrammed CD8+ T cells have a low activation of mTORC1 and mTORC2 pathways in response to TCR stimulation, characterized by a lower frequency of pS6⁺pAKT⁺CD8+ T cells compared to non-reprogrammed CD8⁺ T cells.

In some particular embodiments, the reprogrammed CD8+ T cells have: (i) a reduced polyfunctionality in response to TCR stimulation characterized by a lower frequency of TNF-α+ and IFN-γ+CD8+ T cells and (ii) a low activation of mTORC1 and mTORC2 pathways in response to TCR stimulation, characterized by a lower frequency of pS6⁺pAKT⁺CD8+ T cells, compared to non-reprogrammed CD8+ T cells.

In some particular embodiments, the reprogrammed CD8+ T cells have: (i) an enhanced polyfunctionality in response to TCR stimulation characterized by a higher frequency of TNF-α+ and IFN-γ+CD8+ T cells and (ii) a high activation of mTORC1 and mTORC2 pathways in response to TCR stimulation, characterized by a higher frequency of pS6⁺pAKT⁺CD8+ T cells, compared to non-reprogrammed CD8⁺ T cells.

In some particular embodiments, the reprogrammed CD8+ T cells have: (i) an enhanced polyfunctionality in response to TCR stimulation characterized by a higher frequency of TNF-α+ and IFN-γ+CD8+ T cells and (ii) a low activation of mTORC1 and mTORC2 pathways in response to TCR stimulation, characterized by a lower frequency of pS6⁺pAKT⁺CD8+ T cells, compared to non-reprogrammed CD8+ T cells.

In some preferred embodiments, the reprogrammed CD8+ T cells comprise a higher frequency of effector CD8+ T cells, in particular CD127−T-bet+; LAG-3+PD-1+; HLA-DR+CD38+; Effector memory (TEM) and/or Terminal effector (TTE) CD8+ T cells, compared to the non-reprogrammed CD8+ T cells.

In some preferred embodiments, the reprogrammed CD8+ T cells comprise a lower frequency of effector CD8+ T cells, in particular CD127−T-bet+; LAG-3+PD-1+; HLA-DR+CD38+; Effector memory (TEM) and/or Terminal effector (TTE) CD8+ T cells, compared to the non-reprogrammed CD8+ T cells.

In some embodiments, the reprogrammed CD8+ T cells have enhanced antiviral or antitumoral effects. In some embodiments, the reprogrammed CD8+ T cells have enhanced efficacy for adoptive T cell therapy (ACT).

In some embodiments, the method of the invention, further comprises isolating, stimulating, expanding, engineering, and/or activating the reprogrammed CD8+ T cells. In some preferred embodiments, the method of the invention comprises stimulating the reprogrammed CD8+ T cells with IL-15.

In some embodiments, the method of the invention, further comprises, administering the reprogrammed CD8+ T cells to the subject. Preferably, wherein the reprogrammed CD8+ T cells are autologous or allogenic.

In some embodiments of the method of the invention, the subject has an infectious disease or cancer.

Another aspect of the invention relates to a pharmaceutical composition comprising an effective amount of the reprogrammed CD8+ T cells produced by the method according to the present disclosure, and a pharmaceutically acceptable vehicle and/or carrier.

Another aspect of the invention relates to a method for treatment of a patient in need thereof, comprising:
Reprogramming autologous or allogenic CD8+ T cells according to the method of the present disclosure; and
Administering the reprogrammed CD8+ T cells to the patient.

In some embodiments, the reprogrammed CD8+ T cells are expanded before administering to the subject. In some embodiments, the reprogrammed CD8+ T cells are CAR-T cells. In some embodiments, the method is for treatment of an infectious disease or cancer. In some preferred embodiments, the infectious disease is a viral disease. Preferably, the viral disease is selected from the group consisting of: HIV/AIDS, Hepatitis B, Hepatitis C, HTLV and CMV infections.

In some embodiments, the method of the invention further comprises administering an anticancer therapy, anti-infectious therapy and/or immunotherapy to the patient. In some preferred embodiments, the immunotherapy comprises therapeutic cytokines, immune checkpoint inhibitors and/or co-stimulatory antibodies. In some preferred embodiments, the anti-infectious therapy comprises antiretroviral therapy; preferably antiretroviral therapy.

Another aspect of the invention relates to a screening method for inducers of reprogramming of CD8+ T cells comprising: (a) administering a GSK3 inhibitor to a population of CD8+ T cells in vitro and (bi) measuring the level of expression of TCF-1, CCR7, CD27, CD127, and/or the level of activation of mTORC in the population of CD8+ T cells; or (b₂) measuring: (i) the level of expression of: CD127, T-bet, CD38, HLA-DR, PD-1 and/or LAG-3; (ii) the level of activation of mTORC and/or (iii) the polyfunctionality in the population of CD8+ T cell, according to the present disclosure.

### BRIEF DESCRIPTION OF THE DRAWINGS

FIG. 1. Induction of stem-like CD8⁺ T cells with high survival capacity and polyfunctionality by in vitro reprogramming. A. CD8⁺ T cells from people without HIV were incubated with medium, vehicle control, or a GSK3 inhibitor, for 12 hs and the phenotype was evaluated by flow cytometry. A representative donor from a total of 4 is shown. B. Frequencies of CD8⁺ T cell subpopulations after each treatment. C. Fold change of CD8⁺ T cell subpopulations after treatment with vehicle control or a GSK3 inhibitor, relative to medium alone condition (n=4). D. Expression of TCF-1 in CD8⁺ T cell subpopulations (n=4). E. The expression of the indicated markers in cells pre-treated with vehicle control or a GSK3 inhibitor was evaluated by flow cytometry. The fold change in the expression induced by 48 hs anti-CD3/CD28 stimulation, relative to unstimulated cells, is shown (n=5). F. Left: Flow cytometry analysis of dead cells (Aqua Live/Dead⁺) among total and T-bet+CD8⁺ T cells. Right: Fold change in dead CD8⁺ T cells induced by anti-CD3/CD28 stimulation, relative to the unstimulated condition (n=5). G. Frequencies of granzyme B⁺, TL-2⁺, IFN-γ⁺, and TNF-α⁺CD8⁺ T cells after anti-CD3/CD28 stimulation. H. Expression of 1 to 4 functions in CD8⁺ T cells after anti-CD3/CD28 stimulation (n=5). I. After reprogramming with the GSK3 inhibitor, CD8⁺ T cells were stimulated for 48 hs with anti-CD3/CD28, followed by wash and resting for 5 days. Then, cells were restimulated for another 48 hs with anti-CD3/CD28. Left: Flow cytometry analysis of proliferating (CFSE dilution) CD8⁺ T cells. Right: Frequencies of CD8⁺ T cells with 0, 1-3 or ≥4 cell divisions (n=9). J. Frequency of the total HCMV pp65-specific CD8⁺ T cell response (IFN-γ⁺ or CD107a⁺ or TNF-α⁺), after vehicle control or GSK3 inhibitor treatment, followed by HCMV pp65 peptides stimulation for 6 hs (n=6). K. Frequencies of CD8⁺ T cell subpopulations among HCMV pp65-specific CD8⁺ T cells (n=6). L. Expression of IFN-γ, CD107a, and TNF-α in HCMV pp65-specific CD8⁺ T cells (n=6). M. Frequency of the total HCMV pp65-specific CD8⁺ T cell response after HCMV pp65 peptides stimulation for 6 days (n=6). *P<0.05; **P≤0.01. NS: Not statistically significant. TSCM: Stem cell memory; TCM: Central memory; TTM: Transitional memory; TEM: Effector memory; TTE: Terminal effector.

FIG. 2. A. Flow cytometry gating strategy used to identify CD8⁺ T cell subpopulations: Naïve, Stem cell memory (TSCM), Central memory (TCM), Transitional memory (TTM), Effector memory (TEM), and Terminal effector (TTE). B. Expression of CCR7, CD27 and TCF-1 in total CD8⁺ T cells after treatment with vehicle control, or the GSK3 inhibitors BIO or TWS119, for 12 hs. The median fluorescence intensity of each marker is depicted. C. Fold change in TSCM, TCM and TEM cells induced by BIO or TWS119, relative to vehicle control (n=3 people without HIV). D. Fold change in T-bet⁺ cells among TSCM and TCM induced by anti-CD3/CD28 stimulation, relative to vehicle control, in cells previously treated with vehicle control, BIO or TWS119 (n=3). E. Fold change in T-bet⁺ cells among naïve/TSCM induced by anti-CD3/CD28 stimulation (0.2 and 1 μg/mL), relative to unstimulated cells, previous treatment with vehicle control or the GSK3 inhibitor (n=3). F. Fold change in T-bet⁺ cells among each CD8⁺ T cell subpopulation induced by anti-CD3/CD28+ICAM-1 stimulation, relative to unstimulated cells (n=5). G. Frequency of IL-2⁺TNF-α⁺ cells among each CD8⁺ T cell subpopulation after anti-CD3/CD28+ICAM-1 stimulation (n=5). H. Correlation between the frequency of PD-1⁺LAG-3⁺TIM3⁺TIGIT⁺CD8⁺ T cells and the proportion of CD8⁺ T cells with ≥4 cell divisions after restimulation (n=6). I. After reprogramming with the GSK3 inhibitor, CD8⁺ T cells were left unstimulated, stimulated once for 48 hs with anti-CD3/CD28, or stimulated twice with anti-CD3/CD28. The expression of T-bet in TCM and TTM cells is shown (n=5). *P<0.05. NS: Not statistically significant.

FIG. 3. Intrinsic effects of reprogramming in CD8⁺ T cell memory subpopulations. A. Left: Representative histograms showing the expression of CCR7, CD27, CD28, and TCF-1 in sorted TTM cells, in basal conditions. The median fluorescence intensity of each marker is depicted. Right: Summary of the expression of each marker in sorted TCM, TTM, TEM, and TTE cells after vehicle control or GSK3 inhibitor treatment, in the absence of stimulation. B. Left: Flow cytometry analysis of CD127⁺ T-bet⁻ and CD127⁻ T-bet⁺ cells after anti-CD3/CD28 stimulation. Right: Fold change in the indicated subsets among TCM, TTM, TEM, and TTE cells induced by anti-CD3/CD28 stimulation, relative to the unstimulated condition. C. Frequencies of granzyme B⁺, IFN-γ⁺, IL-2⁺, and TNF-α⁺TCM, TTM, TEM, and TTE cells after anti-CD3/CD28 stimulation. At least 5 donors were included for each comparison. *P<0.05; **P≤0.01. NS: Not statistically significant.

FIG. 4. Modulation of anabolic metabolism in reprogrammed CD8⁺ T cells. A. Principal Component Analysis (PCA) of gene expression by TCM cells treated with vehicle control or GSK3 inhibitor. B. Heat map of genes differentially expressed (P<0.05 according to a mixed effect model) in reprogrammed versus non-reprogrammed TCM cells. The relative level of each gene is shown, in unstimulated and polyclonally-stimulated cells (n=5 and 8 donors for control and reprogrammed condition, respectively). C. Histograms showing the expression of 2-NBDG, BODIPY, Mitotracker green, and Cell ROX in TCM cells after vehicle control or GSK3 inhibitor treatment, followed by resting (open histograms) or anti-CD3/CD28 stimulation (filled histograms). D. Frequencies of 2-NBDG⁺, BODIPY⁺, Mitotracker green⁺, and Cell ROX⁺CD8⁺ T cell subpopulations after stimulation (n=5). E. Left: Flow cytometry analysis of Nur77 expression in total CD8⁺ T cells stimulated with anti-CD3/CD28+ICAM-1 for 48 hs. Right: Frequency of Nur77⁺CD8⁺ T cells (n=5). F. Frequency of IL-2⁺TNF-α⁺ cells among Nur77⁻ and Nur77⁺ subsets (n=5). G. Expression of Nur77 in TCF-1⁺ and TCF-1⁻ subsets after anti-CD3/CD28+ICAM-1 stimulation (n=5). H. Flow cytometry analysis of the expression of pS6 and pAKT in total CD8⁺ T cells after vehicle control or GSK3 inhibitor treatment, followed by resting or anti-CD3/CD28 stimulation. I. Frequencies of pS6⁺pAKT⁺, pS6⁻pAKT⁺ and pS6⁻ pAKT⁻ subsets among total CD8⁺ T cells (n=7). J. Frequency of IFN-γ⁺TNF-α⁺ cells among the indicated subsets (n=7). *P<0.05; **P≤0.01. NS: Not statistically significant.

FIG. 5. Heat map of genes differentially expressed in reprogrammed versus non-reprogrammed TTM (A), TEM (B), and TTE (C) cells. The relative level of each gene is shown, in unstimulated and polyclonally-stimulated cells. At least 6 people without HIV were included for each comparison.

FIG. 6. Left: Flow cytometry analysis of the expression of IL-2 and IFN-7 among TNF⁺CD8⁺ T cells, after anti-CD3/CD28 stimulation. Middle: Histograms showing the expression of pS6 in the indicated subsets, in reprogrammed and non-reprogrammed cells. Right: Frequency of pS6+ cells among the indicated subsets (n=6 people without HIV). *P<0.05. NS: Not statistically significant.

FIG. 7. Enhanced functionality and survival of reprogrammed HIV-specific CD8⁺ T cells. A. Left: Expression of CCR7, CD27 and TCF-1 in non-reprogrammed (gray histograms) and reprogrammed (red histograms) HIV dextramer⁺CD8⁺ T cells. Right: UMAP plots generated from HIV dextramer⁺CD8⁺ T cells after data concatenation (n=4). Vehicle control and GSK3 inhibitor treatments, as well as CD8⁺ T cell subpopulations were identified by manual gating and projected into the UMAP space. B. Summary of the expression of CCR7, CD27 and TCF-1 in HIV dextramer⁺CD8⁺ T cells (n=7). C. Frequencies of CD8⁺ T cell subpopulations among HIV dextramer⁺CD8⁺ T cells (n=7). D. Left: Flow cytometry analysis of the expression of IFN-7 and CD107a by CD8⁺ T cells from a non-controller individual, after vehicle control or GSK3 inhibitor treatment, followed by Gag peptides stimulation for 6 hs. Right: Frequency of the total HIV Gag-specific CD8⁺ T cell response (IFN-γ⁺ or CD107a⁺ or IL-2⁺ or TNF-α⁺) (n=11). E. UMAP plots generated from HIV Gag-specific CD8⁺ T cells (IFN-γ⁺ and/or CD107a⁺) after data concatenation (n=3). F. Frequencies of CD8⁺ T cell subpopulations among HIV Gag-specific CD8⁺ T cells (n=5). G. Left: Flow cytometry analysis of the expression of IFN-γ and TNF-α by CD8⁺ T cells from a non-controller individual, after vehicle control or GSK3 inhibitor treatment, followed by Gag peptides stimulation for 6 hs. Right: Frequency of TNF-α⁺ HIV Gag-specific CD8⁺ T cells (n=12). H. Expression of CD107a, IFN-γ, granzyme B, IL-2, and TNF-α in HIV Gag-specific CD8⁺ T cells (at least 5 donors in each comparison). I. Frequency of polyfunctional CD8⁺ T cells after Gag peptides stimulation for 6 hs (n=6). J. Left: Flow cytometry analysis of the viability of proliferating HIV Gag-specific CD8⁺ T cells after Gag peptides stimulation for 6 days. Right: Frequency of dead HIV Gag-specific CD8⁺ T cells (n=6). K. Frequency of the total HIV Gag-specific CD8⁺ T cell response (live IFN-γ⁺ or IL-2⁺ or TNF-α⁺) after Gag peptides stimulation for 6 days (n=8). *P<0.05; ***P≤0.001. NS: Not statistically significant.

FIG. 8. Reprogrammed HIV-specific CD8⁺ T cells depend less on mTORC1 and glucose metabolism. A. Left: Flow cytometry analysis of the expression of pS6 and pAKT in HIV Gag-specific CD8⁺ T cells (IFN-γ+ and/or IL-2+) after vehicle control or GSK3 inhibitor treatment, followed by Gag peptides stimulation for 6 hs. Right: Frequencies of the indicated subsets among HIV Gag-specific CD8⁺ T cells (from n=6 people with HIV-1). B. Expression of pS6 and pAKT in HIV Gag-specific CD8⁺ T cells. C. Left: Flow cytometry analysis of the expression of CD107a and IFN-γ after vehicle control or GSK3 inhibitor treatment, followed by Gag peptides stimulation for 6 hs in the presence or absence of glucose. Right: Frequency of the total HIV Gag-specific CD8⁺ T cell response (IFN-γ⁺ or CD107a⁺ or IL-2⁺ or TNF-α⁺) (n=6). D. Ratio of the frequency of the total HIV Gag-specific CD8⁺ T cell response between no glucose versus glucose conditions. E. Frequency of TNF-α⁺ HIV Gag-specific CD8⁺ T cells after Gag peptides stimulation for 6 hs in the absence of glucose (n=6). *P<0.05. NS: Not statistically significant.

FIG. 9. Expression of CD122 (A) and CD215 (B) in each CD8⁺ T cell subpopulation (n=5 people without HIV). C. Fold change in the expression of T-bet in response to IL-15 (relative to unstimulated cells) in HIV dextramer⁺CD8⁺ T cells from non-controllers (n=4). *P<0.05; NS: Not statistically significant.

FIG. 10. Superior response to γ-chain cytokines by reprogrammed HIV-specific CD8⁺ T cells. A. Top: Flow cytometry analysis of the expression of Eomes and CD122 in total CD8⁺ T cells after vehicle control or GSK3 inhibitor treatment. Bottom: Frequency of Eomes⁺CD122⁺ cells among each CD8⁺ T cell subpopulation (n=5 people without HIV). B. Top: Flow cytometry analysis of the proliferation of CD8⁺ T cells from a person without HIV after vehicle control or GSK3 inhibitor treatment, followed by stimulation with IL-7 or IL-15 for 6 days. Bottom: Division index of CD8⁺ T cell subpopulations in response to IL-7 or IL-15 (n=5). C. Flow cytometry analysis of the proliferation of HIV dextramer⁺CD8⁺ T cells from a HIV-1 non-controller individual, after vehicle control or GSK3 inhibitor treatment, followed by stimulation with IL-15 for 6 days. D. Frequencies of proliferating HIV dextramer⁺CD8⁺ T cells in response to IL-15, in cells derived from non-controllers (n=4). *P<0.05. NS: Not statistically significant.

FIG. 11. Superior response to PD-1 blockade by reprogrammed HIV-specific CD8⁺ T cells. A. Frequency of proliferating HIV Gag-specific CD8⁺ T cells after vehicle control or GSK3 inhibitor treatment, followed by Gag peptides stimulation for 6 days in the presence or absence of an anti-PD1 blocking antibody. B. TNF-α expression in HIV Gag-specific CD8⁺ T cells under the conditions indicated. *P<0.05. NS: Not statistically significant.

FIG. 12. Screening molecules for reprogramming human CD8⁺ T cells Total CD8⁺ T cells were purified and treated for 12 hs with titrated doses of GSK3 inhibitors or DMSO vehicle control. After drug washout, cells were stimulated for 48 hs with plate-bound anti-CD3/anti-CD28 antibodies (at 1 mg/mL), followed by flow cytometry analyses. A. Upon stimulation, intracellular cytokine and phospho-protein staining was performed, the latter for the detection of phospho-S6 and phospho-AKT proteins (indicative of activation of mTORC1 and mTORC2 pathways, respectively). The fold change in the frequency of IFN-γ⁺TNF-α⁺ and pS6⁺pAKT⁺ cells induced by the treatment with each GSK3 inhibitor, relative to DMSO control, was analyzed. The dot plot shows the correlation between the fold change in the frequency of IFN-γ⁺TNF-α⁺ and pS6⁺pAKT⁺ cells. Each dot represents the median levels obtained with the respective GSK3 inhibitor (at least 3 donors per condition). B. Fold change in the expression of the indicated markers and memory phenotypes induced by anti-CD3/anti-CD28 antibody stimulation relative to unstimulated cells (at least 3 donors per condition). TCM: T central memory; TEM: T effector memory; TTM: T transitional memory; TTE: T terminal effector; TSCM: T stem cell memory.

### DETAILED DESCRIPTION OF THE INVENTION

The invention encompasses a method for reprogramming CD8+ T cells to enhance their therapeutic potential, as well as reprogrammed CD8+ T cells with enhanced therapeutic potential produced by the method and their various uses in particular in methods of adoptive T cell therapy of cancer and infectious diseases.

As used herein, “CD8+ T cells reprogramming” refers to the induction of CD8+ T cells having enhanced sternness and functional properties or a modulation (enhancement or repression) of effector cells.

T cell “stemness” refers the capacity of a T cell to self-renew, to differentiate into multiple downstream cell types, and to interact with its environment to maintain a balance between quiescence, proliferation, and regeneration.

By “CD8+ T cells having enhanced stemness and functional properties” or “CD8+ T cells with enhanced potential” it is meant herein CD8+ T cells with enhanced capacity to react to antigen stimulation, eliminate pathogenic or tumoral cells and persist, in particular in pathological conditions such as pathogenic or tumoral conditions.

In some embodiments, CD8+ T cells reprogramming encompasses the enrichment of CD8+ T cells in memory CD8+ T cells with stemness, wherein the memory CD8+ T cells have enhanced stemness and functional properties. The memory CD8+ T cells with stemness or stem-like memory CD8+ T cells are in particular Stem cell memory (TSCM) and Central memory (TCM).

By “modulation of effector-like phenotypes in CD8+ T cells”, “modulation of effector cells” or “modulation of effector CD8+ T cells”, it is meant the enhancement or repression of effector CD8+ T cells. The modulation may involve an increase or decrease of effector CD8+ T cell number or activity. The effector CD8+ T cells are in particular Terminal effector (TTE) and Effector memory (TEM).

By “reprogrammed CD8+ T cells” it is meant herein the CD8+ T cells having enhanced potential produced by the method of the invention. The reprogrammed CD8+ T cells produced by the method of the invention have modified transcriptional, translational and metabolic signatures compared to non-reprogrammed CD8+ T cells, as disclosed in the examples.

CD8+ T cell with stemness and optimal functional properties are key for long term control of diseases such as infected diseases and or cancer. However, antigen-specific CD8+ T cells with a skewed or dysfunctional profile are found in tumors or chronic viral infections such as HIV-1 non-controllers. Controller patients such as HIV-1 controller are characterized by virus-specific CD8+ T cells with enhanced stemness and functional properties compared to non-controllers. As shown in the examples, reprogrammed CD8+ T cells with enhanced stemness and functional properties can be induced in HIV-1 non-controller individuals using the reprogramming method of the invention. Therefore, CD8+ T cell programming according to the method of the invention is useful to potentiate the therapeutic efficacy of CD8+ T cells in adoptive T cell therapy, in particular to improve the antimicrobial, in particular antiviral effect or antitumoral effects of CD8+ T cells in pathological conditions such as infectious diseases and cancer.

CD8+ T cells comprise distinct types, naïve, effector and memory. Naïve CD8+ T cells are continuously circulating throughout the body migrating through the blood and secondary lymphoid tissues. Once a naïve T cell encounters its cognate antigen on an antigen presenting cell within a secondary lymphoid organ, the CD8+ T cell undergoes a number of molecular changes leading to T cell being in an activated state and differentiating into a number of different subpopulations. Memory CD8+ T cells can be defined as long-lived CD8+ T cells that are antigen-specific and provide an enhanced protective response when the same antigen is encountered again. These cells persist in the absence of antigen but maintain a distinct phenotype and elevated precursor frequency, which is one way of distinguishing them from the naïve CD8+ T cell population (Review in Samji et al., Immunol. Lett., 2017, 185, 32-39).

The different types of CD8+ T cells may be distinguished by their phenotype based on expression of specific surface markers. T cells may be defined as CD3+ cells. Human naïve CD8+ T cells may be defined as CD45RA+, CCR7+, CD27+ and CD95−. Human effector CD8+ T cells or Terminal effector (TTE) may be defined as CD45RA+, CCR7−, CD27−. Human memory CD8+ T cells are divided in four subsets from the less differentiated to the more differentiated memory cells: Stem cell memory (TSCM), Central memory (TCM), Transitional memory (TTM), and Effector memory (TEM). Stem cell memory (TSCM) CD8+ T cells may be defined as CD45RA+, CCR7+, CD27+ and CD95+. Central memory (TCM) CD8+ T cells may be defined as CD45RA−, CCR7+, CD27+. Transitional memory (TTM) CD8+ T cells may be defined as CD45RA−, CCR7−, CD27+. Effector memory (TEM) CD8+ T cells may be defined as CD45RA−, CCR7−, CD27−. Human Stem cell memory (TSCM) and Central memory (TCM) CD8+ T cells which are the less differentiated memory CD8+ T cells are characterized by their sternness; they are also the only CD8+ T cells with a CCR7+ and CD27+ phenotype (Galetti et al., Nat Immunol, 2020, 21, 1552-1562).

The different types and subtypes of CD8+ T cells as disclosed herein can be analysed and sorted by routine techniques known in the art such as flow cytometry assisted cell sorting or magnetic cell separation using appropriate antibodies as disclosed in the examples, description of the drawings and hereafter. Flow cytometry such as flow cytometry assisted cell sorting is used to determine the proportion (percentage, frequency) of the different CD8+T subsets (naïve, TSCM, TCM, TTM, TEM, TTE) in the reprogrammed versus non-reprogrammed CD8⁺ T cells using the specific cell-surface markers as disclosed herein. Flow cytometry is also used to determine the expression levels of cell-surface and intracellular markers in the reprogrammed versus non-reprogrammed CD8⁺ T cells as disclosed herein. Flow cytometry is also used to measure mTORC1 and mTORC2 activation levels. For example, flow cytometry analysis, can be performed according to the following protocol as disclosed in the examples. After cultures, cells were stained with the LIVE/DEAD Fixable Aqua Dead Cell Stain kit (Thermo Fisher Scientific), with anti-CD3 Alexa Fluor 700 or APC efluor 780 and anti-CD8 PE Texas Red antibodies, accordingly. For phenotype analyses, cells were additionally stained with anti-CCR7 PE Cy7, anti-CD45RA APC H7, anti-CD27 PerCP Cy5.5, anti-CD95 APC, or anti-CD127 Alexa Fluor 488 antibodies, incubated for 15 minutes at room temperature. Additional surface staining panels included anti-HLA-DR Superbright 780, anti-CD38 Superbright 600, anti-PD-1 BV421, anti-LAG-3 APC efluor 780, anti-TIM3 PE Cy7, and anti-TIGIT BV786 antibodies, or anti-CD122 PE and anti-CD215 FITC antibodies. The BD Transcription Factor Buffer Set kit (BD Biosciences) was used for cell fixation and permeabilization. Intracellular staining panels for detection of transcription factors included anti-TCF-1 PE, anti-T-bet V450, anti-Eomes APC, anti-TOX efluor 660, anti-BLIMP1 CF594, anti-BCL6 Alexa Fluor 488, or anti-Nur77 Alexa Fluor 647, accordingly. Intracellular cytokine staining panels included anti-IFN-γ PE Cy7 or V450, anti-IL-2 APC-R700, anti-TNF-αPerCP Cy5.5, or anti-granzyme B Alexa Fluor 647 antibodies, accordingly. For mTORC1/mTORC2 pathway analysis, cells were fixed and permeabilized with Phosflow fix/perm buffers (BD Biosciences) and cells were stained with anti-pS6 Ser235/236 Pacific blue and anti-pAKT Ser473 FITC antibodies (Cell Signaling). Cell acquisition was performed using LSR II or ARIA III flow cytometers (both from BD Biosciences), and data were analyzed with FlowJo v.10 software (BD Biosciences).

As used herein, a change in the level of a factor refers to a significant change, preferably of at least 1.1-fold (1.2, 1.3, 1.4, 1.5 or more) compared to control.

One aspect of the invention relates to a method for reprogramming CD8+ T cells, comprising stimulating a Wnt/TCF-1 and/or mTORC2 signaling pathways, and/or inhibiting a mTORC1 signaling pathway in a population of CD8+ T cells.

Reprogrammed CD8+ T cells having enhanced stemness and functional properties are induced following stimulation of the Wnt/TCF-1 and/or mTORC2 signaling pathways, and/or inhibition of the mTORC1 signaling pathway in the population of CD8+ T cells as disclosed in the examples. The method of the invention does not require any other stimulation such as TCR stimulation (e.g., activation) to induce the reprogramming of the CD8+ T cells. The enhanced stemness of the reprogrammed CD8+ T cells is stable even after activation. For example, a glycogen synthase kinase-3 (GSK3) inhibitor can be used to stimulate the Wnt/TCF-1 and mTORC2 signaling pathways and inhibit the mTORC1 signaling pathway. GSK3 inhibition may be achieved by transient exposure to GSK3 inhibitor. Inhibition of GSK3 promotes memory CD8+ T cells with stemness and enhances the functional properties of CD8+ T cells as shown in the examples.

Reprogrammed CD8+ T cells having a modulation of effector CD8+ T cells may also be induced following stimulation or inhibition of mTORC1 and mTORC2 signaling pathways, in the population of CD8+ T cells as disclosed in the examples. The method of the invention does not require any other stimulation such as TCR stimulation (e.g., activation) to induce the reprogramming of the CD8+ T cells. For example, a glycogen synthase kinase-3 (GSK3) inhibitor can be used to stimulate or inhibit mTORC1 and mTORC2 signaling pathways. GSK3 inhibition may be achieved by transient exposure to GSK3 inhibitor. Inhibition of GSK3 modulates (promotes or represses) effector CD8⁺ T cells as shown in the examples.

In various embodiments, the population of CD8+ T cells is from a subject, preferably a human. In some particular embodiments, the subject is a patient as disclosed herein.

The CD8+ T cells, preferably human CD8+ T cells, can be tissue resident or circulating CD8+ T cells. In some embodiments, the CD8+ T cells are circulating CD8+ T cells, in particular peripheral blood CD8+ T cells. In some embodiments, the CD8+ T cells are engineered T cells in particular CAR-T cells as disclosed herein.

In some preferred embodiments, the reprogramming method of the invention comprises: administering a glycogen synthase kinase-3 (GSK3) inhibitor to a population of CD8+ T cells.

In some preferred embodiments, the step of stimulating a Wnt/TCF-1 and/or mTORC2 signaling pathways, and/or inhibiting a mTORC1 signaling pathway, in particular using a GSK3 inhibitor is performed in vitro. In some embodiments, the population of CD8+ T cells is obtained from a subject sample. In various embodiments, the sample is a body fluid or tissue sample including tumor sample. In some embodiments, the sample is a blood sample. In various embodiments, the population of CD8+ T cells is isolated or purified. CD8+ T cells can be isolated from tissue samples and cell samples by routine techniques in the art. For example, CD8+ T cells can be extracted from lymphoid tissues or tumors and isolated using negative or positive immunomagnetic selection. For example, CD8+ T cells can be isolated from human peripheral blood mononuclear cells (PBMCs) using negative or positive immunomagnetic selection as disclosed in the examples.

By “GSK3 inhibitor” it is meant herein any agent able to decrease specifically GSK3 expression and/or biological activity, in particular that results in increased expression of TCF-1, CD127 (IL-7Rα), activation of mTORC2, and/or inhibition of mTORC1 in CD8+ T cells. GSK3 inhibitor can also be identified by measuring the expression level of beta-catenin (downstream target of GSK3) in cells. In some embodiments, GSK3 inhibitor may induce inhibition of mTORC2 and/or activation of mTORC1; in particular embodiments GSK3 inhibitor may induce activation of mTORC2 and mTORC1 pathways; in other particular embodiments, GSK3 inhibitor may induce inhibition of mTORC2 and mTORC1 pathways.

The GSK3 inhibitor may target the GSK3a or GSK30 isoforms or both. Inhibitors of GSK3 include chemicals such as small organic molecules; small RNA molecules such as siRNA, miRNA, ribozymes, modified or unmodified; epigenome editing enzyme complexes such as derived from CRISPR/Cas, TALENs, or Zinc-Finger nucleases; peptides, antibodies, aptamers or other antagonists.

GSK3 inhibitor suitable for CD8+ T cell reprogramming to induce CD8+ T cells having enhanced stemness and functional properties can be determined by measuring the level of expression of TCF-1, CCR7, CD27 and/or CD127, or activation of mTORC (mTORC1, mTORC2) in CD8+ T cells treated or not with said GSK3 inhibitor. These factors are measured following TCR stimulation, except for TCF-1, CCR7 and CD27 that may be measured with or without TCR stimulation. CD8+ T cell reprogramming with said GSK3 inhibitor is effective when TCF-1, CCR7, CD27, CD127, and/or mTORC2 are up-regulated, and/or mTORC1 is down-regulated. For example, the level of expression of TCF-1, CCR7, CD27, and/or CD127 or activation of mTORC is at least 1.1-fold (1.2, 1.3, 1.4, 1.5 or more) higher (TCF-1, CCR7, CD27, CD127 or mTORC2) or lower (mTORC1) in CD8+ T cells treated with the GSK3 inhibitor than in non-treated cells (control). The level of expression of the CCR7, CD27, CD127, and TCF-1 mRNA or protein and the level of activation of mTORC1 and mTORC2 may be determined according to the routine techniques, well-known of the person skilled in the art. mRNA expression level may be measured by nucleic acid amplification methods such as RT-PCR, in particular real time Q-PCR, RT-qPCR. Protein expression level may be measured by quantitative or semi-quantitative immunoassays such as flow cytometry, as disclosed in the examples. mTORC pathway activation can be measured by analyzing the phosphorylation of ribosomal S6 (pS6 Ser235/236) and AKT (pAKT Ser473) proteins, markers of activation of mTORC1 and mTORC2, respectively, by flow cytometry, as disclosed in the examples. GSK3 inhibitor suitable for CD8+ T cell reprogramming may also be determined by treating CD8+ T cells with the GSK3 inhibitor followed by TCR stimulation, and measuring the survival, polyfunctionality, proliferation capacity, metabolic plasticity, response to antigen, response to γ-chain cytokines, response to immune check-point modulators, cytotoxic effect, and/or mTORC1-dependency of the CD8+ T cells. These properties may be measured according to the routine techniques, well-known of the person skilled in the art as disclosed herein. CD8+ T cell reprogramming with said GSK3 inhibitor is effective when the survival, polyfunctionality, proliferation capacity, metabolic plasticity, response to antigen, response to γ-chain cytokines, response to immune check-point modulators, and/or cytotoxic effect is increased in the CD8+ T cells by the GSK3 inhibitor, and/or when mTORC1-dependency is decreased in the CD8+ T cells by the GSK3 inhibitor. Therefore, a person skilled in the art can easily determine GSK3 inhibitors suitable for CD8+ T cell reprogramming.

GSK3 inhibitor suitable for CD8+ T cell reprogramming to modulate (enhance or repress) effector CD8+ T cells can be determined by measuring: (i) the level of expression of: CD127; the effector transcription factor T-bet; activation marker(s), in particular CD38 and HLA such as HLA-DR; and/or inhibitory receptor(s), in particular PD-1 and LAG-3; (ii) level of activation of mTORC (mTORC1, mTORC2) and/or (iii) polyfunctionality in CD8+ T cells treated or not with said GSK3 inhibitor. These factors are measured following TCR or cytokine stimulation. For example, effector phenotype and/or properties are enhanced with said GSK3 inhibitor when the frequency of CD127−T-bet+; LAG-3+PD-1+; HLA-DR+CD38+; Effector memory (TEM) and/or Terminal effector (TTE) cells is higher in CD8+ T cells treated with the GSK3 inhibitor than in non-treated cells (control). For example, effector phenotype and/or properties are enhanced with said GSK3 inhibitor when the reprogrammed CD8+ T cells have: (i) an enhanced polyfunctionality in response to TCR or cytokine stimulation characterized by a higher frequency of TNF-α+ and IFN-γ+CD8+ T cells and (ii) a high concomitant activation of mTORC1 and mTORC2 pathways in response to TCR or cytokine stimulation, characterized by a higher frequency of pS6⁺pAKT⁺CD8+ T cells, compared to non-reprogrammed CD8+ T cells. For example, effector phenotype and/or properties are repressed with said GSK3 inhibitor when the frequency of CD127−T-bet+; LAG-3+PD-1+; HLA-DR+CD38+; Effector memory (TEM) and/or Terminal effector (TTE) cells is lower in CD8+ T cells treated with the GSK3 inhibitor than in non-treated cells (control). For example, effector phenotype and/or properties are repressed with said GSK3 inhibitor when the reprogrammed CD8+ T cells have: (i) a reduced polyfunctionality in response to TCR or cytokine stimulation characterized by a lower frequency of TNF-α+ and IFN-γ+CD8+ T cells and (ii) a low activation of mTORC1 and mTORC2 pathways in response to TCR or cytokine stimulation, characterized by a lower frequency of pS6⁺pAKT⁺CD8+ T cells, compared to non-reprogrammed CD8+ T cells. These properties may be measured according to the routine techniques, well-known of the person skilled in the art as disclosed herein.

GSK3 inhibitors have diverse chemotypes and mechanisms of action and include for example compounds isolated from natural sources, cations, synthetic small-molecule, ATP-competitive inhibitors, non-ATP-competitive inhibitors, and substrate-competitive inhibitors (Review in Front. Mol. Neurosci., 31 Oct. 2011, doi:10.3389):
Small metal cation inhibitors include: Lithium (IC50 1-2 mM), the drug of reference for bipolar disorders; Zinc (IC50 15 μM); tungstate;
ATP-competitive inhibitors derived from marine organism include: Indirubins (IC50 5-50 nM) and synthetic analogs thereof, in particular 6-bromoindirubin-3′-oxime or BIO (IC50 1.5 μM) and 6-bromoindirubin-3′-acetoxime (BIO-acetoxime or BIA); Hymenialdisine (Pyrroloazepine; IC50 10 nM) and Debromohymenialdisine; Dibromocantharelline (IC50 3 μM); Meridianins such as Meridianine A;
ATP-competitive inhibitors derived from organic synthesis include: Pyridine and Pyrimidine compounds including Pyrrole-aminopyrimidines such as the aminopyrimidines CT98014 (CHIR98014), CT98023 (CHIR98023), CT99021 (CHIR-99021), the Pyrrolopyrimidine TWS119 (IC50 0.6-7 nM); the phenylaminopyrimidine CGP 60474 (CAS No. 164658-13-3); Maleimide derivatives including Arylindolemaleimides (Anilinomaleimides, bisarylmaleimides) such as SB-216763, SB-415286 (IC50 34-77 nM)); Bisindolylmaleimide such as GF 109203X (CAS No. 133052-90-1) and Staurosporine (CAS No. 62996-74-1); 9-Ing-41 (elraglusib) and LY-2090314; Thiazoles, in particular AR-A014418 (IC50 104 nM), AZD-1080; Paullones such as Kenpaullone, Alsterpaullone (Benzaepinone), Cazpaullone (IC50 4-80 nM); Aloisines such as Aloisine A (Pyrrolopyrazine; IC50 0.5-1.5 μM); Pyrazine such as AZD2858 (CAS No. 486424-20-8); Oxindoles (indolinone) such as SU9516 (CAS No. 666837-93-0); Oxazole-carboxamide derivatives such as PF-04802367 (CAS No. 1962178-27-3);
Non-ATP-competitive inhibitors include Manzamines such as Manzamine A (IC50 1.5 μM) and Furanosesquiterpenes such as Palinurin and Tricantin (IC50 4.5-7.5 μM), derived from marine organism; Thiadiazolidinones such as TDZD-8, NP00111, NP031115, NP031112 (tideglusib) (IC50 2-4 μM); Halomethylketones such as HMK-32 (IC50 1.5 μM), from organic synthesis;
Substrate competitive inhibitors include peptides such as L803-mts (IC50 40 μM); and pharmaceutical acceptable salts thereof;
other inhibitors such as Flavones, in particular Flavopiridol hydrochloride hydrate (CAS No. 131740-09-5).

Some GSK3 inhibitors are used in human therapy and others currently tested in clinical trials. The method of the invention may use any one of the various GSK3 inhibitors known in the art or a combination thereof.

In some embodiments, the GSK3 inhibitor is selected from the group consisting of: Maleimide derivatives, in particular Anilinomaleimides and Bisindolylmaleimides; ions (competition with Mg2+); Pyrroloazepines; Flavones; Benzazepinones; Bis-Indoles, in particular Indirubins; Pyrrolopyrazines; Pyridine and Pyrimidine derivatives, in particular Aminopyrimidines, Pyrrolopyrimidines and Phenylaminopyrimidines; Oxindoles (indolinone); Thiazoles; and further Pyrazines, Thiadiazolidinones and Oxazole-carboxamides.

In some particular embodiments, the GSK3 inhibitor is selected from the group consisting of: SB-415286 (CAS No. 264218-23-7), Lithium carbonate, 10Z-Hymenialdisine (CAS No. 82005-12-7), Flavopiridol hydrochloride hydrate (CAS No. 131740-09-5), Alsterpaullone (CAS No. 237430-03-4), BIO and BIO-Acetoxime (CAS No. 667463-85-6), Aloisine A (CAS No. 496864-16-5), CHIR 98014 (CAS No. 252935-94-7), SU9516 (CAS No. 377090-84-1), AR-A014418 (CAS No. 487021-52-3), Staurosporine (CAS No. 62996-74-1), GF 109203X (CAS No. 133052-90-1), CGP 60474 (CAS No. 164658-13-3), TWS119, functional analogs or derivatives thereof, and combinations thereof; and further AZD2858 (CAS No. 486424-20-8), NP031112 (tideglusib; CAS No. 865854-05-3), SB-216763, PF-04802367, functional analogs or derivatives thereof, and combinations thereof.

In some embodiments, the GSK3 inhibitor is a small-molecule ATP-competitive inhibitor or a combination thereof, preferably chosen from indirubins, aminopyrimidines, and combinations thereof.

In some embodiments, the GSK3 inhibitor is an indirubin of formula (I) or a pharmaceutically acceptable salt thereof:

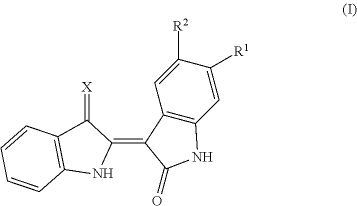

wherein
R¹ is a halogen, or a vinyl group (—CH═CH₂);
R² is selected from the group consisting of H, halogen, amino, nitro and C₁₋₅ alkyl; and
X is O or N—OR³, wherein R³ is selected from the group consisting of H, -(A)-R⁴, —C(O)R⁵ and —C(O)N(R⁶, R⁷); with
A being a non-substituted C₁₋₅ alkylene group or a C₁₋₅ alkylene group substituted by one or several A¹ groups, A¹ being selected from the group consisting of halogen, OH, OR⁸ and NH₂; R⁸ being a C₁₋₅ alkyl;
R⁴ being selected from the group consisting of H, halogen, OH and N(R⁶, R⁷);
R⁵ being a C₁₋₅ alkyl; and
R⁶ and R⁷ being an identical or a different non-substituted C₁₋₅ alkyl group or a C₁₋₅ alkyl group substituted by one or more A¹ groups as defined above, or wherein R⁶ and R⁷ are part of a 5-6 cycle optionally comprising another heteroatom selected from O or N.

The pharmaceutically acceptable salts comprise, among others, chlorides, acetates, succinates, citrates of the above disclosed indirubins.

In some embodiments, R¹ is Br. In other embodiments R¹ is halogen, preferably Br, and X is N—OR³, being R³ as defined above herein. Preferably the indirubin of formula (I) is 6-bromo-indirubin-3′-oxime (BIO) or 6-bromo-indirubin-3′-acetoxime (BIO-acetoxime).

In some embodiments R¹ is halogen or a vinyl group (—CH═CH₂), R² is H, and X is defined as above herein. Preferably R¹ is Br, R² is H, and X is defined as above herein.

In other embodiments R¹ is halogen or a vinyl group (—CH═CH₂), R² is halogen, nitro or C₁₋₅ alkyl; and X is defined as above herein.

In some embodiments, X is O. Preferably, for said embodiments, when X is O:
R¹ is Cl, Br, I or vinyl (—CH═CH₂) and R² is H; or
R¹ is Cl, Br or I and R² is selected from the group consisting of halogen, amino, nitro and C₁₋₅ alkyl.

In some embodiments, X is N—OR³ and R³ is H. In said embodiments, when X is N—OR³ and R³ is H, R¹ is Cl, Br, I or vinyl (—CH═CH₂), and R² is selected from the group consisting of H, halogen, amino, nitro and Ci₅ alkyl. Preferably, when X is N—OR³ and R³ is H, R¹ is Br and R² is selected from the group consisting of H, halogen, amino, nitro and C₁₋₅ alkyl. More preferably, X is N—OR³ with R³ being H, R¹ is Br and R² is H.

In other embodiments, X is N—OR³ and R³ is —C(O)R⁵ and R⁵ is C₁₋₅ alkyl. In said embodiments, when X is N—OR³ and R³ is —C(O)R⁵, with R⁵ being C₁₋₅ alkyl, R¹ is Cl, Br or I and R² is selected from the group consisting of H, halogen, amino, nitro and C₁₋₅ alkyl. Preferably, when X is N—OR³ and R³ is —C(O)R⁵, with R⁵ being C₁₋₅ alkyl, R¹ is Br and R² is selected from the group consisting of H, halogen, amino, nitro and C₁₋₅ alkyl. More preferably, X is N—OR³ with R³ being —C(O)R⁵ and R⁵ being methyl, R¹ is Br and R² is H.

In some embodiments R³ is -(A)-R⁴, wherein A and R⁴ are defined as above herein. Preferably, A is a non-substituted C₁₋₅ alkylene group or a C₁₋₅ alkylene group substituted with one or more OH. In some preferred embodiments, R⁴ is halogen, preferably Br, or OH. In other preferred embodiments, R⁴ is N(R⁶R⁷), wherein R⁶ and R⁷ are identical or different non-substituted C₁₋₅ alkyl or a C₁₋₅ alkyl substituted by one or more A¹ groups as defined herein.

In other embodiments R³ is —C(O)N(R⁶, R⁷) wherein R⁶ and R⁷ are identical or different non-substituted C₁₋₅ alkyl or a C₁₋₅ alkyl substituted by one or more A¹ groups as defined herein.

In some particular embodiments, R⁶ and R⁷ are part of a cycle and form a pyrrole, a morpholinyl, or a piperazinyl radical, said radical being optionally substituted by one or several A¹, as defined herein, and the piperazinyl radical being optionally substituted on the nitrogen by a C₁₋₅ alkyl group, which can in turn be substituted by one or several Ai groups as previously defined herein.

In a particular embodiment the indirubin of formula (I) is selected from the group consisting of 6-bromo-indirubin, 6-bromo-indirubin-3′-oxime (BIO), 6-bromo-indirubin-3′methoxime, 6-bromo-indirubin-3′-acetoxime (BIO-acetoxime), 6-chloro-indirubin, 6-chloro-indirubin-3′-oxime, 6-chloro-indirubin-3′-acetoxime, 6-iodo-indirubin, 6-iodo-indirubin-3′-oxime, 6-iodo-indirubin-3′-acetoxime, 6-vinylindirubin-3′-oxime, 6-vinylindirubin-3′-acetoxime, 6-fluoroindirubin-3′-oxime, 6-fluoroindirubin-3′-acetoxime, 6-bromo-5-methylindirubin, 6-bromo-5-methylindirubin-3′-oxime, 6-bromo-5-methylindirubin-3′-acetoxime, 5,6-dichloroindirubin, 5,6-dichloroindirubin-3′-oxime, 5,6-dichloroindirubin-3′-acetoxime, 6-bromo-5-nitroindirubin, 6-bromo-5-nitroindirubin-3′-oxime, 6-bromo-5-nitroindirubin-3′-acetoxime, 6-bromo-5-aminoindirubin, 6-bromo-5-aminoindirubin-3′-oxime, 5,6-dibromoindirubin, 6-bromoindirubin-3′-[O-(2-bromoethyl)-oxime], 6-bromoindirubin-3′-[O-(2-hydroxyethyl)-oxime], 6-bromoindirubin-3′-[O-(2,3-dihydroxypropyl)-oxime], 6-bromoindirubin-3′-[0-(N,N-diethylcarbamyl)-oxime], 6-bromoindirubin-3′-[O-(2-dimethylaminoethyl)-oxime], 6-bromoindirubin-3′-[O-(2-diethylaminoethyl)-oxime], 6-bromoindirubin-3′-[O-(2-pyrrolidin-1-ylethyl)oxime], 6-bromoindirubin-3′-[O-(2-morpholin-1-ylethyl)oxime], 6-bromoindirubin-3′-[O-(2-(N,N-(2-hydroxyethyl)aminoethyl)oxime], 6-bromoindirubin-3′-(O-{2-[N-methyl, N-(2,3-dihydroxypropyl)amino]ethyl}oxime], 6-bromoindirubin-3′-[O-(2-piperazine-1-ylethyl)oxime], 6-bromoindirubin-3′-(0-[2-(4-methyl-piperazin-1-yl)ethyl]oxime), 6-bromoindirubin-3′-(O-{2-[4-(2-hydroxyethyl)piperazin-1-yl]ethylIoxime), 6-bromoindirubin-3′-(O-{2-[4-(2-methoxyethyl)piperazin-1-yl]ethyl}oxime), 6-bromoindirubin-3′-[O-(2-(4-[2-(2-hydroxyethoxy)-ethyl]piperazin-1-yl)ethyl)oxime], 6-bromoindirubin-3′-[O-(2-dimethylaminoethyl)oxime]hydrochloride, 6-bromoindirubin-3′-[0-(2-diethylaminoethyl)oxime]hydrochloride, 6-bromoindirubin-3′-[O-(2-pyrrolidin-1-ylethyl)oxime]hydrochloride, 6-bromoindirubin-3′-[O-(2-morpholin-1-ylethyl)oxime]hydrochloride, 6-bromoindirubin-3′-[O-(2-(N,N-(2-hydroxyethyl)aminoethyl)oxime], 6-bromoindirubin-3′-(O-{2-[N-methyl, N-(2,3-dihydroxypropyl)amino]ethyl}oxime]hydrochloride, 6-bromoindirubin-3′-[O-(2-piperazine-1-ylethyl)oxime]dihydrochloride, 6-bromoindirubin-3′-{O-[2-(4-methylpiperazin-1-yl)ethyl]oxime}dihydrochloride, 6-bromoindirubin-3′-(O-{2-[4-(2-hydroxyethyl)piperazin-1-yl]ethyl}oxime)dihydrochloride, 6-bromoindirubin-3′-(O-{2-[4-(2-methoxyethyl)piperazin-1-yl]ethyl}oxime)dihydrochloride and 6-bromoindirubin-3′-[O-(2-{4-[2-(2-hydroxyethoxy)-ethyl]piperazin-1-yl}ethyl)oxime]dihydrochloride.

In some particular embodiments the GSK3 inhibitor is selected from the group consisting of: 6-bromoindirubin-3′-oxime, 6-bromoindirubin-3′-acetoxime, TWS119, functional analogs or derivatives thereof, and combinations thereof; preferably 6-bromoindirubin-3′-oxime, or functional analogs or derivatives thereof.

6-bromoindirubin-3′-oxime or BIO (CAS No. 667463-62-9) is a member of the class of biindoles that is indirubin substituted at position 6 by a bromo group and in which the keto group at position 3′ has undergone condensation with hydroxylamine to form the corresponding oxime. BIO (IC50 1.5 μM) displays remarkable selective inhibition of GSK3 and can therefore activate the canonical Wnt signaling pathway. Bio inhibits the phosphorylation on Tyr276/216 GSK3a/p activation site and reduces beta-catenin phosphorylation (Meijer L et al., 2003). 3-[[6-(3-aminophenyl)-7H-pyrrolo[2,3-d]pyrimidin-4-yl]oxy]phenol or TWS119 (CAS No. 601514-19-6) is a member of pyrroles with an IC50 of 0.6-7 nM.

By “derivative”, or “functional derivative” it is meant herein a compound that is directly derived from a chemical compound of interest and is structurally similar though non-identical to said compound, and which retains the same biological activity and/or physico-chemical properties.

By “analog”, or “functional analog”, it is meant herein a compound that is not directly derived from a chemical compound of interest and is thus structurally different but exhibits the same biological activity and/or physico-chemical properties.

In some particular embodiments, the GSK3 inhibitor suitable for CD8+ T cell reprogramming to induce CD8+ T cells having enhanced stemness and functional properties is selected from indirubins, in particular indirubin of formula (I) as disclosed herein. In some preferred embodiments, the GSK3 inhibitor is selected from the group consisting of: 6-bromoindirubin-3′-oxime, 6-bromoindirubin-3′-acetoxime, 6-bromo-indirubin, 6-bromo-indirubin-3′methoxime, 6-chloro-indirubin, 6-chloro-indirubin-3′-oxime, 6-chloro-indirubin-3′-acetoxime, 6-iodo-indirubin, 6-iodo-indirubin-3′-oxime, 6-iodo-indirubin-3′-acetoxime, 6-vinylindirubin-3′-oxime, 6-vinylindirubin-3′-acetoxime, 6-fluoroindirubin-3′-oxime, 6-fluoroindirubin-3′-acetoxime, 6-bromo-5-methylindirubin, 6-bromo-5-methylindirubin-3′-oxime, 6-bromo-5-methylindirubin-3′-acetoxime, 5,6-dichloroindirubin, 5,6-dichloroindirubin-3′-oxime, 5,6-dichloroindirubin-3′-acetoxime, 6-bromo-5-nitroindirubin, 6-bromo-5-nitroindirubin-3′-oxime, 6-bromo-5-nitroindirubin-3′-acetoxime, 6-bromo-5-aminoindirubin, 6-bromo-5-aminoindirubin-3′-oxime, 5,6-dibromoindirubin, 6-bromoindirubin-3′-[O-(2-bromoethyl)-oxime], 6-bromoindirubin-3′-[O-(2-hydroxyethyl)-oxime], 6-bromoindirubin-3′-[O-(2,3-dihydroxypropyl)-oxime], 6-bromoindirubin-3′-[0-(N,N-diethylcarbamyl)-oxime], 6-bromoindirubin-3′-[O-(2-dimethylaminoethyl)-oxime], 6-bromoindirubin-3′-[O-(2-diethylaminoethyl)-oxime], 6-bromoindirubin-3′-[O-(2-pyrrolidin-1-ylethyl)oxime], 6-bromoindirubin-3′-[O-(2-morpholin-1-ylethyl)oxime], 6-bromoindirubin-3′-[O-(2-(N,N-(2-hydroxyethyl)aminoethyl)oxime], 6-bromoindirubin-3′-(O-{2-[N-methyl, N-(2,3-dihydroxypropyl)amino]ethyl}oxime], 6-bromoindirubin-3′-[0-(2-piperazine-1-ylethyl)oxime], 6-bromoindirubin-3′-(0-[2-(4-methyl-piperazin-1-yl)ethyl]oxime), 6-bromoindirubin-3′-(O-{2-[4-(2-hydroxyethyl)piperazin-1-yl]ethyl}oxime), 6-bromoindirubin-3′-(O-{2-[4-(2-methoxyethyl)piperazin-1-yl]ethyl}oxime), 6-bromoindirubin-3′-[O-(2-(4-[2-(2-hydroxyethoxy)-ethyl]piperazin-1-yl)ethyl)oxime], 6-bromoindirubin-3′-[O-(2-dimethylaminoethyl)oxime]hydrochloride, 6-bromoindirubin-3′-[O-(2-diethylaminoethyl)oxime]hydrochloride, 6-bromoindirubin-3′-[0-(2-pyrrolidin-1-ylethyl)oxime]hydrochloride, 6-bromoindirubin-3′-[O-(2-morpholin-1-ylethyl)oxime]hydrochloride, 6-bromoindirubin-3′-[O-(2-(N,N-(2-hydroxyethyl)aminoethyl)oxime], 6-bromoindirubin-3′-(O-{2-[N-methyl, N-(2,3-dihydroxypropyl)amino]ethyl}oxime]hydrochloride, 6-bromoindirubin-3′-[O-(2-piperazine-1-ylethyl)oxime]dihydrochloride, 6-bromoindirubin-3′-{O-[2-(4-methylpiperazin-1-yl)ethyl]oxime}dihydrochloride, 6-bromoindirubin-3′-(O-{2-[4-(2-hydroxyethyl)piperazin-1-yl]ethyl}oxime)dihydrochloride, 6-bromoindirubin-3′-(O-{2-[4-(2-methoxyethyl)piperazin-1-yl]ethyl}oxime)dihydrochloride and 6-bromoindirubin-3′-[O-(2-{4-[2-(2-hydroxyethoxy)-ethyl]piperazin-1-yl}ethyl)oxime]dihydrochloride; preferably 6-bromoindirubin-3′-oxime and 6-bromoindirubin-3′-acetoxime.

In some particular embodiments, the GSK3 inhibitor suitable for CD8+ T cell reprogramming to enhance effector CD8+ T cells is selected from the group consisting of: Anilinomaleimides; Benzazepinones; Aminopyrimidines, Oxindoles; Pyrazines and Oxazole-carboxamides; preferably selected from the group consisting of: Benzazepinones; Oxindoles; Pyrazines and Oxazole-carboxamides. In some preferred embodiments, the GSK3 inhibitor is selected from the group consisting of: Alsterpaullone, CHIR 98014, SU9516, AZD2858, SB-216763, PF-04802367, functional analogs or derivatives thereof, and combinations thereof; preferably Alsterpaullone, SU9516, AZD2858, PF-04802367, functional analogs or derivatives thereof, and combinations thereof.

In some particular embodiments, the GSK3 inhibitor suitable for CD8+ T cell reprogramming to repress effector CD8+ T cells is selected from the group consisting of: Anilinomaleimides and Bisindolylmaleimides; Pyrroloazepines; Flavones; Pyrrolopyrazines; Pyrrolopyrimidines and Phenylaminopyrimidines; Thiazoles; and Thiadiazolidinones. In some preferred embodiments, the GSK3 inhibitor is selected from the group consisting of: SB-415286, 10Z-Hymenialdisine, Flavopiridol hydrochloride hydrate, Aloisine A, AR-A014418, Staurosporine, GF 109203X, CGP 60474, TWS119, NP031112, functional analogs or derivatives thereof, and combinations thereof.

The method of the invention uses an amount of GSK3 inhibitor and incubation time with the CD8+ T cells that are effective to modulate the Wnt/TCF-1 and/or mTORC signaling pathways following treatment of the population of CD8+ T cells. In some embodiments, a GSK3 inhibitor suitable for CD8+ T cell reprogramming is used to induce CD8+ T cells having enhanced stemness and functional properties. In some particular embodiments, the GSK3 inhibitor increases the level of expression of TCF-1, CCR7, and/or CD27 in the population of CD8+ T cells, in the absence of further stimulation. In some particular embodiments, exposure to the GSK3 inhibitor followed by TCR stimulation, increases the level of expression of CD127, increases the level of activation of mTORC2, and/or decreases the level of activation of mTORC1 in the population of CD8+ T cells. The level of expression of TCF-1, CCR7, CD27, CD127 and the level of activation of mTORC (mTORC1, mTORC2) may be determined according to the routine techniques, well-known of the person skilled in the art as disclosed herein. In some particular embodiments, exposure to the GSK3 inhibitor followed by TCR stimulation, enhances the survival, polyfunctionality, proliferation capacity, metabolic plasticity, response to antigen, response to γ-chain cytokines, response to immune check-point modulators, cytotoxic effect, and/or reduces mTORC1-dependency of the population of CD8+ T cells. In some particular embodiments, exposure to the GSK3 inhibitor, followed by antigen stimulation, increases the frequency of TNF-α+, IFN-γ+, IL-2+ and granzyme B+CD8+ T cells, and cell survival in the population of CD8+ T cells. These properties may be measured according to the routine techniques, well-known of the person skilled in the art as disclosed herein.

In some embodiments, a GSK3 inhibitor suitable for CD8+ T cell reprogramming is used to enhance effector CD8+ T cells. In some particular embodiments, exposure to the GSK3 inhibitor followed by TCR stimulation increases the frequency of CD127−T-bet+; LAG-3+PD-1+; HLA-DR+CD38+; Effector memory (TEM) and/or Terminal effector (TTE) cells in the population of CD8+ T cells. In some particular embodiments, exposure to the GSK3 inhibitor followed by TCR stimulation enhances polyfunctionality of the population of CD8+ T cells characterized by a higher frequency of TNF-α+ and IFN-γ+CD8+ T cells and enhances activation of mTORC1 and mTORC2 pathways characterized by a higher frequency of pS6⁺pAKT⁺CD8+ T cells. These properties may be measured according to the routine techniques, well-known of the person skilled in the art as disclosed herein.

In some embodiments, a GSK3 inhibitor suitable for CD8+ T cell reprogramming is used to repress effector CD8+ T cells. In some particular embodiments, exposure to the GSK3 inhibitor followed by TCR stimulation reduces (or decreases) the frequency of CD127− T-bet+; LAG-3+PD-1+; HLA-DR+CD38+; Effector memory (TEM) and/or Terminal effector (TTE) cells in the population of CD8+ T cells. In some particular embodiments, exposure to the GSK3 inhibitor followed by TCR stimulation reduces polyfunctionality of the population of CD8+ T cells characterized by a lower frequency of TNF-α+ and IFN-γ+CD8+ T cells and reduces activation of mTORC1 and mTORC2 pathways characterized by a lower frequency of pS6⁺pAKT⁺CD8+ T cells. These properties may be measured according to the routine techniques, well-known of the person skilled in the art as disclosed herein.

Therefore, a person skilled in the art can easily determine the amount of GSK3 inhibitor and incubation time to use in the method of the invention.

In some embodiments, the GSK3 inhibitor is used at a concentration of from 1 to 5 μM; preferably about 3 μM. In some embodiments, the GSK3 inhibitor is incubated with the population of CD8+ T cells for 6 to 16 hours, preferably around 12 hours.

In some preferred embodiments, the CD8+ T cells of the population are resting. As used herein, “resting CD8+ T cells” refers to CD8+ T cells maintained in quiescent state, e.g. without T cell receptor (TCR) stimulation. As used herein, “CD8+ T cells activation” or “activated CD8+ T cells” refers to CD8+ T cells comprising TCR stimulation. CD8+ T cells become activated following stimulation of the TCR with the antigen (antigen-specific stimulation) or a TCR binding compound such as anti-CD3 antibody, preferably combined with anti-CD28 antibody (polyclonal stimulation) as disclosed in the examples. Therefore, resting CD8+ T cells are distinct from activated CD8+ T cells.

In some embodiments, the population of CD8+ T cells is in culture. The culture comprises appropriate medium for CD8+ T cells as known in the art for cell culture. For example, the medium is RPMI-1640 supplemented with glutamine, fetal calf serum and antibiotics as disclosed in the examples. The culture is usually maintained at 37° C. with 5% CO₂. In some embodiments, the CD8+ T cells are cultured at a density of 2×10⁶ cells/mL or higher. In some embodiments, the CD8+ T cells are cultured at a density of 2×10⁶ cells/mL lower.

In some embodiments, the cells are further rinsed to eliminate the GSK3 inhibitor from the reprogrammed CD8+ T cells.

In the various embodiments, the reprogrammed CD8+ T cells have enhanced stemness and functional properties or a modulation (enhancement or repression) of effector cells as disclosed in the examples. In particular, the reprogrammed CD8+ T cells have enhanced survival, polyfunctionality, proliferation capacity, metabolic plasticity, response to antigen, response to γ-chain cytokines, response to immune check-point modulators, cytotoxic effect, and/or less mTORC1-dependency as disclosed in the examples.

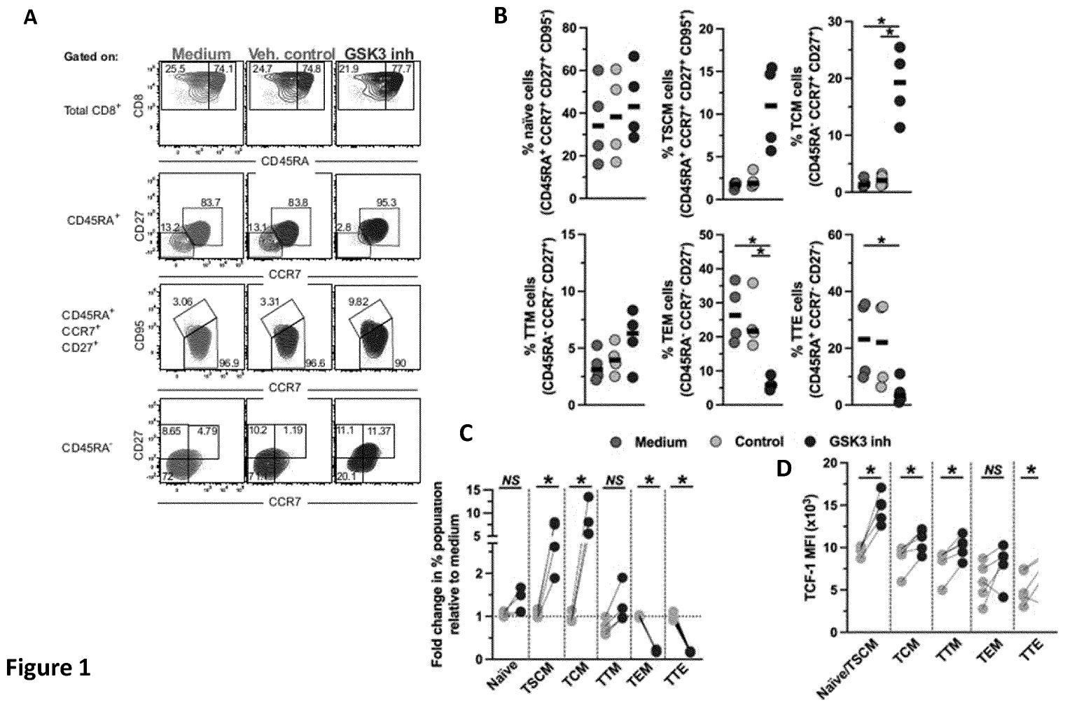

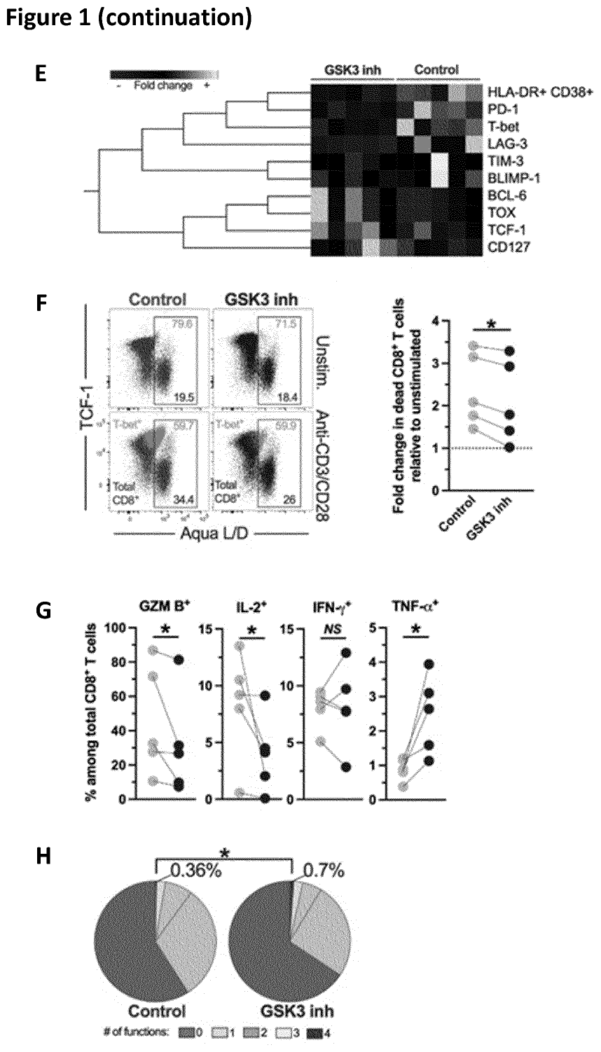

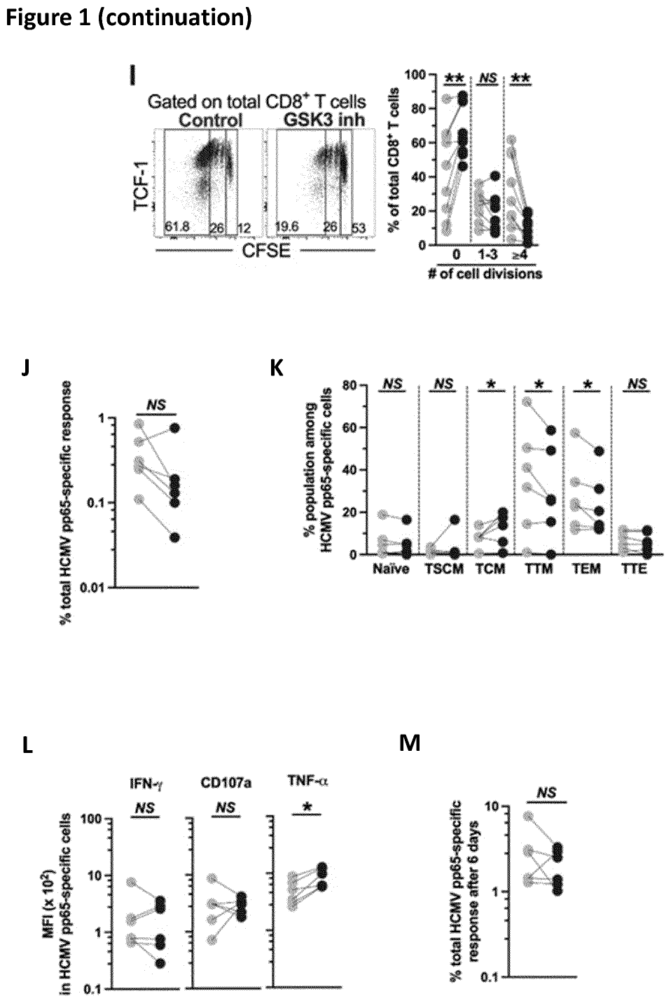

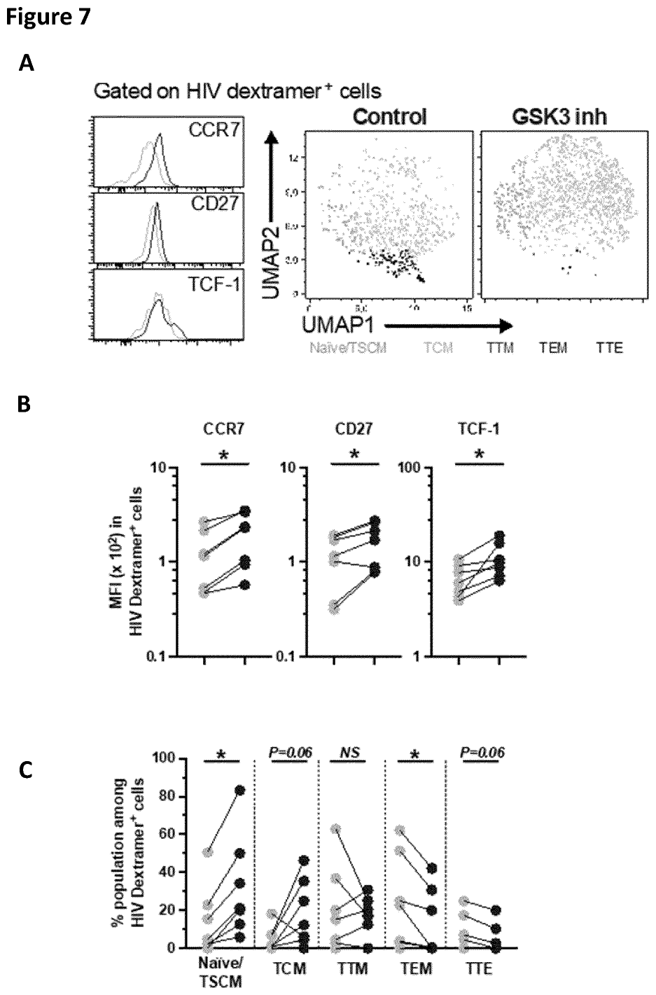

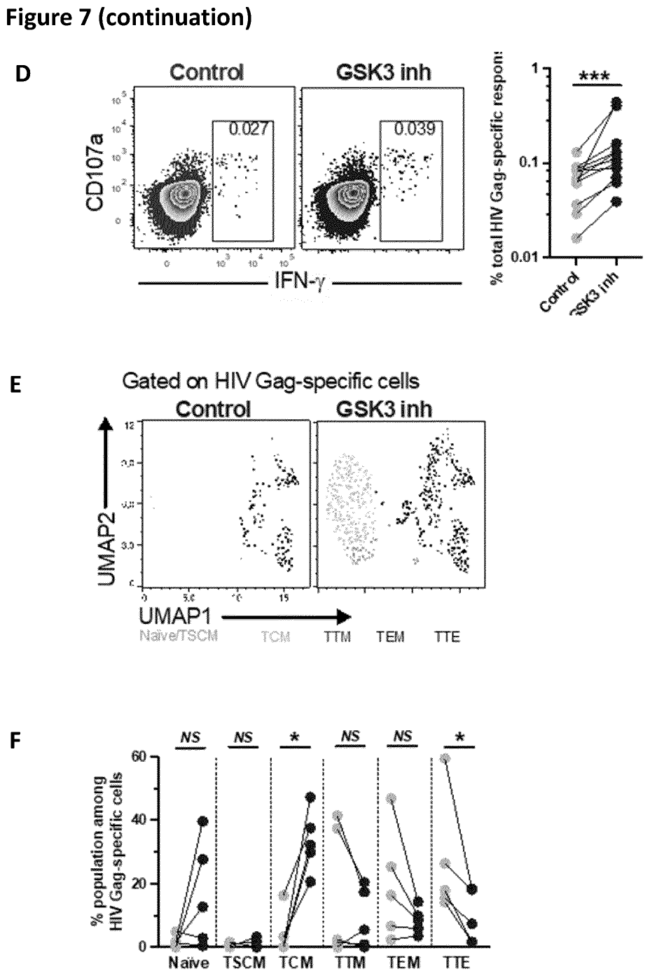

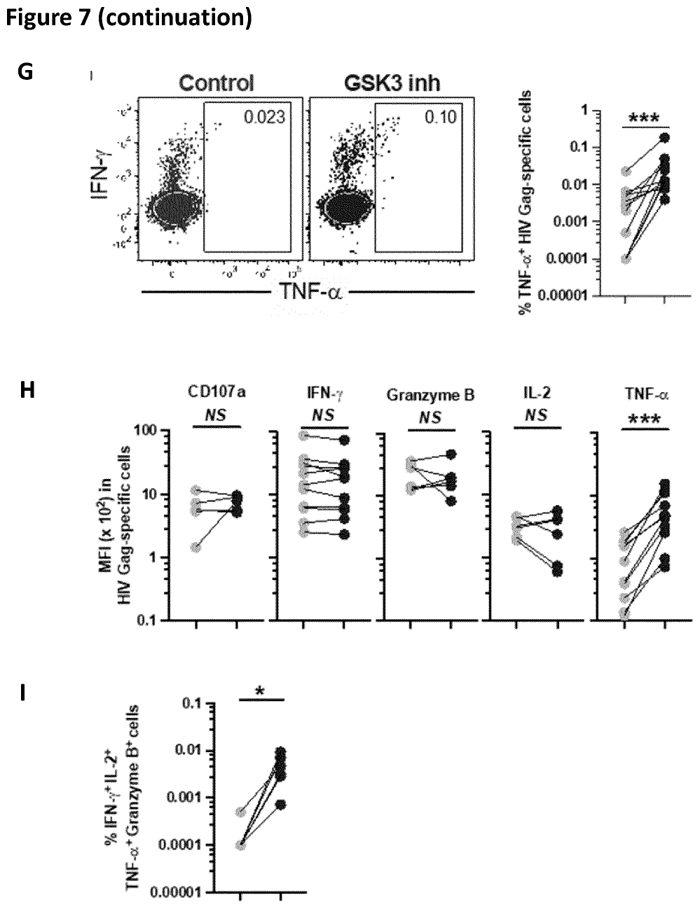

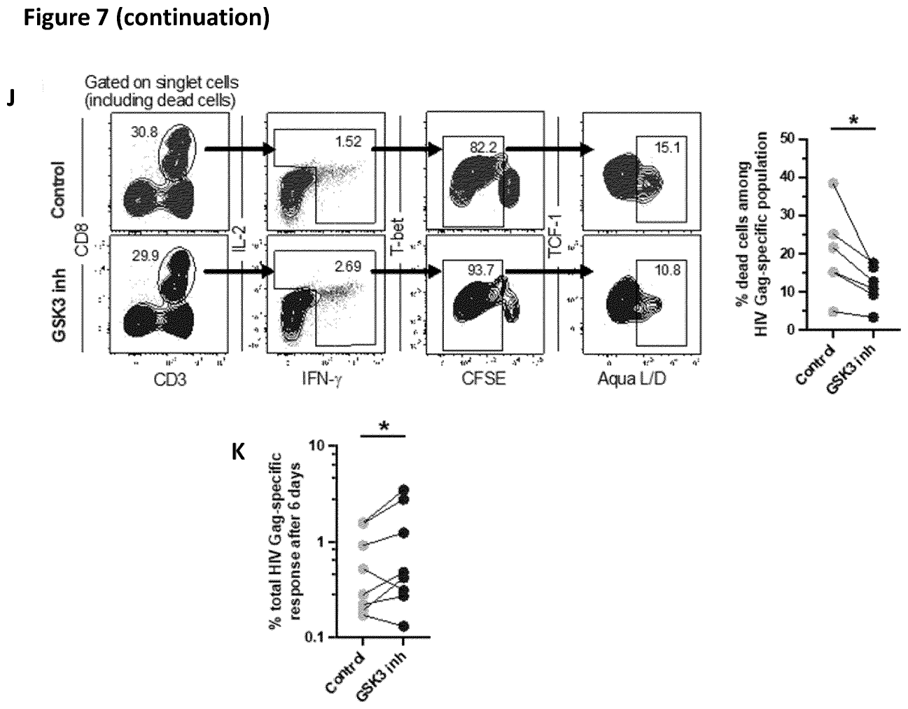

The survival capacity of the reprogrammed CD8+ T cells may be determined by measuring the viability of reprogrammed CD8+ T cells after their activation using standard cell viability assays that are known in the art and disclosed in the examples. For example, reprogrammed CD8+ T cells are stimulated with the antigen for 6 days or with anti-CD3/CD28 antibodies for 48 hrs. The cell viability assay may use a dye, in particular a fluorescent dye that distinguishes live cells from dead cells such as LIVEDEAD Fixable Aqua Dead Cell Stain used in the examples. Measuring the expression level of CD127, which is critical for CD8+ T cell survival is considered as an indicator of CD8+ T cell survival potential. CD127 expression level may be measured by flow cytometry analysis as disclosed herein. Non-reprogrammed and non-stimulated CD8+ T cells are used as control. Reprogrammed CD8+ T cells have enhanced survival compared to non-reprogrammed CD8+ T cells as shown in the examples (FIG. 1F, FIG. 7J, FIG. 7K).

The proliferation capacity of the reprogrammed CD8+ T cells may be determined after their activation using standard cell proliferation assays such as CSFE (Carboxyfluorescein succinimidyl ester) dilution assay disclosed in the examples. For example, reprogrammed CD8+ T cells are stimulated with anti-CD3/CD28 antibodies for 48 hrs followed by wash and survival for 5 days and restimulation with anti-CD3/CD28 antibodies for 48 hrs. Non-reprogrammed CD8+ T cells are used as control. Reprogrammed CD8+ T cells have the capacity to execute 1-3 division cycles but are prevented from excessive proliferation (≥4 cell division) over the period of the test (6 days) and maintain a higher proportion of quiescent cells compared to non-reprogrammed CD8+ T cells as shown in the examples (FIG. 1I).

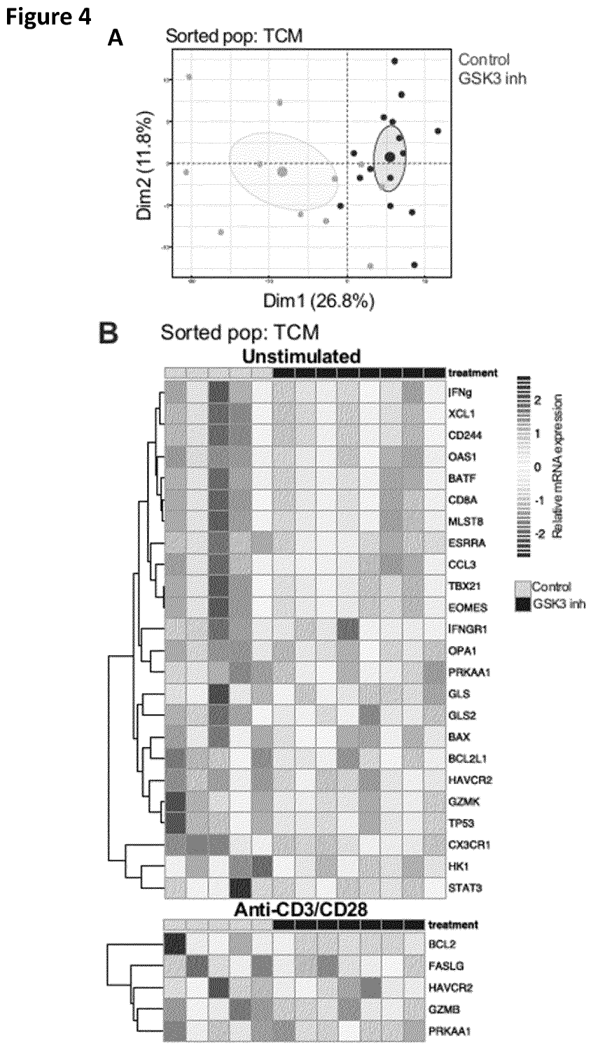

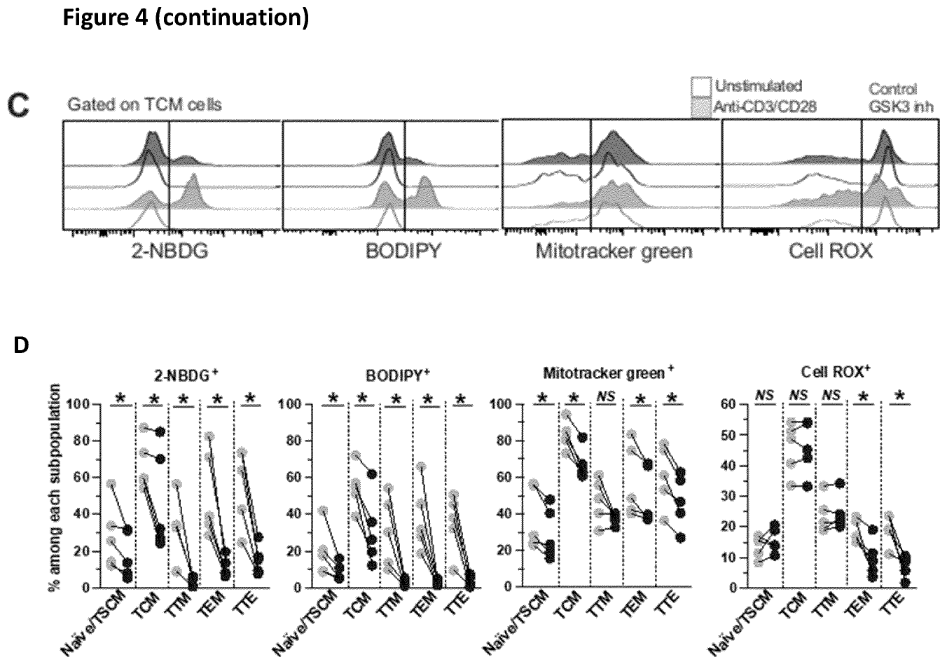

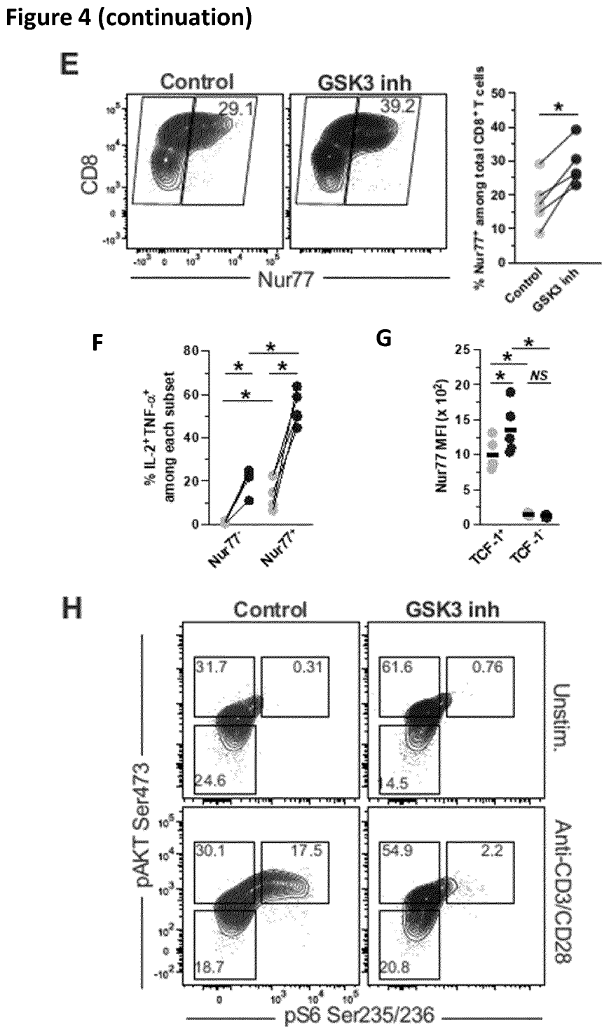

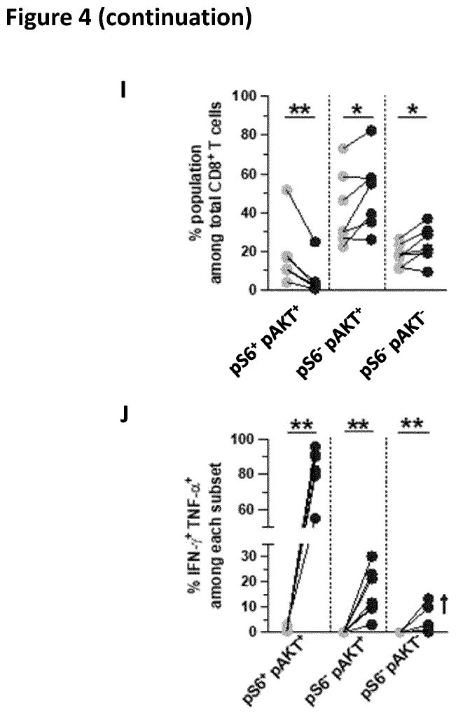

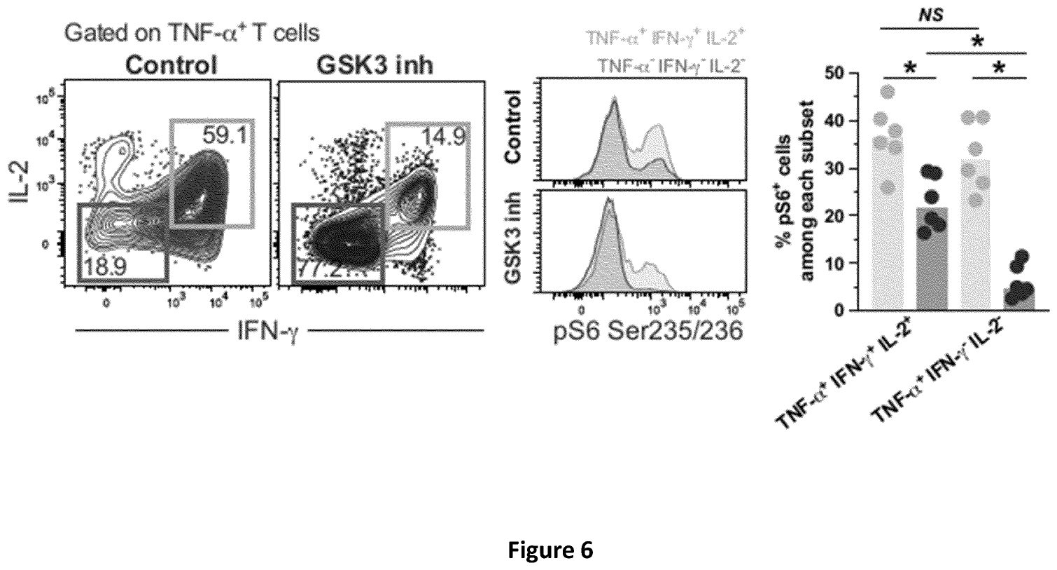

Polyfunctionality refers to the ability of CD8+ T cells to produce several soluble or cytotoxic factors and cytolytic granules (degranulation) after their activation. For example, polyfunctionality may be evaluated by stimulating reprogrammed CD8+ T cells with anti-CD3/CD28 antibodies for 48 hrs and measuring the frequency of CD8+ T cells expressing one to four of granzyme B, IL-2, IFN-γ and TNF-α by cytometry analysis as disclosed herein. Non-reprogrammed CD8+ T cells are used as control. Reprogrammed CD8+ T cells contain a higher frequency of TNF-α+, IFN-γ+ cells; TNF-α+, IFN-γ+, IL-2+ cells; and TNF-α+, IFN-7+, IL-2+, granzyme B+ cells as shown in the examples (FIG. 1H, FIG. 4F, FIG. 7I, FIG. 6).

The antigen response of the reprogrammed CD8+ T cells may be evaluated by stimulating the reprogrammed CD8+ T cells with the antigen, for example for 6 hours, and determining the frequency of antigen-specific CD8+ T cells among CD8+ T cell subsets (naïve, TSCM, TCM, TTM, TEM and TTE) by flow cytometry assisted cell sorting as disclosed in the examples. Non-reprogrammed CD8+ T cells are used as control. Reprogrammed CD8+ T cells contain a higher frequency of antigen-specific CD8+ T cells that are enriched in TCSM and/or TCM cells (FIGS. 7E and F). The enrichment in TSCM and/or TCM does not affect the effector-like functions of the reprogrammed antigen-specific CD8+ T cells such as IFN-γ and CD107a (degranulation marker) expression and their expansion capacity (FIGS. 1L and 1M). Moreover, reprogrammed antigen-specific T cells have a higher production of TNF-α in terms of frequency (FIG. 7G) and intensity of expression (FIG. 7H). The reprogrammed antigen-specific T cells have a higher survival relative to non-reprogrammed antigen-specific CD8+ T cells (FIG. 7J, 7K) as shown in the examples. The results with HIV-specific CD8+ T cells from non-controllers indicate that reprogramming of antigen-specific CD8+ T cells promotes a quantitatively and qualitatively superior response to antigen stimulation, reflected in higher functionality, survival, and expansion capacity, which are features observed in cells from natural HIV-1 controllers.

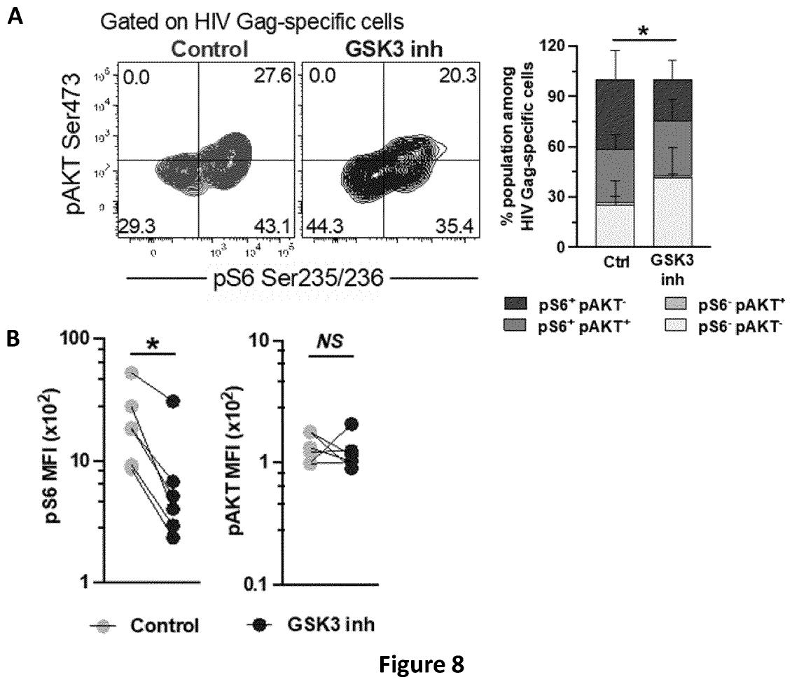

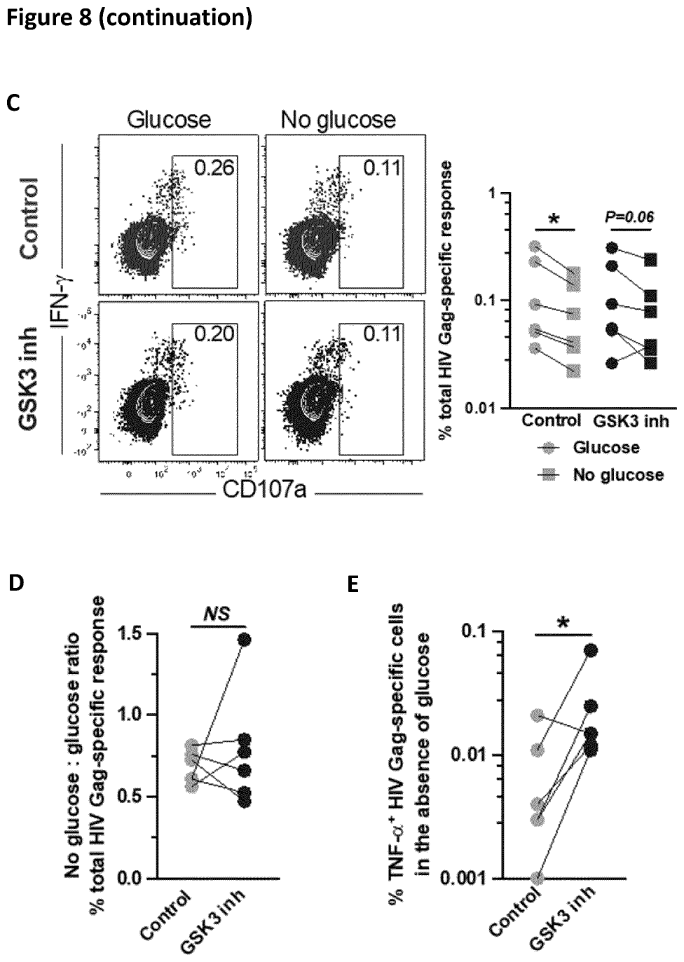

The metabolic plasticity of the reprogrammed CD8+ T cells may be evaluated by stimulating the reprogrammed CD8+ T cells with the antigen, for example for 6 hours, in the absence or presence of nutrients (for instance glucose) and determining the frequency of antigen-specific CD8+ T cells expressing TNF-α by cytometry analysis as disclosed herein. Non-reprogrammed CD8+ T cells are used as control. Reprogrammed antigen-specific CD8+ T cells maintain higher production of TNF-α despite glucose deprivation as shown in the examples (FIG. 8E). The results with HIV-specific CD8⁺ T cells from non-controllers indicate that reprogramming of antigen-specific CD8+ T cells, promotes metabolic plasticity and decreases metabolic restrictions, favoring their strong effector function.

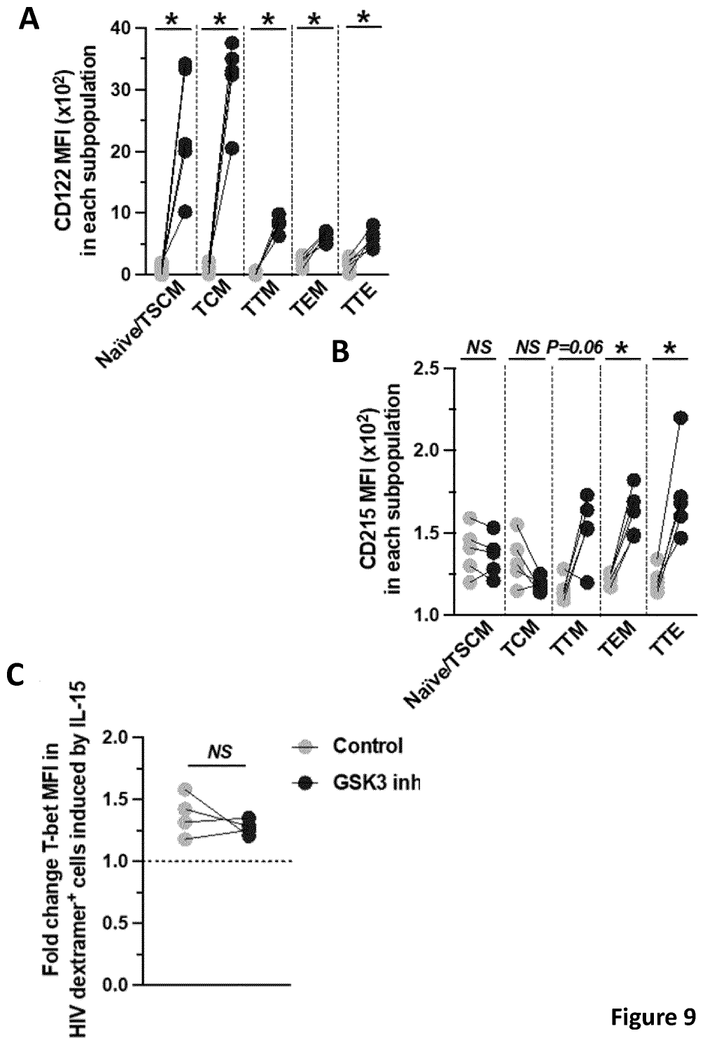

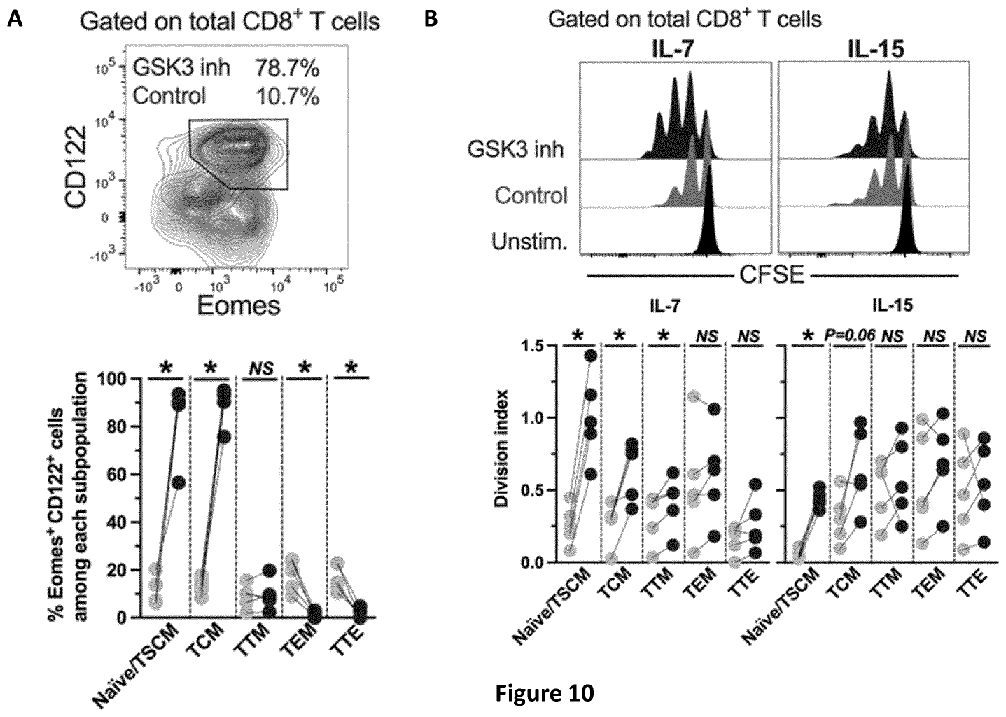

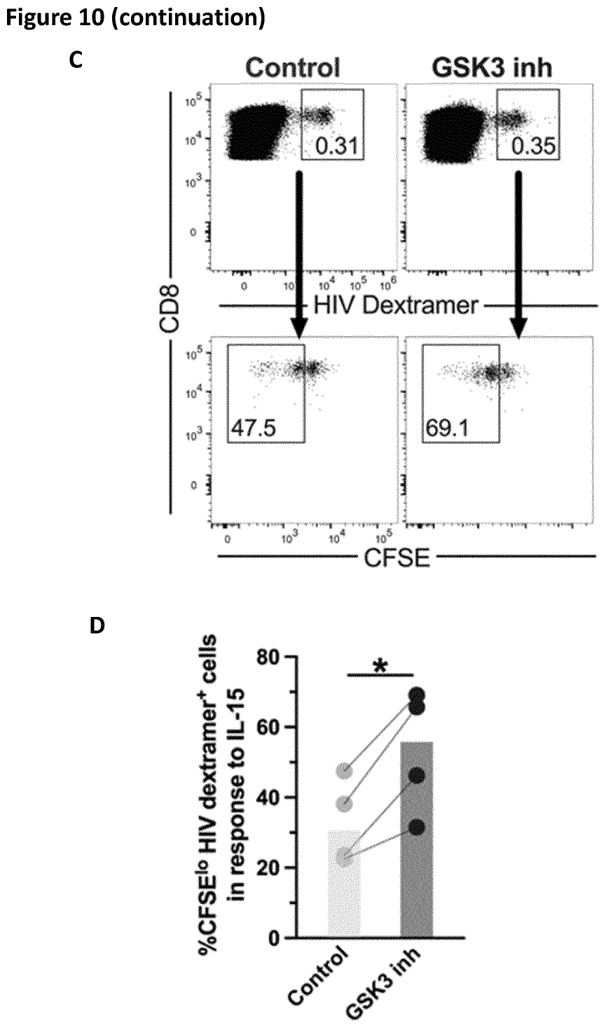

The response to γ-chain cytokines is required for CD8+ T cells self-renewal and long-term maintenance of memory T cells. Reprogrammed CD8⁺ T cells are characterized by higher levels of IL-7 and IL-15 receptors: CD127 (IL-7Rα chain); CD122 (IL-2Rβ chain, component of the IL-15 receptor) and CD215 (IL-15Rα chain) as shown in FIGS. 9A & B. The response to γ-chain cytokines may be evaluated by measuring the proliferation of reprogrammed CD8+ T cell after stimulation with IL-7 or IL-15 using standard cell proliferation assays as disclosed herein. Non-reprogrammed CD8+ T cells are used as control. Reprogrammed CD8+ T cells have an augmented proliferation in response to both cytokines which is higher in naïve/TSCM and TCM cells than in more differentiated cells as shown in FIG. 10B. Moreover, reprogrammed HIV-specific CD8⁺ T cells from non-controllers have an improved proliferative response to IL-15 (FIGS. 10C and D).

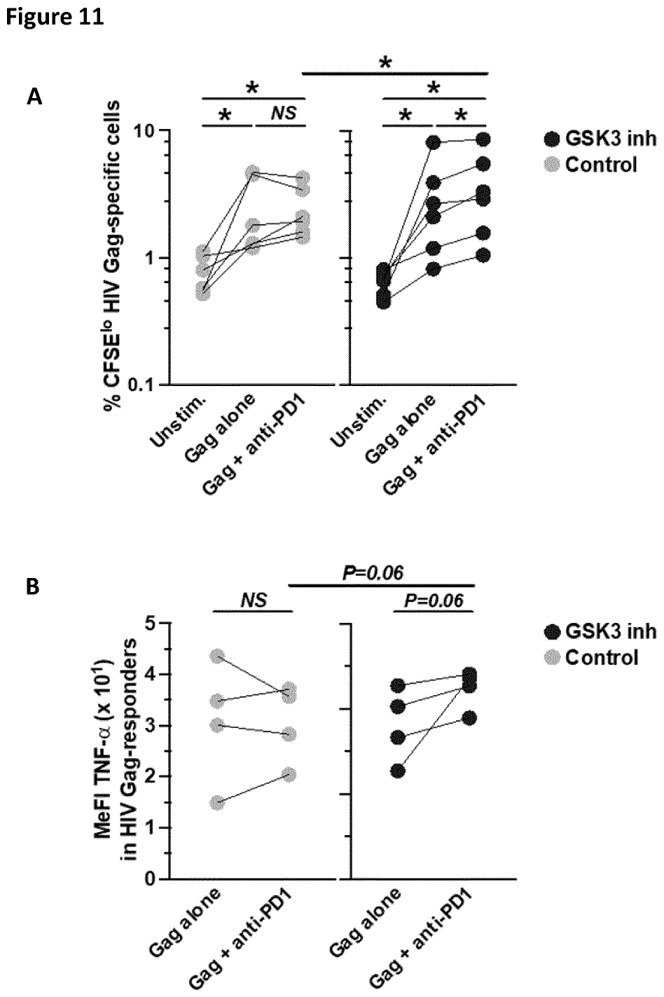

The response to immune-check point modulators of the reprogrammed CD8+ T cells may be evaluated by stimulating the reprogrammed CD8⁺ T cells with the antigen for example for 6 days, in the presence or absence of an anti-PD1 blocking antibody, and determining the proliferation and TNF-α production of the reprogrammed antigen-specific CD8+ T cells as disclosed herein. Non-reprogrammed CD8+ T cells are used as control. The reprogrammed CD8+ T cells have an enhanced response to immune-check point inhibitors, in particular PD-1 inhibitors as disclosed in the examples (FIG. 11).

The cytotoxic capacity of the reprogrammed CD8+ T cells may be determined by measuring the cytotoxic activity of reprogrammed CD8+ T cells after their activation using standard cytotoxic assays such that are known in the art. Non-reprogrammed CD8+ T cells are used as control.

The less mTORC1-dependency of the reprogramed CD8+ T cells may be evaluated by stimulating the reprogrammed CD8⁺ T cells with anti-CD3/CD28 and analyzing the phosphorylation of ribosomal S6 (pS6 Ser235/236) and AKT (pAKT Ser473) proteins, markers of activation of mTORC1 and mTORC2, respectively as disclosed in the examples. Non-reprogrammed and non-stimulated CD8+ T cells are used as control. Reprogrammed CD8+ T cells have less up-regulation of pS6 and higher levels of pS6⁻ pAKT⁺ cells confirming that reprogrammed CD8+ T cells maintain a relative metabolic quiescence supported preferentially by activation of the mTORC2 pathway as shown in the examples (FIGS. 4H and 4I). The results further show that reprogrammed HIV-specific CD8+ T cells from non-controllers had lower proportions of pS6+ cells and pS6⁺pAKT⁻ cells and no difference for pAKT+ compared to non-reprogrammed HIV-specific CD8⁺ T cells (FIGS. 8A and B). These data indicate that reprogrammed HIV-specific CD8⁺ T cells had a diminished dependency on mTORC1 for supporting a strong antigen-induced response, while preserving mTORC2 activation to exert their functions.

In some embodiments, the reprogrammed CD8+ T cells have enhanced expression level of TCF-1 in the absence of further stimulation; preferably further having enhanced expression levels of CCR7 and CD27 in the absence of further stimulation, compared to non-reprogrammed CD8+ T cells.

In some particular embodiments, the reprogrammed CD8+ T cells have enhanced expression levels of TCF-1, CD127, CD122, CD215, and/or TNF-α and optionally further BCL-6, and/or TOX in response to activation (TCR stimulation), and/or enhanced proliferation in response to stimulation with IL-7 and/or IL-15 compared non-reprogrammed CD8+ T cells. In some particular embodiments, the reprogrammed CD8+ T cells have decreased expression levels of HLA-DR, CD38, PD-1, LAG-3, TIM-3, T-bet, and/or BLIMP-1 in response to activation. In some preferred embodiments, the reprogrammed CD8+ T cells have enhanced expression levels of TCF-1, CD127 and TNF-α in response to activation: more preferably, the reprogrammed CD8+ T cells further have enhanced expression levels of CD122 and/or CD215 in response to activation, and/or enhanced proliferation in response to stimulation with IL-7 and/or IL-15 compared non-reprogrammed CD8+ T cells.

In some particular embodiments, the reprogrammed CD8+ T cells have an enhanced response to antigen, characterized by: (i) a higher frequency of antigen-specific CD8+ T cells that are enriched in TSCM and TCM cells; (ii) higher frequency of antigen-specific CD8+ T cells producing cytokines, such as TNF-α and/or (iii) antigen-specific CD8+ T cells having higher survival compared non non-reprogrammed CD8+ T cells.

In some particular embodiments, the reprogrammed CD8+ T cells have an enhanced polyfunctionality characterized by a higher frequency of TNF-α+ and IFN-γ+ cells; preferably further IL-2+; more preferably further granzyme B+ compared to unreprogrammed CD8+ T cells.

In some particular embodiments, the reprogrammed CD8+ T cells have an enhanced proliferation capacity, characterized by a lower frequency of cells executing more than 4 cell division over a period of 6 days and a higher frequency of quiescent cells compared to non-reprogrammed CD8+ T cells.

In some particular embodiments, the reprogrammed CD8+ T cells have less mTORC1-dependency in response to activation, characterized by a lower frequency of pS6+ cells and/or higher frequency of pS6⁻ pAKT⁺ cells compared to non-reprogrammed CD8⁺ T cells. In some preferred embodiments, the reprogrammed CD8⁺ T cells have a lower frequency of pS6+ among TNF-α+ cells.

In some particular embodiments, the reprogrammed CD8+ T cells have a reduced anabolic metabolism in response to activation, characterized by reduced glucose uptake, lipid uptake, oxygen species production, and/or mitochondrial mass augmentation compared to non-reprogrammed CD8+ T cells. In some preferred embodiments, the reprogrammed CD8+ T cells have a higher frequency of Nur77+ cells; preferably further TNF-α+ and IL-2+.

In some particular embodiments, the reprogrammed CD8+ T cells have an enhanced metabolic plasticity characterized by maintenance of a higher production of TNF-α despite glucose deprivation, compared to non-reprogrammed CD8+ T cells.

In some particular embodiments, the reprogrammed CD8+ T cells comprise a higher proportion of memory CD8+ T cells with stemness, preferably of the Stem cell memory (TSCM) and Central memory (TCM) subsets; preferably, the reprogrammed CD8+ T cells further comprise a lower proportion of the more differentiated, effector (TTE) and effector memory (TEM) CD8+ T cells compared to non-reprogrammed CD8+ T cells.

In some particular embodiments, the proportion of memory CD8+ T cells with stemness is increased by at least 2-fold, preferably 2-fold to up to 10-fold (2, 3, 4, 5, 6, 7, 8, 9, 10), in particular by at least 5-fold in the reprogrammed CD8+ T cells compared to non-reprogrammed CD8+ T cells. As shown in the examples, the reprogrammed CD8+ T cells can comprise about 10% of TSCM, 20% of TCM, 6% of TEM and 3% of TTE. In contrast the non-reprogrammed CD8+ T cells comprise only about 2% of TCM, 2% of TSCM, 22% of TEM and 20% of TTE (see FIG. 1B).

In the various embodiments, the reprogrammed memory CD8+ T cells with stemness have enhanced stemness and functional properties. In particular, the reprogrammed memory CD8+ T cells with stemness have enhanced self-renewal, long-term maintenance and response to antigen compared to non-reprogrammed memory CD8+ T cells with stemness. In some particular embodiments, the reprogrammed memory CD8+ T cells with stemness have a higher frequency of antigen-specific CD8+ T cells and an enhanced proliferation in response to IL-7 and/or IL-15 compared to non-reprogrammed memory CD8+ T cells with stemness.

Examples of GSK3 inhibitors capable of inducing reprogrammed memory CD8+ T cells with enhanced stemness and functional properties according to the present disclosure are disclosed in the examples and include indirubin compounds, in particular indirubin of formula (I) as disclosed herein such as BIO and BIO-acetoxime.

Examples of GSK3 inhibitors capable of inducing reprogrammed CD8+ T cells having an enhanced polyfunctionality in response to TCR stimulation characterized by a higher frequency of TNF-α+ and IFN-γ+CD8+ T cells compared to non-reprogrammed CD8+ T cells are disclosed in the examples and include in particular compounds selected from the group consisting of: Anilinomaleimides; Benzazepinones; Bis-Indoles, in particular indirubins such as indiubin of formula (I) as disclosed herein; Aminopyrimidines, Oxindoles; Pyrazines and Oxazole-carboxamides; preferably selected from the group consisting of: Benzazepinones; Bis-Indoles; Oxindoles; Pyrazines and Oxazole-carboxamides. In some preferred embodiments, the GSK3 inhibitor is selected from the group consisting of: Alsterpaullone, BIO and BIO-Acetoxime, CHIR 98014, SU9516, AZD2858, SB-216763, PF-04802367, functional analogs or derivatives thereof, and combinations thereof; preferably Alsterpaullone, BIO and BIO-Acetoxime, SU9516, AZD2858, PF-04802367, functional analogs or derivatives thereof, and combinations thereof.

In some particular embodiments, the reprogrammed CD8+ T cells have a reduced polyfunctionality in response to TCR stimulation characterized by a lower frequency of TNF-α+ and IFN-γ+CD8+ T cells compared to non-reprogrammed CD8+ T cells. Examples of GSK3 inhibitors having these properties are disclosed in the examples and include in particular compounds selected from the group consisting of: ions (competition with Mg2+); Anilinomaleimides and Bisindolylmaleimides; Pyrroloazepines; Flavones; Pyrrolopyrazines; Pyrrolopyrimidines and Phenylaminopyrimidines; Thiazoles; and Thiadiazolidinones. In some preferred embodiments, the GSK3 inhibitor is selected from the group consisting of: SB-415286, Lithium carbonate, 10Z-Hymenialdisine, Flavopiridol hydrochloride hydrate, Aloisine A, AR-A014418, Staurosporine, GF 109203X, CGP 60474, TWS119, NP031112, functional analogs or derivatives thereof, and combinations thereof.

In some particular embodiments, the reprogrammed CD8+ T cells have a high activation of mTORC1 and mTORC2 pathways in response to TCR stimulation, characterized by a higher frequency of pS6⁺pAKT⁺CD8+ T cells compared to non-reprogrammed CD8+ T cells. Examples of GSK3 inhibitors having these properties are disclosed in the examples and include in particular compounds selected from the group consisting of: ions (competition with Mg2+); Anilinomaleimides; Benzazepinones; Aminopyrimidines, Oxindoles; Pyrazines and Oxazole-carboxamides; preferably selected from the group consisting of: ions (competition with Mg2+); Benzazepinones; Bis-Indoles; Oxindoles; Pyrazines and Oxazole-carboxamides. In some preferred embodiments, the GSK3 inhibitor is selected from the group consisting of: Alsterpaullone, Lithium carbonate, CHIR 98014, SU9516, AZD2858, SB-216763, PF-04802367, functional analogs or derivatives thereof, and combinations thereof; preferably Alsterpaullone, Lithium carbonate, SU9516, AZD2858, PF-04802367, functional analogs or derivatives thereof, and combinations thereof.

In some particular embodiments, the reprogrammed CD8+ T cells have a low activation of mTORC1 and mTORC2 pathways in response to TCR stimulation, characterized by a lower frequency of pS6⁺pAKT⁺CD8+ T cells compared to non-reprogrammed CD8+ T cells. Examples of GSK3 inhibitors having these properties are disclosed in the examples and include in particular compounds selected from the group consisting of: Bis-Indoles, in particular indirubins such as indiubin of formula (I) as disclosed herein; Anilinomaleimides and Bisindolylmaleimides; Pyrroloazepines; Flavones; Pyrrolopyrazines; Pyrrolopyrimidines and Phenylaminopyrimidines; Thiazoles; and Thiadiazolidinones. In some preferred embodiments, the GSK3 inhibitor is selected from the group consisting of: BIO and BIO-Acetoxime, SB-415286, 10Z-Hymenialdisine, Flavopiridol hydrochloride hydrate, Aloisine A, AR-A014418, Staurosporine, GF 109203X, CGP 60474, TWS119, NP031112, functional analogs or derivatives thereof, and combinations thereof.

In some more particular embodiments, the reprogrammed CD8+ T cells have: (i) a reduced polyfunctionality in response to TCR stimulation characterized by a lower frequency of TNF-α+ and IFN-γ+CD8+ T cells and (ii) a low activation of mTORC1 and mTORC2 pathways in response to TCR stimulation, characterized by a lower frequency of pS6⁺pAKT⁺CD8+ T cells, compared to non-reprogrammed CD8+ T cells. Examples of GSK3 inhibitors having these properties are disclosed in the examples and include in particular compounds selected from the group consisting of: Anilinomaleimides and Bisindolylmaleimides; Pyrroloazepines; Flavones; Pyrrolopyrazines; Pyrrolopyrimidines and Phenylaminopyrimidines; Thiazoles; and Thiadiazolidinones. In some preferred embodiments, the GSK3 inhibitor is selected from the group consisting of: SB-415286, 10Z-Hymenialdisine, Flavopiridol hydrochloride hydrate, Aloisine A, AR-A014418, Staurosporine, GF 109203X, CGP 60474, TWS119, NP031112, functional analogs or derivatives thereof, and combinations thereof.

In some more particular embodiments, the reprogrammed CD8+ T cells have: (i) an enhanced polyfunctionality in response to TCR stimulation characterized by a higher frequency of TNF-α+ and IFN-γ+CD8+ T cells and (ii) a high activation of mTORC1 and mTORC2 pathways in response to TCR stimulation, characterized by a higher frequency of pS6⁺pAKT⁺CD8+ T cells, compared to non-reprogrammed CD8+ T cells. Examples of GSK3 inhibitors having these properties are disclosed in the examples and include in particular compounds selected from the group consisting of: Anilinomaleimides; Benzazepinones; Aminopyrimidines, Oxindoles; Pyrazines and Oxazole-carboxamides; preferably selected from the group consisting of: Benzazepinones; Oxindoles; Pyrazines and Oxazole-carboxamides. In some preferred embodiments, the GSK3 inhibitor is selected from the group consisting of: Alsterpaullone, CHIR 98014, SU9516, AZD2858, SB-216763, PF-04802367, functional analogs or derivatives thereof, and combinations thereof; preferably Alsterpaullone, SU9516, AZD2858, PF-04802367, functional analogs or derivatives thereof, and combinations thereof.

In some more particular embodiments, the reprogrammed CD8+ T cells have: (i) an enhanced polyfunctionality in response to TCR stimulation characterized by a higher frequency of TNF-α+ and IFN-γ+CD8+ T cells and (ii) a low activation of mTORC1 and mTORC2 pathways in response to TCR stimulation, characterized by a lower frequency of pS6⁺pAKT⁺CD8+ T cells, compared to non-reprogrammed CD8+ T cells. Examples of GSK3 inhibitors having these properties are disclosed in the examples and include in particular compounds selected from the group consisting of: Bis-Indoles, in particular indirubins such as indiubin of formula (I) as disclosed herein. In some preferred embodiments, the GSK3 inhibitor is selected from the group consisting of: BIO and BIO-Acetoxime, functional analogs or derivatives thereof, and combinations thereof.

In some preferred embodiments, the reprogrammed CD8+ T cells comprise a higher frequency of effector CD8+ T cells, in particular CD127− T-bet+; LAG-3+PD-1+; HLA-DR+CD38+; Effector memory (TEM) and/or Terminal effector (TTE) CD8+ T cells, compared to the non-reprogrammed CD8+ T cells. Examples of GSK3 inhibitors having these properties are disclosed in the examples and include in particular compounds selected from the group consisting of: Thiadiazolidinones, Oxindoles, Aminopyrimidines, Pyrroloazepines and Pyrazines. In some preferred embodiments, the GSK3 inhibitor is selected from the group consisting of: Tideglusib, SU9516, CHIR 98014, 10-hymedianisne, and AZD2858.

In some preferred embodiments, the reprogrammed CD8+ T cells comprise a lower frequency of effector CD8+ T cells, in particular CD127− T-bet+; LAG-3+PD-1+; HLA-DR+CD38+; Effector memory (TEM) and/or Terminal effector (TTE) CD8+ T cells, compared to the non-reprogrammed CD8+ T cells. Examples of GSK3 inhibitors having these properties are disclosed in the examples and include in particular compounds selected from the group consisting of: Phenylamidopyrimidines, Thiazoles and Bisindolylmaleimides. In some preferred embodiments, the GSK3 inhibitor is selected from the group consisting of: CGP60474, ARA014418, and staurosporine.

In the various embodiments, the reprogrammed CD8+ T cells have enhanced antimicrobial, in particular antiviral effect, or enhanced antitumoral effect.

In the various embodiments, the reprogrammed CD8+ T cells have enhanced efficacy for adoptive T cell therapy (ACT). Adoptive T cell therapy is in particular for treating infectious diseases and cancer.

In various embodiments, the method may further comprise isolating, stimulating, expanding, engineering, and/or activating the reprogrammed CD8+ T cells.

In some embodiments, the method further comprises isolating the reprogrammed CD8+ T cells. Routine techniques in the art, such as those set forth in the examples, can be used to select these cells. In preferred embodiments, cells are sorted using well-known methods in the art. In some embodiments, flow cytometry assisted cell sorting or magnetic cell separation are used to isolate particular cell types. Antibodies against any of the markers described herein can be used to achieve isolation, purification, and/or detection of any of the cell markers described herein.

In some embodiments, the method further comprises stimulating the reprogrammed CD8+ T cells with a cytokine or another factor, in particular IL-15. For example, IL-15 can be used at up to 10 ng/mL or higher, for up to 6 days or more. IL-15 improves the proliferation of the reprogrammed CD8+ T cells and maturation of their effector profile as shown in the examples.

In some embodiments, the method further comprises activating the reprogrammed CD8+ T cells. The reprogrammed CD8+ T cells are activated by stimulation of the TCR with the antigen (antigen-specific stimulation) or a TCR binding compound such as anti-CD3 antibody, possibly combined with anti-CD28 antibody (polyclonal stimulation) as disclosed in the examples. The stimulation of the reprogrammed cells with peptides specific for an antigen of interest would help to expand specifically antigen-specific cells. The antigen of interest is preferably a tumoral antigen or microbial antigen such as viral antigen.

In some embodiments, the method does not comprise activating the reprogrammed CD8+ T cells, in particular reprogrammed engineered CD8+ T cells such as reprogrammed CAR-T cells. In some embodiments, the method does not comprise activating the reprogrammed CD8+ T cells with the antigen, preferably wherein the reprogrammed CD8+ T cells are reprogrammed engineered CD8+ T cells such as reprogrammed CAR-T cells. This means that the reprogrammed CD8+ T cells are not activated by stimulation of the TCR with the antigen (antigen-specific stimulation). In some particular embodiments, the method further comprises stimulating the reprogrammed CD8+ T cells with a cytokine or another factor, in particular IL-15 but does not comprise activating the reprogrammed CD8+ T cells; preferably wherein the reprogrammed CD8+ T cells are reprogrammed engineered CD8+ T cells such as reprogrammed CAR-T cells. In some particular embodiments, the method further comprises stimulating the reprogrammed CD8+ T cells with a cytokine or another factor, in particular IL-15 but does not comprise activating the reprogrammed CD8+ T cells with the antigen; preferably wherein the reprogrammed CD8+ T cells are reprogrammed engineered CD8+ T cells such as reprogrammed CAR-T cells.

In some embodiments, the method further comprises engineering the reprogrammed CD8+ T cells. In particular embodiments, the cells are modified to contain a chimeric antigen receptor (CAR). These CARs typically comprise a single-chain binding domain, such as from a monoclonal antibody or nanobody, fused to a transmembrane domain and endodomain that results in the transmission of a signal in response to binding of the binding domain to its target. Examples of CARs are well-known in the art. Such a genetically-engineered receptor, can be used to graft the specificity of a monoclonal antibody or specific receptors for a given antigen onto CD8+ T cells. The engineered T cells comprising CAR are called CAR-T cells. In some particular embodiments, the GSK3 inhibitor is administered to a population of engineered CD8+ T cells, in particular CAR-T cells, to obtained reprogrammed engineered CD8+ T cells, in particular reprogrammed CAR-T cells.

In some embodiments, the method comprises administering the reprogrammed CD8+ T cells to a subject. CD8+ T cells can be autologous or allogenic. Allogenic refers to histocompatible (HLA-compatible) cells. For example, a population of autologous CD8+ T cells obtained from a subject sample is reprogrammed and re-administered to the subject, in particular a patient having an infectious disease or cancer. In some embodiments, a population of allogenic CD8+ T cells obtained from a donor sample is reprogrammed and administered to a recipient subject.

In some embodiments, the method for reprogramming CD8+ T cells, comprises:
a) Providing a population of CD8+ T cells from a donor subject;
b) Administering at least one GSK3 inhibitor to the population of CD8+ T cells to induce reprogrammed CD8+ T cells having enhanced stemness and functional properties or a modulation of effector cells compared to non-reprogrammed CD8+ T cells; and
c) Administering the reprogrammed CD8+ T cells to a recipient subject which may be the donor subject or an histocompatible subject.

In some embodiments, the reprogrammed CD8+ T cells are isolated, stimulated, activated and/or engineered before administration to the subject. In particular embodiments, the reprogrammed CD8+ T cells are isolated before administration to the subject. In particular embodiments, the reprogrammed CD8+ T cells are specific for an antigen of interest, preferably a tumoral antigen or microbial antigen such as viral antigen. In preferred embodiments, the reprogrammed CD8+ T cells are expanded before administration to the subject. In particular embodiments, the reprogrammed CD8+ T cells are stimulated with IL-15 or TCR engagement to expand the CD8+ T cells before administration to the subject; in more particular embodiments, the reprogrammed CD8+ T cells are stimulated with IL-15 to expand the CD8+ T cells before administration to the subject. In particular embodiments, the reprogrammed CD8+ T cells are engineered cells, in particular CAR-T cells. In more particular embodiments, the CAR-T cells are not activated. In more particular embodiments, the CAR-T cells are not activated with the antigen. In more particular embodiments, the CAR-T cells are stimulated with IL-15 to expand the CD8+ T cells before administration to the subject. In some embodiments, step c) comprises infusing the reprogrammed CD8+ T cells to a subject.

### Composition Comprising Reprogrammed CD8+ T Cells and Use in Therapy

Another aspect of the invention relates to the reprogrammed CD8+ T cells produced by the method of the invention as disclosed herein. In particular, the invention relates to a pharmaceutical composition comprising reprogrammed CD8+ T cells produced by the method of the invention as disclosed herein. In some embodiments, the composition is a pharmaceutical composition comprising an effective amount of the reprogrammed CD8+ T cells and a pharmaceutically acceptable vehicle and/or carrier.

By “effective amount”, it is meant herein a therapeutically effective or pharmaceutically effective dose. An effective amount of reprogrammed CD8+ T cells is an amount sufficient to induce a protective response against the disease, in particular a protective response against the tumor in a cancer patient or a protective response against the pathogen in a patient having an infectious disease. The therapeutically effective dose depends upon the composition used, the route of administration, the type of mammal (human or animal) being treated, the physical characteristics of the specific mammal under consideration, concurrent medication, and other factors, that those skilled in the medical arts will recognize.

The pharmaceutical composition is formulated for administration by a number of routes, including but not limited to parenteral and local. The pharmaceutical vehicles and carriers are those appropriate to the planned route of administration, which are well known in the art.

In some embodiments, the composition further comprises another pharmaceutical agent or therapeutic, preferably an anticancer, anti-infectious or immunomodulatory agent.

Another aspect of the invention relates to a method for treatment of a patient in need thereof, comprising:
Reprogramming autologous or allogenic CD8+ T cells according to the reprogramming method of the disclosure; and
Administering the reprogrammed CD8+ T cells to the patient.

The method comprises adoptive T cell therapy which may be combined with another therapy. Adoptive cell therapy (ACT) also called adoptive T cell therapy, adoptive cell transfer, cellular adoptive immunotherapy or T-cell transfer therapy is a type of immunotherapy in which T cells are administered to a patient to help the immune system fight diseases such as cancer and infectious diseases. The T cells used in adoptive cell therapy may be autologous or allogenic T cells and modified T cells such as CAR-T cells. CAR-T cell therapy and hematopoietic stem-cells (HSC) transfer are included in adoptive cell therapy.

Reprogrammed CD8+ T cells with enhanced stemness and functional properties are in particular useful for treatment of infectious diseases or cancer or for hematopoietic stem-cell transplantation. Reprogrammed CD8+ T cells having an enhancement of effector CD8+ T cells are in particular useful for treatment infectious diseases or cancer. Infectious diseases are in particular chronic or recurrent infections. Without being bound by theory, it is believed that reprogrammed cells with stemness as they have multipotency and survival capacities will provide a durable effect, while the reprogrammed cells with effector characteristics may have an immediate short-term enhanced antiviral or antitumor activity. Reprogrammed CD8+ T cells having a repression of effector CD8+ T cells are in particular useful for the treatment of chronic or acute inflammatory diseases, auto-immune diseases, allergic diseases, graft-versus-host disease (GVHD) and graft rejection.

In some embodiments, the method is for treatment of an infectious disease or cancer. In some embodiments, the method is for hematopoietic stem-cell transplantation.

The patient is in need of CD8+ T cells reprogramming to enhance their potential for eliminating diseased cells and provide durable immunosurveillance. In some embodiments, the patient can be immune deficient, immunocompromised, or immune suppressed. In some embodiments, the patient has a dysfunctional immune response to a disease and does not control the disease. In some particular embodiments, the patient has a cancer. In some particular embodiments, the patient has an infectious disease.

As used herein, “cancer” refers to any type of cancer such as carcinomas, lymphomas (Hodgkin and non-Hodgkin), leukemia, sarcomas, mesotheliomas, gliomas, germinomas, choriocarcinomas, neuroblastomas, melanomas and other cancers that may affect various tissues or organs such as with no limitations: breast, prostate, lung, colon, skin, uterus, brain, hematopoietic and reticuloendothelial systems; blood; and lymph nodes. In some embodiments, the cancer is a solid tumor such as with no limitation metastatic melanoma or sarcoma and others.

Infectious diseases include viral, bacterial, fungal and parasitic diseases. In some embodiments, the infectious disease is chronic (persistent or recurrent infection). In some embodiments, the infectious disease is opportunistic. In some embodiments, the infectious disease is a viral disease. Non-limiting examples of viral infections include: HIV/AIDS including AIDS-related complexes; viral hepatitis such as Hepatitis B (HBV), Hepatitis C (HCV) and hepatitis D (HDV); Herpes virus such as HSV-1, HSV-2, EBV, CMV, VZV, HHV6 infections, and retrovirus such as HTLV infections. In some preferred embodiments, the infectious disease is a viral disease selected from the group consisting of: HIV/AIDS, Hepatitis B (HBV), Hepatitis C (HCV), HTLV and CMV infections.

In some embodiments, the method comprises administering a pharmaceutical composition comprising the reprogrammed CD8+ T cells according to the present disclosure.

In some embodiments, the method further comprises administering at least another therapy to the patient, preferably an anticancer, anti-infectious therapy and/or immunotherapy. The anti-infectious or anticancer therapy can be advantageously combined with immunotherapy. The administration of the reprogrammed CD8+ T cells and at least one other therapy may be concomitant or sequential. For sequential administration, the reprogrammed CD8+ T cells may be administered before or after the other therapy. For example, anticancer chemotherapy or radiotherapy may be before adoptive T cell therapy with the reprogrammed CD8+ T cells.

Anticancer therapy includes chemotherapy, targeted therapy and/or radiotherapy. Immunotherapy includes therapies with cytokines, such as IL-15 therapy, or cytokine agonists; therapy with monoclonal antibodies such as targeting inhibitory immune checkpoint; co-stimulatory antibodies. In particular, checkpoint inhibitors include antibodies anti-PD1, anti-PD-L1, anti-CTLA-4, anti-TIM-3, anti-LAG3, anti-NKG2A. Co-stimulatory antibodies deliver positive signals through immune-regulatory receptors including but not limited to ICOS, CD137, CD27, OX-40 and GITR.

Anti-infectious therapy includes the use of the known antibacterial, antiviral, antiparasitic, antifungal agents currently used for treating infectious diseases. In some embodiments, the anti-infectious therapy is antiviral therapy, preferably antiretroviral therapy, in particular combined antiretroviral therapy (cART) or highly active antiretroviral therapy (HAART) as well-known in the art. Usually cART comprising a three-drug regimen containing a nonnucleoside reverse transcriptase inhibitor (NNRTI), a protease inhibitor (PI), or an integrase strand transfer inhibitor (INSTI), plus two nucleoside/tide reverse transcriptase inhibitors (NRTIs).

The invention encompasses the in vitro use of a GSK3 inhibitor for reprogramming CD8+ T cells according to the present disclosure. The invention encompasses also a composition comprising reprogrammed CD8+ T cells produced according to the method of the present disclose for use as a medicament, in particular for the treatment of an infectious disease or cancer in a patient in need thereof.

### Screening Method

Another aspect of the invention relates to a screening method for inducers of reprogramming of CD8+ T cells comprising: (a) administering a GSK3 inhibitor to a population of CD8+ T cells in vitro and (b) measuring the level of expression of TCF-1, CCR7, CD27, CD127, and/or the level of activation of mTORC in the population of CD8+ T cells. The method is useful for screening GSK3 inhibitors suitable for CD8+ T cell reprogramming aiming to induce CD8+ T cells having enhanced stemness and functional properties. In some embodiments, the level of expression of TCF-1, CCR7, CD27 and/or CD127 is increased in the population of CD8+ T cells by the GSK3 inhibitor. In some embodiments, the level of activation of mTORC1 is decreased and/or the level of activation of mTORC2 is increased in the population of CD8+ T cells by the GSK3 inhibitor. The level of expression of TCF-1, CCR7, CD27 and/or CD127 and the level of activation of mTORC may be determined according to the routine techniques, well-known of the person skilled in the art as disclosed herein.

Another aspect of the invention relates to a screening method for inducers of reprogramming of CD8+ T cells comprising: (a) administering a GSK3 inhibitor to a population of CD8+ T cells in vitro and (b) measuring: (i) the level of expression of: CD127; the effector transcription factor T-bet; activation marker(s), in particular CD38 and HLA such as HLA-DR; and/or inhibitory receptor(s), in particular PD-1 and LAG-3; (ii) the level of activation of mTORC (mTORC1, mTORC2) and/or (iii) the polyfunctionality in the population of CD8+ T cells. The method is useful for screening GSK3 inhibitors suitable for CD8+ T cell reprogramming aiming to modulate (enhance or repress) effector CD8+ T cells. The level of expression of CD127, T-bet, HLA-DR, CD38, PD-1, and/or LAG-3; the level of activation of mTORC and the polyfunctionality may be determined according to the routine techniques, well-known of the person skilled in the art as disclosed herein.

In some embodiments, the frequency of CD127− T-bet+; LAG-3+PD-1+; and/or HLA-DR+CD38+ cells is increased in the population of CD8+ T cells by the GSK3 inhibitor. In some embodiments, the frequency of CD127− T-bet+; LAG-3+PD-1+; and/or HLA-DR+CD38+ cells is decreased in the population of CD8+ T cells by the GSK3 inhibitor. In some embodiments, the polyfunctionality of the CD8+ T cells in response to TCR stimulation is increased in the population of CD8+ T cells by the GSK3 inhibitor, characterized by a higher frequency of TNF-α+ and IFN-γ+CD8+ T cells. In some embodiments, the polyfunctionality of the CD8+ T cells in response to TCR stimulation is decreased in the population of CD8+ T cells by the GSK3 inhibitor, characterized by a lower frequency of TNF-α+ and IFN-γ+CD8+ T cells. In some embodiments, the level of activation of mTORC1 and mTORC2 in response to TCR stimulation is increased in the population of CD8+ T cells by the GSK3 inhibitor, characterized by a higher frequency of pS6⁺pAKT⁺CD8+ T cells. In some embodiments, the level of activation of mTORC1 and mTORC2 in response to TCR stimulation is decreased in the population of CD8+ T cells by the GSK3 inhibitor, characterized by a lower frequency of pS6⁺pAKT⁺CD8+ T cells.

For example, effector CD8+ T cells are repressed with said GSK3 inhibitor when the frequency of CD127− T-bet+; LAG-3+PD-1+; HLA-DR+CD38+; Effector memory (TEM) and/or Terminal effector (TTE) cells is lower in CD8+ T cells treated with the GSK3 inhibitor than in non-treated cells (control). For example, effector CD8+ T cells are repressed with said GSK3 inhibitor when the reprogrammed CD8+ T cells have a reduced polyfunctionality and/or a low activation of mTORC1 and mTORC2 pathways in response to TCR stimulation.

For example, effector CD8+ T cells are enhanced with said GSK3 inhibitor when the frequency of CD127− T-bet+; LAG-3+PD-1+; HLA-DR+CD38+; Effector memory (TEM) and/or Terminal effector (TTE) cells is higher in CD8+ T cells treated with the GSK3 inhibitor than in non-treated cells (control). For example, effector CD8+ T cells are enhanced with said GSK3 inhibitor when the reprogrammed CD8+ T cells have an enhanced polyfunctionality and/or a high activation of mTORC1 and mTORC2 pathways in response to TCR stimulation.

In some embodiments, the screening method further or alternatively comprises measuring the survival, polyfunctionality, proliferation capacity, metabolic plasticity, response to antigen, response to γ-chain cytokines, response to immune check-point modulators, cytotoxic effect, and/or less mTORC1-dependency of the population of CD8+ T cells. These properties may be measured according to the routine techniques, well-known of the person skilled in the art as disclosed herein. The method may use CD8+ T cells from patients or appropriate model of the disease, in particular cancer or infectious disease.

The GSK3 inhibitor can be a chemical such as small organic molecule; small RNA molecule such as siRNA, miRNA, ribozyme, modified or unmodified; epigenome editing enzyme complex such as derived from CRISPR/Cas, TALENs, or Zinc-Finger nucleases; peptide, antibody, aptamer or other antagonist. In some embodiments, the GSK3 inhibitor is selected from the group consisting of: Anilinomaleimides, ions, Pyrroloazepines, Flavones, Benzazepinones, Bis-Indoles, in particular indirubins, Pyrrolopyrazines, Aminopyrimidines, Oxindoles, Thiazoles, Bisindolylmaleimides, Phenylaminopyrimidines, Pyrrolopyrimidines, Pyrazines, Thiadiazolidinones, Oxazole-carboxamides, and combinations thereof. In some preferred embodiments, the GSK3 inhibitor is selected from the group consisting of: SB-415286, Lithium carbonate, 10Z-Hymenialdisine, Flavopiridol hydrochloride hydrate, Alsterpaullone, 6-bromoindirubin-3′-oxime and 6-bromoindirubin-3′-acetoxime, Aloisine A, CHIR 98014, SU9516, AR-A014418, Staurosporine, GF 109203X, CGP 60474, TWS119, AZD2858, NP031112, SB-216763, PF-04802367, functional analogs or derivatives thereof, and combinations thereof.

In some embodiments, the screening method further comprises isolating, stimulating, expanding, engineering, and/or activating the CD8+ T cells according to the present disclosure. In some embodiments, the screening method further comprises administering the CD8+ T cells to the subject or patient according to the present disclosure.

“a”, “an”, and “the” include plural referents, unless the context clearly indicates otherwise. As such, the term “a” (or “an”), “one or more” or “at least one” can be used interchangeably herein; unless specified otherwise, “or” means “and/or”.

The practice of the present invention will employ, unless otherwise indicated, conventional techniques which are within the skill of the art. Such techniques are explained fully in the literature.

The invention will now be exemplified with the following examples, which are not limitative.

### EXAMPLES

### Materials and Methods

### Participants

Samples from people without HIV were obtained from the Etablissement Français du Sang in the context of a collaboration agreement with Institut Pasteur. HIV-1 non-controller participants (on antiretroviral treatment for at least 2 years and undetectable HIV-1 RNA) were recruited in the context of the ANRS EP36 XIII mTOR study or ANRS CO6 PRIMO cohort. Clinical data for participants included are summarized in Table 1. All participants gave their informed consent, and the study was approved by the ethics committee (Comité de Protection des Personnes) of Île-de-France XI. All analyses were done on cryopreserved PBMCs isolated from blood samples.

TABLE 1
Clinical characteristics of people with HIV included in the study

| | Age at | | CD4⁺ T | Viral load |
|---|---|---|---|---|
| | inclusion | | cell count | (RNA |
| Participant no | (years) | Gender | (cells/μL) | copies/μL) |
| 1 | 56 | Male | 962 | <40 |
| 2 | 57 | Male | 678 | <40 |
| 3 | 61 | Male | 919 | <40 |
| 4 | 47 | Female | 727 | <40 |
| 5 | 56 | Male | 1030 | <40 |
| 6 | 55 | Male | 415 | <40 |
| 7 | 54 | Male | 525 | <40 |
| 8 | 51 | Female | 957 | <40 |
| 9 | 54 | Female | 678 | <40 |
| 10 | 50 | Male | 592 | <20 |
| 11 | 64 | Male | 942 | <20 |
| 12 | 45 | Male | 791 | <20 |
| 13 | 54 | Male | 390 | <20 |
| 14 | 72 | Male | 1032 | <20 |
| 15 | 57 | Male | 501 | <20 |
| 16 | 42 | Female | 1282 | <20 |
| 17 | 29 | Male | 550 | <20 |
| 18 | 37 | Male | 519 | <20 |
| 19 | 36 | Male | 989 | <20 |

### In Vitro Reprogramming of CD8⁺ T Cells

Frozen PBMC were thawed and rested overnight at 37° C., 5% CO₂, in RPMI 1640 GlutaMAX medium supplemented with 10% fetal calf serum and penicillin/streptomycin (complete medium). For reprogramming of CD8⁺ T cells followed by polyclonal stimulation, cells were isolated by negative magnetic bead sorting (Stemcell Technologies). For reprogramming of CD8⁺ T cells followed by antigen-specific stimulation, PBMC and magnetically separated CD8⁺ T cells and non-CD8⁺ T cells were used (REAlease® CD8 MicroBead Kit; Miltenyi Biotech). Then, CD8⁺ T cells were treated with a GSK3 inhibitor (6-Bromoindirubin-3′-oxime; BIO; Sigma-Aldrich), at 3 μM, or an equivalent concentration of dimethyl sulfoxide (DMSO) vehicle control, in complete medium, for 12 hs at 37° C., 5% CO₂. Non-CD8 cells were left resting during this time in complete medium. CD8⁺ T cells were washed with complete medium and stimulated with the peptide pool in presence of non-CD8 T cells. To confirm the effects of BIO, another GSK3 inhibitor, TWS119 (Sigma-Aldrich), was used under similar conditions (at 3 μM, 12 hs incubation); other GSK inhibitors (Table 2) were used under same incubation time (12 hs) and at the concentration indicated in Table 2.

### Polyclonal, Antigen-Specific, and Cytokine Stimulation of CD8⁺ T Cells

After reprogramming, CD8⁺ T cells were polyclonally stimulated with plate-bound anti-CD3 and CD28 antibodies (both at 1 μg/mL; clones OKT3 and CD28.2, respectively; both from Thermo Fisher), in the presence or absence of human recombinant ICAM-1/CD54 Fc Chimera Protein (at 50 μg/mL; R&D) and cultured for 48 hs at 37° C., 5% CO₂, accordingly. Antigen-specific stimulation was performed by mixing CD8⁺ and non-CD8 cells (maintaining the original cell ratio) and incubating with overlapping peptide pools encompassing HIV-1 consensus subtype B Gag or HCMV pp65 (both at 2 μg/ml; obtained through the National Institute of Health (NIH) AIDS Reagent Program, Division of AIDS, National Institute of Allergy and Infectious Diseases, NIH, catalog numbers 12425 and 11549). In some experiments, cells were incubated with an anti-PD-1 blocking antibody (clone J116, Thermo Fisher Scientific; at 10 μg/mL). For subsequent intracellular cytokine analysis, cells were incubated for 6 hs in the presence of anti-CD28+CD49d antibodies (clones L293 and L25, respectively; both at 1 μg/mL; BD Biosciences), an anti-CD107a FITC antibody (BD Biosciences), plus brefeldin A (at 10 μg/mL; Sigma-Aldrich) and monensin (at 1 μg/mL; BD Biosciences), the two latter added 30 min after the start of all incubations. In some experiments, cells were culture in RPMI medium without glucose (MP Biomedicals). CD8⁺ T cell proliferation was evaluated by CFSE dilution (at 1 μM, Thermo Fisher Scientific). In additional experiments, reprogrammed CD8⁺ T cells were stimulated with IL-7 or IL-15 (both at 10 ng/mL; R&D) and cultured for 6 days. In all the cases, cells were cultured at a density of 2×10⁶ cells/mL.

### Flow Cytometry Analysis

After cultures, cells were stained with the LIVE/DEAD Fixable Aqua Dead Cell Stain kit (Thermo Fisher Scientific), with anti-CD3 Alexa Fluor 700 or APC efluor 780 and anti-CD8 PE Texas Red antibodies, accordingly. For phenotype analyses, cells were additionally stained with anti-CCR7 PE Cy7, anti-CD45RA APC H7, anti-CD27 PerCP Cy5.5, anti-CD95 APC, or anti-CD127 Alexa Fluor 488 antibodies, incubated for 15 minutes at room temperature. In some experiments, cells were stained with APC-conjugated dextramers (Immudex), incubated for 15 minutes at room temperature. Additional surface staining panels included anti-HLA-DR Superbright 780, anti-CD38 Superbright 600, anti-PD-1 BV421, anti-LAG-3 APC efluor 780, anti-TIM3 PE Cy7, and anti-TIGIT BV786 antibodies, or anti-CD122 PE and anti-CD215 FITC antibodies. The BD Transcription Factor Buffer Set kit (BD Biosciences) was used for cell fixation and permeabilization. Intracellular staining panels for detection of transcription factors included anti-TCF-1 PE, anti-T-bet V450, anti-Eomes APC, anti-TOX efluor 660, anti-BLIMPI CF594, anti-BCL6 Alexa Fluor 488, or anti-Nur77 Alexa Fluor 647, accordingly. Intracellular cytokine staining panels included anti-IFN-γ PE Cy7 or V450, anti-IL-2 APC-R700, anti-TNF-αPerCP Cy5.5, or anti-granzyme B Alexa Fluor 647 antibodies, accordingly. In some experiments, cells were fixed and permeabilized with Phosflow fix/perm buffers (BD Biosciences) and cells were stained with anti-pS6 Ser235/236 Pacific blue and anti-pAKT Ser473 FITC antibodies (Cell Signaling). Cell acquisition was performed using LSR II or ARIA III flow cytometers (both from BD Biosciences), and data were analyzed with FlowJo v.10 software (BD Biosciences).

### Measurement of Metabolite Uptake

CD8⁺ T cells were split into four parts and put into contact with 2-NBDG (2-(N-(7-Nitrobenz-2-oxa-1,3-diazol-4-yl)Amino)-2-Deoxyglucose) (150 μM, 30 min) for glucose uptake measurement, BODIPY 500/510 C₁, C₁₂ (4,4-Difluoro-5-Methyl-4-Bora-3a,4a-Diaza-s-Indacene-3-Dodecanoic acid) (5 μM, 5 min) for fatty acid uptake measurement, MitoTracker Green FM (100 nM, 45 min) for mitochondrial mass measurement, and CellROX Deep Red (5 μM, 30 min) for measurement of oxygen reactive species (all from Thermo Fisher Scientific). After these incubations, cells were stained with the LIVE/DEAD Fixable Aqua Dead Cell Stain kit, as well as with phenotype antibodies, as described above.

### Gene Expression Analysis in Sorted Memory Cells

Purified CD8⁺ T cells were stained with the LIVEDEAD Fixable Aqua Dead Cell Stain Kit (Thermo Fisher Scientific) and the following antibodies: anti-CD3 Alexa Fluor 700, anti-CD8 APC Cy7, anti-CCR7 PE Cy7, anti-CD45RA BV421, and anti-CD27 PE (all from BD Biosciences). Viable central memory, transitional memory, effector memory, and terminal effector CD8⁺ T cells were bulk sorted (BD FACS ARIA III, BD Biosciences), left rested for at least 6 hs in complete medium before treatment with the GSK3 inhibitor or vehicle control. Then, cells were washed and stimulated with plate-bound anti-CD3/CD28 (1 μg/mL) for 48 hs. After culture, cells were counted, and a total of 2,500 cells per condition were put in 96-well plates containing the VILO Reaction Mix, SUPERase-In, and NP40 (all from Thermo Fisher Scientific). Plates were snap-frozen and stored at −80° C. Analysis of gene expression was performed as previously described (8), using Delta Gene primers, 96.96 Dynamic Array chips, and a Biomark instrument for microfluidics-based qPCR (Fluidigm). Linear derivative mode baseline correction was applied. Data were normalized using GAPDH as a housekeeping gene and using the delta Ct method. Gene expression values are plotted as 2^{-deltaCt}.

### Statistical Analyses

GraphPad Prism software version 9.0 was used for statistical analysis. Data are presented as medians and ranges. The Wilcoxon test was used for the comparison of two paired data. The Friedman test, and the Dunn's multiple comparisons test, were used for the comparison of three or more paired groups. All P values less than 0.05 were considered as significant. To determine differentially expressed genes for each CD8⁺ T cell memory population, a mixed effect model was defined including treatment (vehicle control versus GSK3 inhibitor), condition (unstimulated or anti-CD3/CD28 stimulated), and their interaction as fixed effects, as well as considered patient and technical replicates as a random effect. The parameters have been estimated using the lme4 package (v1.1-23).

### Example 1: CD8⁺ T Cell Reprogramming Towards a Stem-Like Memory Profile

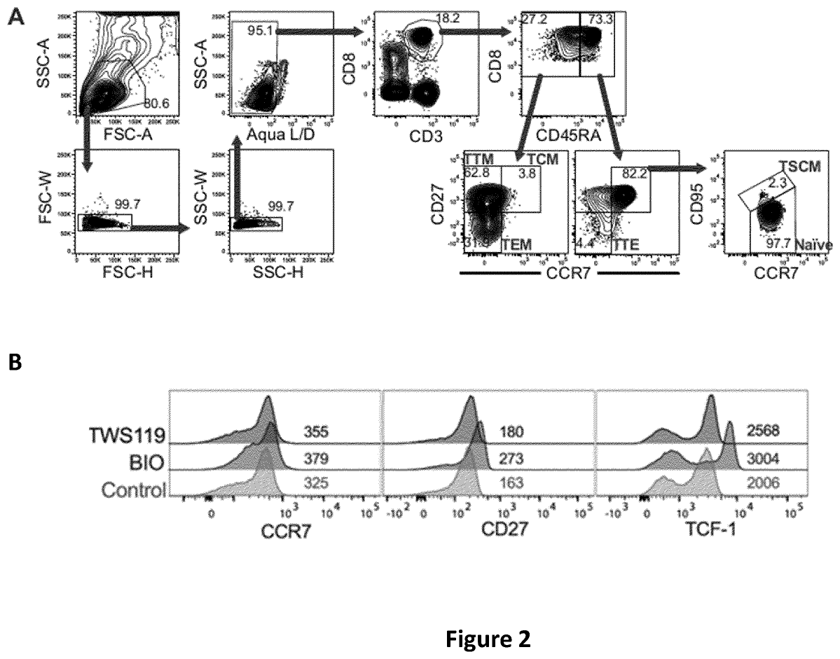

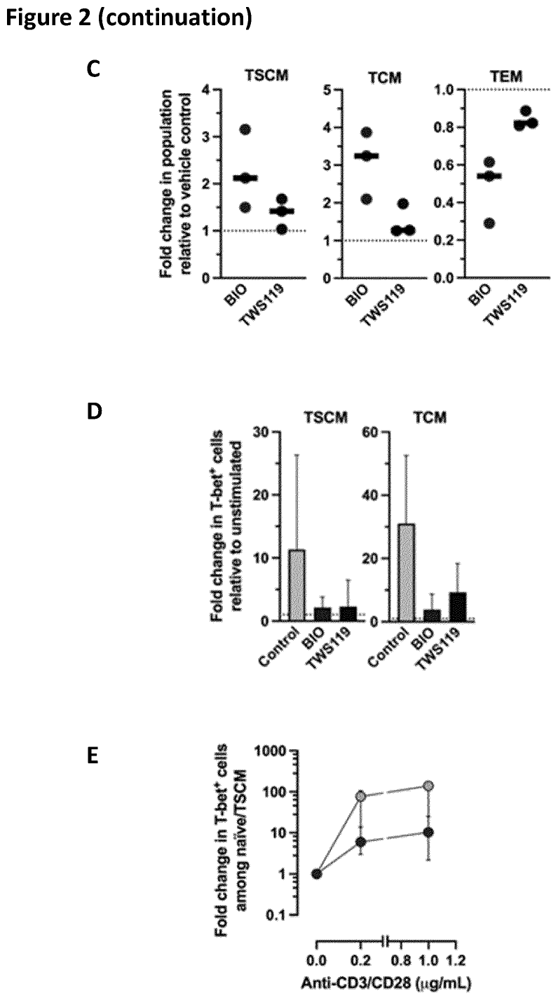

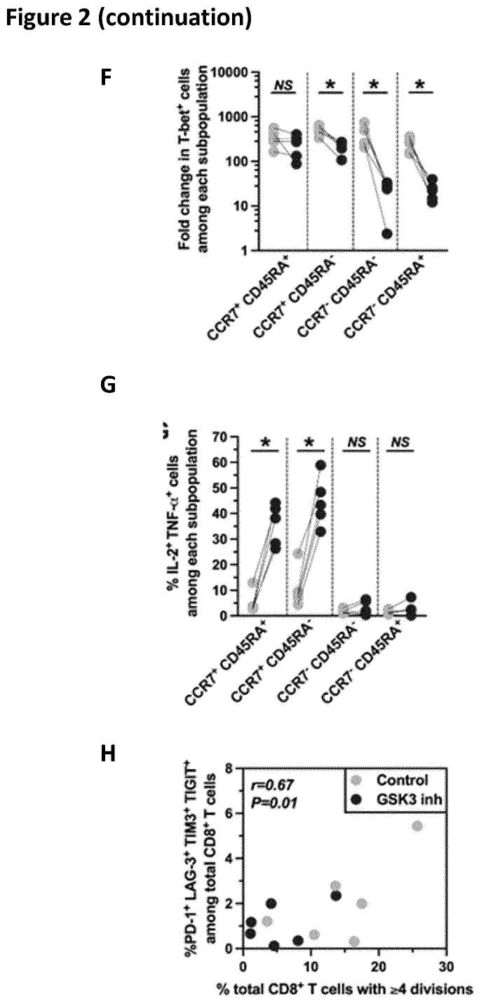

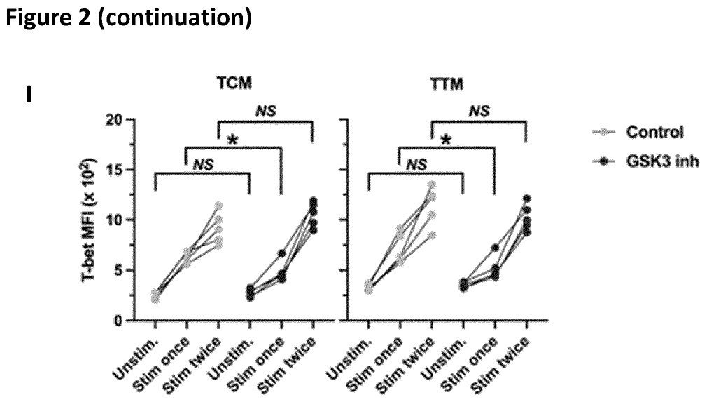

To test the hypothesis that CD8⁺ T cell reprogramming can invigorate stem-like properties, the inventors used the glycogen synthase kinase-3 (GSK3) inhibitor 6-Bromoindirubin-3′-oxime (BIO), which modulates pathways involved in the generation and maintenance of stem-like CD8⁺ T cells, such as Wnt/β-catenin and mTORC2 (12), that them and others have found to be upregulated in CD8⁺ T cells from natural controllers (7,8,16). The effect of BIO-mediated reprogramming on bulk CD8⁺ T cells from people without HIV was first characterized. Treatment with BIO (in the absence of further stimulation), was accompanied by upregulation of surface expression of CCR7 and CD27 (FIG. 1A). Accordingly, it was observed an enrichment of CD8⁺ T cells with a less-differentiated stem cell memory (TSCM) and central memory (TCM) phenotype, and a decrease of more-differentiated effector memory (TEM), and terminal effector (TTE) cells (FIGS. 1A-C and FIG. 2A. The effect of the GSK3 inhibitor was confirmed by the enhanced expression of TCF-1 (FIG. 1D). To confirm that these changes were related to the regulation of GSK3 activity, the impact of TWS119, another GSK3 inhibitor previously used to promote stem-like memory CD8⁺ T cells (21) was also assessed. The treatment with both GSK3 inhibitors under parallel experimental conditions induced CCR7, CD27, and TCF-1 (FIG. 2B), increase of TSCM and TCM cells, and decrease of TEM cells (FIG. 2C). However, BIO consistently showed a greater effect than TWS119 (FIGS. 2B and C) and was used throughout the study. These results confirm that GSK3 inhibition, hereinafter referred to as CD8⁺ T cell reprogramming, promotes a memory-like phenotype in CD8⁺ T cells.

It was wondered whether reprogrammed CD8⁺ T cells maintained their memory-like profile upon activation. Following anti-CD3/CD28 stimulation, reprogrammed CD8⁺ T cells, when compared to cells in the control condition, exhibited lower expression of the activation markers HLA-DR and CD38, the inhibitory receptors PD-1, LAG-3, and TIM-3, and the transcription factors T-bet and BLIMP-1, which are associated with an effector CD8⁺ T cell profile (FIG. 1E and FIG. 2D) (22). On the contrary, reprogrammed CD8⁺ T cells maintained higher levels of the transcription factors BCL-6, TCF-1, and TOX, associated with a memory profile and restriction of terminal effector differentiation (23-25), as well as CD127, critical for CD8⁺ T cell survival (FIG. 1E) (26). Consistent with the higher expression of CD127 and restriction of T-bet, reprogrammed CD8⁺ T cells showed lower levels of activation-induced cell death than cells in the control condition (FIG. 1F). The effects of CD8⁺ T cell reprogramming were also observed in the setting of a lower and higher TCR-mediated stimulation with anti-CD3/CD28±ICAM-1 (27) since reprogrammed CD8⁺ T cells maintained lower frequencies of T-bet⁺ cells than non-reprogrammed cells (FIGS. 2E and F).

### Example 2: Reprogrammed CD8⁺ T Cells Show Enhanced Polyfunctionality

As reprogramming of CD8⁺ T cells decreased the proportion of effector cells and restricted cell activation, it was wondered whether reprogrammed CD8⁺ T cells could acquire functional capacity. Consistent with the restriction of an effector-like transcriptional program, reprogrammed total CD8⁺ T cells had lower expression of granzyme B and IL-2 than non-reprogrammed cells although there was no change in IFN-γ production (FIG. 1G). In contrast, there was a marked increase in the capacity of reprogrammed CD8⁺ T cells to produce TNF-α and overall increased polyfunctionality (FIGS. 1G and H). Higher polyfunctionality was also observed after stimulation with anti-CD3/CD28+ICAM-1 (FIG. 2G). The impact of CD8⁺ T cell reprogramming on the proliferative capacity and effector maturation upon sequential polyclonal re-stimulation was also assessed. Interestingly, reprogramming did not affect CD8⁺ T cell capacity to execute 1-3 division cycles but prevented extensive proliferation (≥4 cell divisions) and maintained a higher proportion of quiescent cells (FIG. 1I). This pattern of regulated proliferation of reprogrammed cells correlated with their lower coexpression of PD-1, LAG-3, TIM3, and TIGIT (FIG. 2H). Notably, reprogramming did not irreversibly arrest effector maturation of less-differentiated TCM and transitional memory (TTM) cells since these subsets could readily upregulate T-bet under the re-stimulation setting (FIG. 2H).

Moreover, it was evaluated if CD8⁺ T cell reprogramming affects their function at the antigen-specific level. To this end, cells from people without HIV were stimulated with human cytomegalovirus (HCMV) pp65 peptides and the phenotype and function of antigen-specific cells was evaluated. No significant differences were observed in the total frequencies of CD8⁺ T cells responding to HCMV pp65 peptides between reprogrammed versus non-reprogrammed cells (FIG. 1J), indicating that the pharmacological manipulation of the cells does not affect the TCR-mediated response. However, an increase of HCMV pp65-specific CD8⁺ T cells with a TCM phenotype, and a decrease of TTM and TEM, were readily observed after reprogramming (FIG. 1K). Importantly, CD8⁺ T cell reprogramming enhanced TNF-α expression (FIG. 1L) and did not affect the effector-like functions of HCMV pp65-specific CD8⁺ T cells, including IFN-γ and CD107a expression (FIG. 1L), neither their expansion capacity after 6 days (FIG. 1M). Collectively, these results indicate that CD8⁺ T cell reprogramming induces TNF-α production and polyfunctionality, without impairing the effector-like functions and expansion of antigen-specific cells.

### Example 3: Reprogramming Modifies the Functional Properties and Transcriptional Signature of all CD8⁺ T Cell Memory Subsets

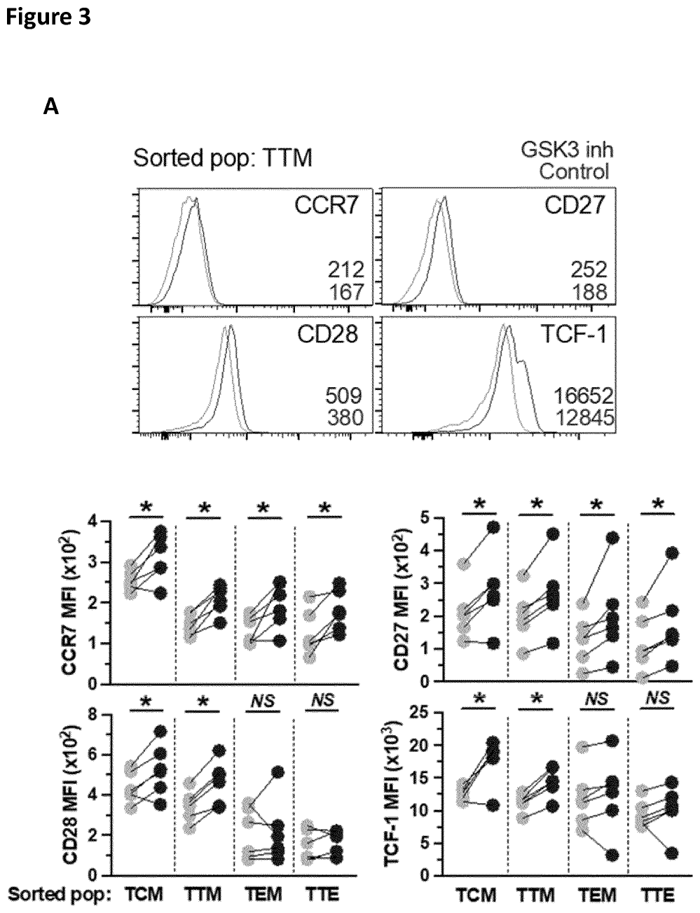

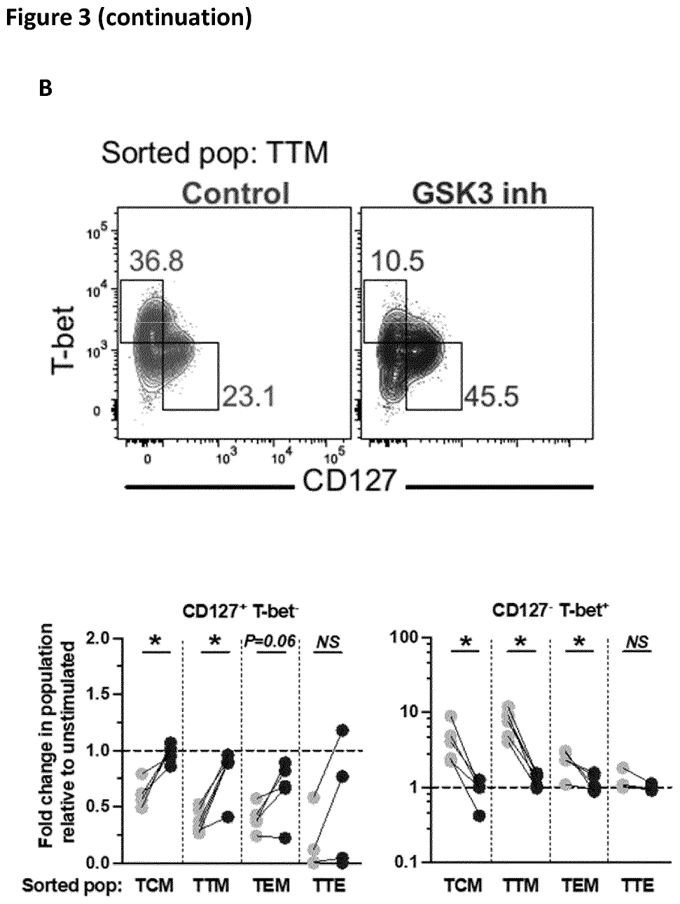

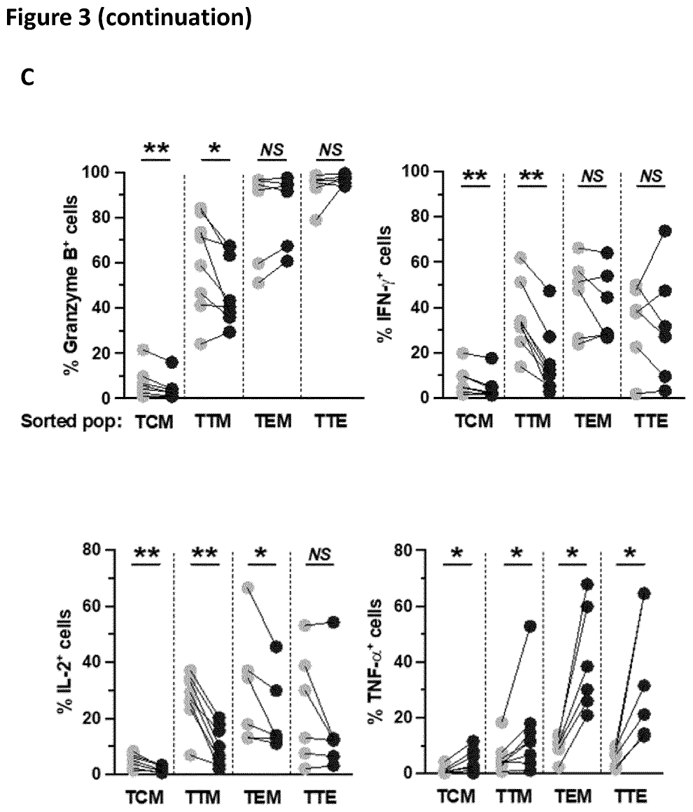

It was asked if the changes induced by cell reprogramming were related to phenotype conversion between CD8⁺ T cell subpopulations or were intrinsic to each subset. To solve this issue, TCM, TTM, TEM, and TTE CD8⁺ T cells were isolated and treated with the GSK3 inhibitor, followed by resting or stimulation with anti-CD3/CD28. In basal conditions, cell reprogramming caused the upregulation of CCR7 and CD27 in all cell subsets (FIG. 3A). Thus, the enrichment in less-differentiated cells observed after CD8⁺ T cell reprogramming (FIG. 1A-C) is most likely the result of the conversion of CD27⁻ and CCR7⁻ cells into CD27⁺ and CCR7⁺ subsets. In addition, reprogramming further enhanced the expression of CD28 and TCF-1 in TCM and TTM cells (FIG. 3A), while preventing the loss of CD127 and limiting the upregulation of T-bet upon stimulation of CD8⁺ T cell memory subsets (FIG. 3B). Notably, reprogramming of TCM and TTM cells restrained the expression of granzyme B, IFN-γ, and IL-2, but TEM and TTE cells maintained these effector properties (FIG. 3C). As was observed with bulk CD8⁺ T cells, all reprogrammed CD8⁺ T cell subsets exhibited an increased TNF-α production (FIG. 3C).

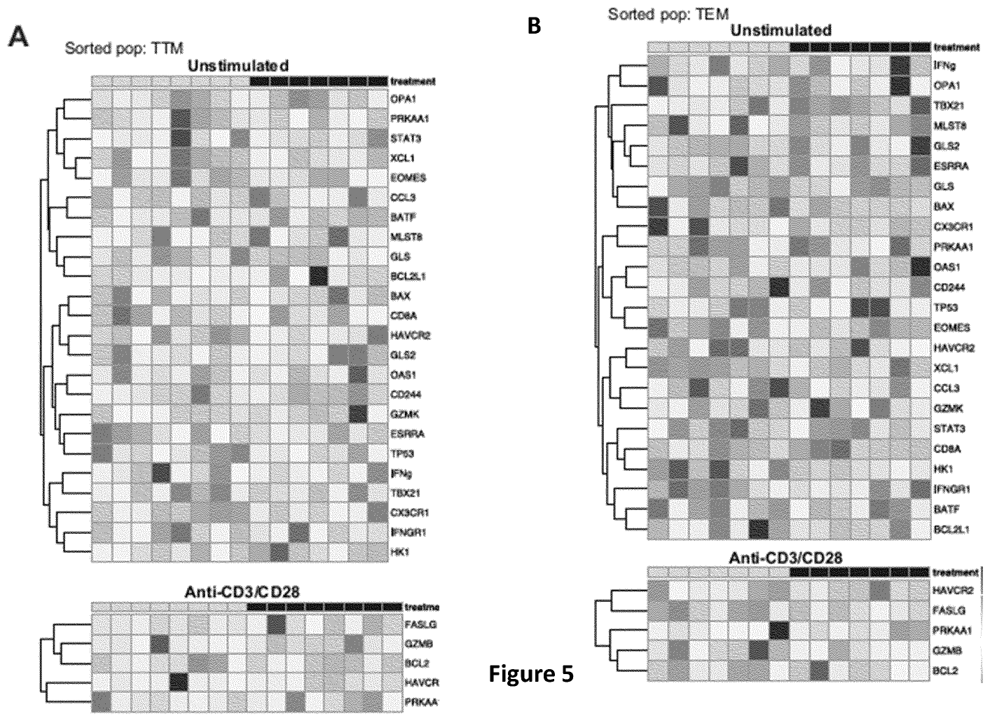

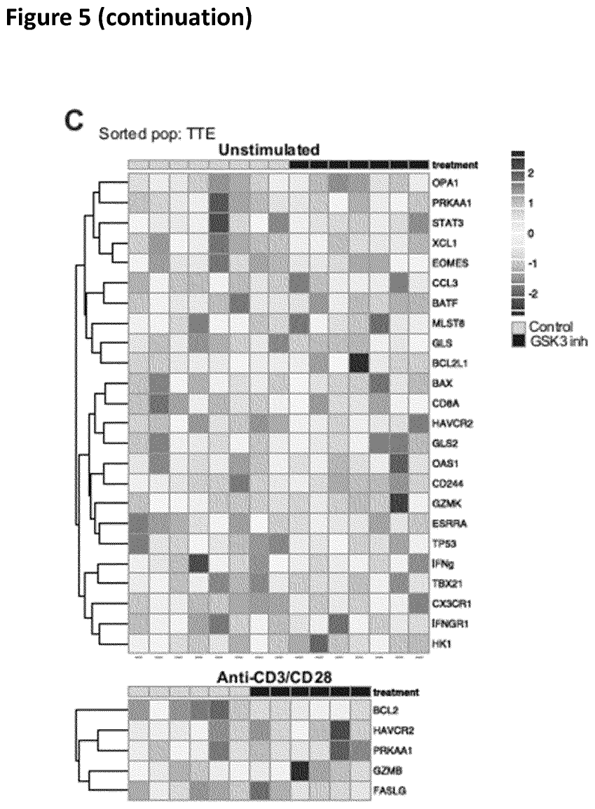

To better characterize the changes induced in reprogrammed CD8⁺ T cells, a gene expression analysis of sorted memory cells was performed, analyzing 96 genes associated with CD8⁺ T cell function, differentiation, metabolism, and survival. A principal component analysis (PCA) focused on TCM cells showed that reprogrammed and non-reprogrammed cells had a distinct gene expression profile (FIG. 4A). In basal conditions, compared with non-reprogrammed cells, reprogrammed TCM cells exhibited lower levels of IFNG, BATF, IFNGR1, and GZMK genes, which are associated with an effector lineage (28,29), as well as CD8A, CD244, and HAVCR2 (which codifies for TIM3), which are associated with CD8⁺ T cell activation and exhaustion (30) (FIG. 4B). Reprogrammed CD8⁺ T cells exhibited in contrast a gene profile suggestive of metabolic quiescence (31), with lower levels of key metabolic regulators active during T cell activation, including MLST8 (which codifies for MTOR Associated Protein, LST8 Homolog; required for mTORC pathway activation (32)), ESRRA (which codifies for Estrogen-related receptor ametabolic regulator of effector T cells (33)), GSL and GSL2 (which codify for glutaminase 1 and 2, respectively (34)), and PRKAA](which codifies for AMP-activated protein kinase; important regulator of cell catabolic pathways (35)) (FIG. 4B). Importantly, after anti-CD3/CD28 stimulation, reprogrammed TCM cells had higher levels of the antiapoptotic gene BCL2 than non-reprogrammed cells, and lower levels of effector-associated genes FASLG, HAVCR2, and GZMB, as well as PRKAA1 (FIG. 4B). The gene expression profile in reprogrammed TTM, TEM, and TTE was also modified by reprogramming (FIG. 5), and, among others, higher expression of BCL2 and the mTORC2-related gene CDC42 was observed in these memory subsets (FIG. 4B and FIG. 5), explaining in part the improved survival capacity of reprogrammed CD8⁺ T cells.

### Example 4: The Stem-Like Profile and Polyfunctionality of Reprogrammed CD8⁺ T Cells is Associated with the Downregulation of Anabolic Metabolism and the mTORC1 Pathway

Considering that regulation of cell metabolism and mTORC pathways is a characteristic of memory-like CD8⁺ T cells (36), as previously reported for cells from HICs (8,37), and that CD8⁺ T cell reprogramming readily modulated metabolism-related genes functional assays were performed to evaluate the metabolic profile of reprogrammed cells. After anti-CD3/CD28 stimulation, non-reprogrammed cells increased their glucose (2-NBDG) and lipid (BODIPY) uptake, augmented the mitochondrial mass, and the production of reactive oxygen species (ROS) (FIG. 4C), a profile characteristic of recently activated T cells (31). However, reprogrammed CD8⁺ T cells showed lower levels of each of these metabolic parameters (FIG. 4D), consistent with reduced anabolic metabolism. In addition, the expression of Nur77, a nuclear receptor recently shown to regulate the metabolic switch during T cell activation (38) was evaluated. According to the restricted metabolic activity that was observed in reprogrammed CD8⁺ T cells, they exhibited higher levels of Nur77 after TCR-mediated stimulation in comparison with non-reprogrammed cells (FIG. 4E). Moreover, the Nur77⁺ subset exhibited greater polyfunctionality (FIG. 4F). The higher expression of Nur77 appeared to be directly related to the expression of TCF-1 since TCF-1V cells showed higher levels of Nur77 compared to TCF-1⁻ cells, both in non-reprogrammed and reprogrammed cells (FIG. 4G). These data are in agreement with a recent study showing higher levels of Nur77 in CD62L^{hi} TCF-1^{hi} memory precursor cells (39) and support the role of this nuclear receptor in the metabolic regulation of stem-like memory cells.

It was next focused on the mTORC pathways and their activation was evaluated by analyzing the phosphorylation of ribosomal S6 (pS6 Ser235/236) and AKT (pAKT Ser473) proteins, markers of activation of mTORC1 and mTORC2, respectively (40). Upon activation in control conditions, CD8⁺ T cells upregulated pS6 in parallel with pAKT (pS6⁺pAKT⁺ cells; FIG. 4H). Reprogrammed CD8⁺ T cells had less upregulation of pS6 (FIGS. 4H and I). Notably, reprogrammed CD8⁺ T cells had higher levels of pS6⁻ pAKT⁺ (FIGS. 4H and I), confirming that reprogrammed cells maintained a relative metabolic quiescence supported by preferential upregulation of the mTORC2 pathway. Of note, despite the restriction of the mTORC1 pathway, reprogrammed CD8⁺ T cells had higher frequencies of IFN-γ⁺TNF-α⁺ cells (FIG. 4J). In addition, when analyzing IFN-γ and IL-2 production among TNF-α-producing CD8⁺ T cells upon polyclonal stimulation, two major subpopulations were identified: IFN-γ⁺ IL-2⁺ and IFN-γ⁻ IL-2⁻, the latter corresponding to cells producing TNF-α only and being increased among reprogrammed cells (FIG. 6). In keeping with previous studies on cells from HICs (8), the single production of TNF− by reprogrammed cells was less dependent on mTORC1, since the proportion of pS6⁺ cells was lower in this subset relative to cells co-producing IFN-γ and IL-2 (FIG. 6). Altogether, these data indicate that the stem-like profile and polyfunctionality observed in reprogrammed CD8⁺ T cells are linked to the active regulation of anabolic metabolism, mTORC1 inhibition, and preferential engagement of mTORC2. These data also highlight the differential role of mTORC pathways on the regulation of CD8⁺ T cell effector functions.

### Example 5: Reprogramming of HIV-Specific CD8⁺ T Cells Promotes Polyfunctionality, Survival, and Expansion

These results on polyclonally-stimulated or HCMV-specific cells from people without HIV indicated that reprogramming improves, and does not impair, several functional capacities of CD8⁺ T cells. However, HIV-specific CD8⁺ T cells from non-controllers have a biased program characterized by exhaustion, low survival, and poor antiviral potential (8). Thus, it was explored if HIV-specific CD8⁺ T cells from non-controller individuals can also benefit from reprogramming and acquire properties found in HIV-specific CD8⁺ T cells associated with natural control of infection. First, the effect of reprogramming was evaluated on the phenotype of CD8⁺ T cells labeled with HLA-specific HIV dextramers. In line with the results on total CD8⁺ T cells from people without HIV, reprogramming of HIV dextramer⁺ cells resulted in the upregulation of CCR7, CD27, and TCF-1 (FIGS. 7A and B). Accordingly, a Uniform Manifold Approximation and Projection (UMAP) analysis focused on HIV dextramer⁺ cells revealed enrichment of populations with a less-differentiated phenotype after reprogramming (FIG. 7A), with an increase in the proportions of naïve/TSCM and TCM cells among reprogrammed HIV dextramer⁺ cells and a decrease of TEM and TTE cells (FIG. 7C).

Next, the functionality of HIV-specific CD8⁺ T cells, detected by the peptide-induced production of IFN-γ, IL-2, TNF-α, or by the expression of the degranulation marker CD107a was evaluated. Reprogrammed CD8⁺ T cells contained a significantly higher frequency of total HIV-specific responses (FIG. 7D), that were enriched on TSCM and TCM cells (FIGS. 7E and F). Instead, non-reprogrammed cells were mostly TEM and TTE cells (FIGS. 7E and F). Notably, higher production of TNF-α by reprogrammed cells was observed in terms of frequency (FIG. 7G) and intensity of expression (FIG. 7H) in HIV-specific CD8⁺ T cells, whereas there were no differences in the levels of CD107a, IFN-γ, granzyme B, and IL-2 (FIG. 7H). An overall increased polyfunctionality was observed in reprogrammed HIV-specific CD8⁺ T cells relative to non-reprogrammed cells (FIG. 7I). Of note, the survival capacity of reprogrammed HIV-specific cells was higher relative to non-reprogrammed cells (FIG. 7J), in agreement with the observation that reprogramming induced the upregulation of anti-apoptotic factors (FIG. 4B). This resulted in enhanced survival of HIV-specific cells over 6 days culture in the setting of sequential re-stimulation (FIG. 7K). Collectively, these results indicate that reprogramming of HIV-specific CD8⁺ T cells from non-controllers promotes a quantitatively and qualitatively superior response to antigen stimulation, reflected in higher functionality, survival, and expansion capacity, which are features observed in cells from natural HIV-1 controllers.

### Example 6: Metabolic Plasticity Restored in Reprogrammed HIV-Specific CD8⁺ T Cells

It was next assessed whether reprogramming could improve the marked dependency on mTORC1 and glycolysis that characterizes HIV-specific CD8⁺ T cells from HIV-1 non-controllers (8). Stimulation of non-reprogrammed cells from non-controllers with HIV-1 Gag peptides resulted in upregulation of pS6, both alone and together with pAKT (FIG. 8A). In contrast, reprogrammed HIV-specific CD8⁺ T cells had lower proportions of pS6+pAKT− cells (FIG. 8A). A significantly lower expression of pS6 was also observed on a per-cell basis in reprogrammed HIV-specific CD8⁺ T cells (FIG. 8B). In contrast, no significant differences were observed in the intensity of pAKT in reprogrammed cells (FIGS. 8A and B). These data indicate that reprogrammed HIV-specific CD8⁺ T cells had a diminished dependency on mTORC1 for supporting a strong antigen-induced response, while preserving mTORC2 activation to exert their functions. Additionally, glucose dependency of reprogrammed HIV-specific CD8⁺ T cells was evaluated in an assay where Gag peptide stimulation was performed in medium with versus without glucose (8). Glucose deprivation decreased the frequency of total HIV-specific cells at comparable levels in reprogrammed versus non-reprogrammed cells (FIGS. 8C and D). However, reprogrammed cells maintained higher production of TNF-α despite glucose deprivation (FIG. 8E). Altogether, these data indicate that reprogramming of HIV-specific cells from non-controllers promotes metabolic plasticity and decreases metabolic restrictions, favoring their strong effector function.

### Example 7: Reprogrammed CD8⁺ T Cells Respond Better to Homeostatic γ-Chain Cytokines

The long-term maintenance of memory T cells requires an optimal response to the γ-chain cytokines IL-7 and IL-15 for self-renewal (41). Thus, it was evaluated how reprogramming impacted the capacity of CD8⁺ T cells to respond to these homeostatic cytokines. It was shown that reprogrammed bulk CD8⁺ T cells were characterized by higher levels of CD127 (IL-7Rα chain; FIG. 1E). It was also found that reprogrammed CD8⁺ T cells had higher expression of CD122 (IL-2Rβ chain, component of the IL-15 receptor) and the IL-15Rα chain CD215 (FIGS. 9A and B), as well as a higher proportion of cells co-expressing the transcription factor Eomesodermin (Eomes) and CD122 (FIG. 10A). This may be related to previous observations showing that TCF-1 regulates the expression of Eomes, which in turn promotes CD122 (24).

The increased expression of IL-7 and IL-15 receptors in reprogrammed bulk CD8⁺ T cells from people without HIV was reflected in augmented proliferation in response to both cytokines when compared to non-reprogrammed cells (FIG. 10B). As expected, the proliferative potential was higher in naïve/TSCM and TCM cells than in more differentiated cells (FIG. 10B). Reprogramming also improved the proliferative response to IL-15 of HIV dextramer⁺ cells from HIV-1 non-controllers (FIGS. 10C and D). Finally, the maturation of the effector response induced by IL-15 in terms of upregulation of T-bet was comparable between reprogrammed and non-reprogrammed cells (FIG. 9C). Overall, these results show that CD8⁺ T cell reprogramming improves the response to homeostatic γ-chain cytokines in cells with otherwise diminished capacity.

Next, the ex vivo response of reprogrammed HIV-specific CD8⁺ T cells to PD-1 blockade, which is currently being evaluated as immune checkpoint therapy in HIV-1 (NCT03787095) was evaluated. To this end, the proliferation of CD8⁺ T cells from non-controller individuals was evaluated after stimulation with Gag peptides in the presence or absence of an anti-PD1 antibody. Gag peptides stimulation alone induced proliferation of non-reprogrammed CD8⁺ T cells, but PD-1 blockade did not further improve cell expansion (FIG. 11A). In contrast, reprogrammed cells consistently had a better response to PD-1 blockade, with a significant increase in the frequency of proliferating HIV-specific cells relative to Gag peptides alone stimulation and compared to non-reprogrammed cells (FIG. 11A). Moreover, reprogrammed cells consistently increased the expression of TNF-α in response to PD-1 blockade, an effect that was not observed in non-reprogrammed cells (FIG. 11B). Thus, reprogrammed HIV-specific CD8⁺ T cells are more suited to respond to immune checkpoint blockade. This demonstrates that reprogramming also improved the response of HIV-specific CD8⁺ T cells to immunotherapies.

### Example 8: Screening Molecules for Reprogramming Human CD8⁺ T Cells

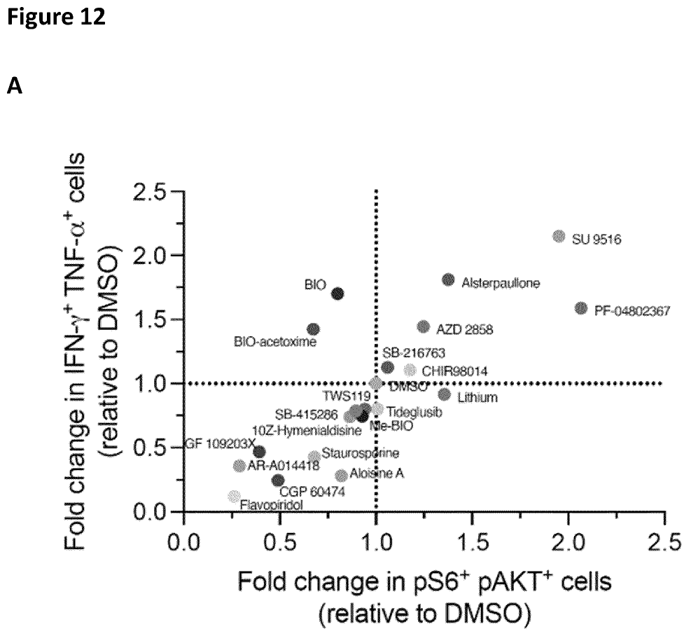

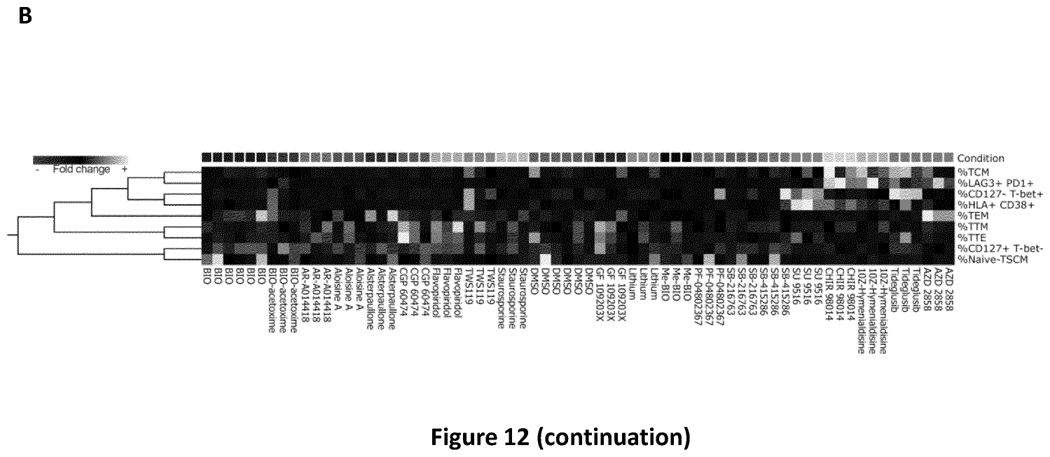

The inventors explored GSK3 inhibitors to expand the array of molecules that could be used for CD8⁺ T cell reprogramming. They implemented a screening approach that combined the evaluation of cytokine polyfunctionality of CD8⁺ T cells (IFN-γ+TNFα+) vs the activation of mTORC1 and mTORC2 pathways (pS6+pAKT+) upon treatment with the GSK3 inhibitors and subsequent polyclonal stimulation. This approach allowed the inventors to identify molecules that (i) promoted (high polyfunctionality+high activation of mTORC1 and mTORC2 pathways) or (ii) repressed (low polyfunctionality+low activation of mTORC1 and mTORC2 pathways) effector profiles upon stimulation of T cells, or (iii) promoted stem cell memory like profiles characterized by production of cytokines in the absence of the upregulation of both mTORC1 and mTORC2 pathways (high polyfunctionality+low frequency of cells with activated mTORC1 and mTORC2 pathways) (FIG. 12A). The inventors performed a screening of 20 GSK3 inhibitors from different chemical families, with varying affinity, specificity, and mechanism of inhibition of GSK3. Consistent with results presented in previous examples, treatment with BIO and the chemically related molecule BIO-acetoxime promoted IFN-γ⁺TNF-α⁺ cells relative to vehicle control, as well as a lower frequency of pS6⁺pAKT⁺ cells (FIG. 12A). However, this pattern was not observed for any other GSK3 inhibitor. Instead, some drugs such as SU 9516, alsterpaullone, and PF-04802367 increased the frequency of IFN-γ⁺TNF-α⁺ cells concomitant with activation of mTORC pathway (FIG. 12A), whereas some molecules such as AR-A014418 decreased both CD8⁺ T cell polyfunctionality and activation of mTORC pathway.

The inventors confirmed these patterns analyzing a larger range of phenotypic parameters. As expected, BIG and BI-acetoxime promoted a less-differentiated phenotype (T stem cell memory) and preserved a CD127⁺ T-bet⁻ phenotype, while restrained the acquisition of the effector transcription factor T-bet, the activation markers HLA-DR and CD38, and the inhibitory receptors PD-1 and LAG-3 (FIG. 12B). In contrast, other molecules such as Tideglusib (thiadiazolidinone family), which unlike BIG inhibits GSK3 by non-ATP competition, promoted an effector-like profile instead of a stem-like memory profile in CD8⁺ T cells (including higher frequencies of CD127⁻ T-bet⁺ cells, and LAG-3⁺ PD-1⁺ cells).

TABLE 2
Effect of GSK3 inhibitors on human CD8+ T cells

| | | Fold change in | | | | |
|---|---|---|---|---|---|---|
| | | Optimal | | IFN-γ+ TNF- | | |
| | | dose for | | a+cells | pS6+pAKT+cells | |
| | | CD8 | | (relative | (relative to | % CD127+ |
| GSK3 inhibitor | Class | T cells | | to DMSO) | DMSO) | T-bet− |
| 10Z-Hymenialdisine | Pyrroloazepine | 1.25 | μM | − | − | |
| Aloisine A | Pyrrolopyrazine | 5 | μM | − | − | |
| Alsterpaullone | Benzazepinone | 5 | μM | + + | + + | + |
| AR-A014418 | Thiazole | 10 | μM | − | − | |
| AZD 2858 | Pyrazine | 2 | μM | + + | + + | |
| BIO | Bis-Indole | 3 | μM | + + | − | + |
| (2′Z,3′E)-6-Bromoindirubin-3′- | | | | | | |
| oxime) | | | | | | |
| Bio-acetoxime | Bis-Indole | 3 | μM | + + | − | + |
| CGP 60474 | Phenylaminopyrimidine | 3 | μM | − | − | + |
| CHIR 98014 | Aminopyrimidine | 0.25 | μM | + | + | |
| Flavopiridol hydrochloride hydrate | Flavone | 5 | μM | − | − | + |
| GF 109203X | Bisindolylmaleimide | 3 | μM | − | − | |
| Lithium carbonate | Atom (competition with | 5 | mM | − | + | |
| | Mg2+) | | | | | |
| Me-BIO (negative control of BIO) | Bis-Indole | 3 | μM | − | − | |
| PF-04802367 | 4-carboxamide 5-aryl | 3 | μM | + + | + + | |
| | disubstituted oxazole | | | | | |
| SB-216763 | Arylindolemaleimide | 10 | μM | + | + | |
| SB-415286 | Anilinomaleimide | 5 | μM | − | − | |
| Staurosporine | Bisindolylmaleimide | 10 | nM | − | − | + |
| SU 9516 | Oxindole (indolinone) | 3 | μM | + + | + + | |
| Tideglusib (NP031112) | Thiadiazolidinone | 3 | μM | − | − | |
| TWS119 | Pyrrolopyrimidine | 3 | μM | − | − | |

| | Fold change in | | | | | | | |
|---|---|---|---|---|---|---|---|---|
| | % CD127− | % HLA+ | % LAG3+ | % Naive- | % | % | % | % |
| GSK3 inhibitor | T-bet+ | CD38+ | PD1+ | TSCM | TCM | TTM | TEM | TTE |
| 10Z-Hymenialdisine | | + | + | | + | | | |
| Aloisine A | − | | | | | + | | |
| Alsterpaullone | | − − | − | | − | − | + | − |
| AR-A014418 | − | − − | | − | | | | |
| AZD 2858 | | + | + | | | − | + | |
| BIO | − | − | − | | | | | − |
| (2′Z,3′E)-6-Bromoindirubin-3′- | | | | | | | | |
| oxime) | | | | | | | | |
| Bio-acetoxime | − | | | | | | | − |
| CGP 60474 | | − − | | | − | + | | + |
| CHIR 98014 | | + | + | | + | | | |
| Flavopiridol hydrochloride hydrate | | − − | | − − | − − | | | + |
| GF 109203X | | − − | | − | | + | | |
| Lithium carbonate | + | + + | | | | | | |
| Me-BIO (negative control of BIO) | | | | | | | | |
| PF-04802367 | | | | − | − | | + | |
| SB-216763 | | | | − | − | | | |
| SB-415286 | | + | | | | + | + | + |
| Staurosporine | | − | − | | | + | | |
| SU 9516 | + | + + | | | | | | + |
| Tideglusib (NP031112) | + | + | + | − | + | + | + | |
| TWS119 | | + | | | | | | |

Altogether, these data indicate that GSK3 inhibitors differ in their mechanisms of action, targeted pathways, and effects on CD8⁺ T cells. In addition, the results presented herein open a new avenue of possible applications of GSK3 inhibitors in contexts where either effector-like or memory-like T cells are required.

### DISCUSSION

The inventors report here that reprogramming of HIV-specific CD8⁺ T cells from HIV-1 non-controllers via GSK3 inhibition confers them higher polyfunctionality, survival, expansion capacity, diminished dependency on mTORC1 and glucose, and better capacity to respond to γ-chain cytokines. These characteristics have been previously associated with efficient responses in natural HIV-1 controllers (4-8). Thus, such CD8⁺ T cell reprogramming represents a promising option to enhance the efficacy of cell-based therapies for HIV-1 infection.

The inventors chose targeting of GSK3 based on the implication of this molecule in the modulation of several intracellular pathways that they have identified to be of potential relevance in virus-specific CD8⁺ T cells from HIV-1 (8) and SIV natural controllers (9). BIO and TWS119, the two molecules that were used here, are highly selective inhibitors of GSK3 and induce the activation of the Wnt/β-catenin/TCF-1 pathway (42). Higher TCF-1 expression is a characteristic of virus-specific CD8⁺ T cells in HIV-1 and SIV controllers (7,9,16), and the intensification of its function promotes CD8⁺ T cell stemness, which associates with enhanced viral and tumor control (7,43). It is also well described that stem-like memory TCF-1⁺CD8⁺ T cells have strong antiviral potential and therapeutic benefit (13-15,44,45), related to their longevity and enhanced ability to expand and differentiate into effector subsets (45,46). In addition, GSK3 interacts with the mTOR signaling network (47), and its inhibition promotes the activation of the mTORC2 pathway (48). The mTORC2 pathway promotes the generation of memory CD8⁺ T cells (49) and is preferentially upregulated in HIV-specific CD8⁺ T cells from HICs (8).

According to the effects of GSK3 inhibition, in this study, the inventors show that CD8⁺ T cell reprogramming promoted the expression of TCF-1, a TCM and TSCM phenotype, as well as restrained effector differentiation. Reprogrammed CD8⁺ T cells also regulated proliferation to maintain quiescence, and to limit the consequences of excessive activation (21,39,50). Correspondingly, reprogrammed CD8⁺ T cells shifted their transcriptomic profile towards a program of quiescence and survival, and downregulated several genes associated with anabolic metabolism (51), reflected in lower glucose and lipid consumption, and mitochondrial activity. These data are in line with previous studies showing the critical role of metabolism on CD8⁺ T cell effector versus memory fate (52,53), and the quiescent profile of less differentiated subsets (46,54). Remarkably, both polyclonal and HIV-specific reprogrammed CD8⁺ T cells showed lower activation of mTORC1, and a predilection for mTORC2 pathway activation to sustain their activities. Moreover, reprogrammed HIV-specific CD8⁺ T cells depended less on glucose. Such metabolic plasticity is also found in cells from HICs and may be particularly helpful in hostile metabolic environments such as tissues, with infected cells competing for nutrients (55) and high antigen load and inflammation impacting the survival and function of CD8⁺ T cells (56). The modulation of metabolism and the induction of a stem-like profile are likely related and synergize to improve CD8⁺ T cell antiviral potential, even in settings of persistent or strong antigen stimulation. These properties exhibited by reprogrammed HIV-specific CD8⁺ T cells might contribute to long-term viral control in vivo. Certainly, stem-like SIV-specific CD8⁺ T cells are long-term maintained and are linked to the natural control of infection (9).

The inventors found that reprogramming did not affect all CD8⁺ T cells uniformly. Although BIO induced very similar effects globally on CD8⁺ T cells, reprogramming had the strongest enhancing effect of the activities of HIV-specific CD8⁺ T cells from non-controllers when compared to cells responding to polyclonal stimulation or HCMV peptides. It was previously shown that HCMV and HIV-specific cells from HICs are relatively similar in terms of memory metabolic program (8). In contrast, HIV-specific cells from non-controllers have an effector-like and exhausted profile (8,57,58), and such a biased program can be found even among the TCM population (8). These divergent signatures in cells from controllers and non-controllers could be related to the TCR signaling and priming signals they received in vivo early after infection (9), which may be exacerbated by the persistence of high amounts of antigen and inflammation in non-controller individuals. Thus, it was expected that HIV-specific CD8⁺ T cells derived from non-controllers benefited the most from the effects of reprogramming when compared to better-fitted HCMV-specific cells. Along these lines, these results show that the skewed program of HIV-specific CD8⁺ T cells from non-controllers is at least partially reversible.

These results also show that not all CD8⁺ T cells activities are equally regulated by metabolic pathways. Observations in previous studies suggested that polyfunctionality, and in particular TNF-α production, might depend on the mTORC2 pathway (8). This aspect was confirmed here. It was observed a selective increase in the production of TNF-α by reprogrammed CD8⁺ T cells, which was related to the inhibition of mTORC1, preservation of the mTORC2 pathway, and less dependency on glycolysis. In contrast, IFN-γ and IL-2 production was associated with mTORC1 activation. In keeping with these results, previous studies have demonstrated that TNF-α production is associated with a less-differentiated CD8+ T cell profile (59,60), while the transcription factor NF-χB (regulator of TNF-α expression) is active in stem-like CD8⁺ T cells (45,61), suggesting a role of this signaling pathway as a regulator of this subset. The underlying mechanism of enhanced TNF-α production by reprogrammed CD8⁺ T cells might be related to epigenetic modifications and/or posttranscriptional events (60,62), that deserve further study. Moreover, although further studies are necessary to fully elucidate this aspect, these data suggest that the production of TNF-α in the absence of mTORC1 activity (pS6 expression) could serve as an additional marker of stem-like memory CD8⁺ T cells.

The inventors propose that reprogramming HIV-specific CD8⁺ T cells may be of potential interest for interventions aiming at HIV-1 remission. Two beneficial effects could be expected: first, it was shown here that reprogramming reinvigorates the antiviral potential of HIV-specific CD8⁺ T cells; secondly, the promotion of self-renewal and long-term survival of the cells may improve the therapeutic efficacy of adoptive immunotherapies. Indeed, one of the major caveats in adoptive immunotherapies is the low persistence of transferred cells, which impairs the long-term therapeutic efficacy, as has been observed in HIV-1⁺ individuals receiving autologous, ex vivo-expanded HIV-specific CD8⁺ T cells (63-65). In line with this notion, complete cancer regression and persistence of transferred tumor-reactive CD8⁺ T cells have been associated with a stem-like population capable of self-renewal, expansion, and antitumor responses (66). Of note, CD8⁺ T cell reprogramming could be combined with other interventions to further enhance their antiviral potential. It was previously shown that short IL-15 treatment of HIV-specific CD8⁺ T cells from non-controllers strongly enhanced their capacity to mobilize mitochondrial activities and their capacity to suppress HIV-1 infection of CD4⁺ T cells (8). As reprogrammed CD8⁺ T cells are more responsive to IL-15 in terms of proliferation and maturation of their effector profile, it was hypothesized that a combined approach may have synergistic effects.

In summary, it is shown here that HIV-specific CD8⁺ T cell pharmacologic reprogramming into stem-like cells induces several properties associated with natural control of infection which help to improve the response to immunomodulators. CD8⁺ T cell reprogramming could be used to potentiate the therapeutic efficacy of this cell population in adoptive transfer strategies, as well as the effect of other immunotherapies, in the search for an HIV-1 cure or remission or development of therapeutic vaccines. In addition, the benefits of T cell reprogramming could be extended to the context of other chronic viral infections or tumors where antigen-specific CD8⁺ T cells have a skewed and dysfunctional profile.

### REFERENCES

1. Prlic M, Williams M A, Bevan M J. Requirements for CD8 T-cell priming, memory generation and maintenance. Curr Opin Immunol [Internet]. 2007 June; 19(3):315-9. Available from: https://www.sciencedirect.com/science/article/pii/S0952791507000623
2. Sáez-Cirión A, Pancino G. HIV controllers: a genetically determined or inducible phenotype?Immunol Rev [Internet]. 2013 July; 254(1):281-94. Available from: https://onlinelibrary.wiley.com/doi/full/10.1111/imr.12076
3. Collins D R, Gaiha G D, Walker B D. CD8+ T cells in HIV control, cure and prevention. Nat Rev Immunol [Internet]. 2020; Available from: https://www.nature.com/articles/s41577-020-0274-9
4. Migueles S A, Laborico A C, Shupert W L, Sabbaghian M S, Rabin R, Hallahan C W, et al. HIV-specific CD8+ T cell proliferation is coupled to perforin expression and is maintained in nonprogressors. Nat Immunol [Internet]. 2002 November; 3(11):1061-8. Available from: https://www.nature.com/articles/ni845
5. Yan J, Sabbaj S, Bansal A, Amatya N, Shacka J J, Goepfert P A, et al. HIV-Specific CD8+ T Cells from Elite Controllers Are Primed for Survival. J Virol [Internet]. 2013; 87(9):5170-5181. Available from: https://journals.asm.org/doi/full/10.1128/JVI.02379-12
6. Betts M R, Nason M C, West S M, De Rosa S C, Migueles S A, Abraham J, et al. HIV nonprogressors preferentially maintain highly functional HIV-specific CD8+ T cells. Blood [Internet]. 2006; 107(12):4781-9. Available from: https://ashpublications.org/blood/article/107/12/4781/129277/HIV-nonprogressors-preferentially-maintain-highly
7. Rutishauser R L, Deguit C D T, Hiatt J, Blaeschke F, Roth T L, Wang L, et al. TCF-1 regulates HIV-specific CD8+ T cell expansion capacity. JCI insight [Internet]. 2021 February; 6(3):1-16. Available from: https://insight.jci.org/articles/view/136648
8. Angin M, Volant S, Passaes C, Lecuroux C, Monceaux V, Dillies M-A, et al. Metabolic plasticity of HIV-specific CD8+ T cells is associated with enhanced antiviral potential and natural control of HIV-1 infection. Nat Metab [Internet]. 2019; 1:704-16. Available from: https://www.nature.com/articles/s42255-019-0081-4
9. Passaes C, Millet A, Madelain V, Monceaux V, David A, Versmisse P, et al. Optimal Maturation of the SIV-Specific CD8+ T Cell Response after Primary Infection Is Associated with Natural Control of SIV: ANRS SIC Study. Cell Rep [Internet]. 2020 September; 32(12):108174. Available from: https://www.sciencedirect.com/science/article/pii/S2211124720311633
10. Saez-Cirion A, Lacabaratz C, Lambotte O, Versmisse P, Urrutia A, Boufassa F, et al. HIV controllers exhibit potent CD8 T cell capacity to suppress HIV infection ex vivo and peculiar cytotoxic T lymphocyte activation phenotype. Proc Natl Acad Sci [Internet]. 2007; 104(16):6776-81. Available from: https://www.pnas.org/content/104/16/6776
11. Escobar G, Mangani D, Anderson A C. T cell factor 1: A master regulator of the T cell response in disease. Sci Immunol [Internet]. 2020 November; 5(53):1-12. Available from: https:/immunology.sciencemag.org/content/5/53/eabb9726
12. Gattinoni L, Klebanoff C A, Restifo N P. Paths to stemness: building the ultimate antitumour T cell. Nat Rev Cancer [Internet]. 2012 October; 12(10):671-84. Available from: https://www.nature.com/articles/nrc3322
13. Utzschneider D T, Charmoy M, Chennupati V, Pousse L, Ferreira D P, Calderon-Copete S, et al. T Cell Factor 1-Expressing Memory-like CD8+ T Cells Sustain the Immune Response to Chronic Viral Infections. Immunity [Internet]. 2016 August; 45(2):415-27. Available from: https://www.cell.com/immunity/comments/S1074-7613(16)30294-1
14. Wu T, Ji Y, Moseman E A, Xu H C, Manglani M, Kirby M, et al. The TCF1-Bcl6 axis counteracts type I interferon to repress exhaustion and maintain T cell stemness. Sci Immunol [Internet]. 2016 December; 1(6):1-12. Available from: https:/immunology.sciencemag.org/content/1/6/eaai8593.full
15. Wieland D, Kemming J, Schuch A, Emmerich F, Knolle P, Neumann-Haefelin C, et al. TCF1+ hepatitis C virus-specific CD8+ T cells are maintained after cessation of chronic antigen stimulation. Nat Commun [Internet]. 2017 May; 8:1-13. Available from: https://www.nature.com/articles/ncomms15050
16. Sekine T, Perez-Potti A, Nguyen S, Gorin J-B, Wu V H, Gostick E, et al. TOX is expressed by exhausted and polyfunctional human effector memory CD8+ T cells. Sci Immunol [Internet]. 2020 Jul. 3; 5(49):eaba7918. Available from: http://immunology.sciencemag.org/content/5/49/eaba7918.abstract
17. Kishton R J, Sukumar M, Restifo N P. Metabolic Regulation of T Cell Longevity and Function in Tumor Immunotherapy. Cell Metab [Internet]. 2017 July; 26(1):94-109. Available from: https://www.sciencedirect.com/science/article/pii/S1550413117303583?via %3Dihub
18. Pollizzi K N, Patel C H, Sun I-H, Oh M-H, Waickman A T, Wen J, et al. mTORC1 and mTORC2 selectively regulate CD8+ T cell differentiation. J Clin Invest [Internet]. 2015 May 1; 125(5):2090-108. Available from: https://doi.org/10.1172/JCI77746
19. Araki K, Turner A P, Shaffer V O, Gangappa S, Keller S A, Bachmann M F, et al. mTOR regulates memory CD8 T-cell differentiation. Nature [Internet]. 2009 July; 460(7251):108-12. Available from: https://www.nature.com/articles/nature08155
20. Scholz G, Jandus C, Zhang L, Grandclément C, Lopez-Mejia I C, Soneson C, et al. Modulation of mTOR Signalling Triggers the Formation of Stem Cell-like Memory T Cells. EBioMedicine [Internet]. 2016; 4:50-61. Available from: https://www.sciencedirect.com/science/article/pii/S2352396416300159
21. Gattinoni L, Zhong X—S, Palmer D C, Ji Y, Hinrichs C S, Yu Z, et al. Wnt signaling arrests effector T cell differentiation and generates CD8+ memory stem cells. Nat Med [Internet]. 2009 July; 15(7):808-13. Available from: https://www.nature.com/articles/nm.1982
22. Kaech S M, Cui W. Transcriptional control of effector and memory CD8+ T cell differentiation. Nat Rev Immunol [Internet]. 2012; 12(11):749-61. Available from: https://doi.org/10.1038/nri3307
23. Liu Z, Guo Y, Tang S, Zhou L, Huang C, Cao Y, et al. Cutting Edge: Transcription Factor BCL6 Is Required for the Generation, but Not Maintenance, of Memory CD8+ T Cells in Acute Viral Infection. J Immunol [Internet]. 2019 Jun. 7; 203:ji1900014. Available from: http://www.jimmunol.org/content/early/2019/06/06/jimmunol.1900014.abstract
24. Zhou X, Yu S, Zhao D-M, Harty J T, Badovinac V P, Xue H—H. Differentiation and persistence of memory CD8+ T cells depend on T cell factor 1. Immunity [Internet]. 2010 August; 33(2):229-40. Available from: https://www.sciencedirect.com/science/article/pii/S1074761310002852?via %3Dihub
25. Yao C, Sun H—W, Lacey N E, Ji Y, Moseman E A, Shih H—Y, et al. Single-cell RNA-seq reveals TOX as a key regulator of CD8+ T cell persistence in chronic infection. Nat Immunol [Internet]. 2019; 20(7):890-901. Available from: https://doi.org/10.1038/s41590-019-0403-4
26. Schluns K S, Kieper W C, Jameson S C, Lefrangois L. Interleukin-7 mediates the homeostasis of naïve and memory CD8 T cells in vivo. Nat Immunol [Internet]. 2000 November; 1(5):426-32. Available from: https://www.nature.com/articles/ni1100_426
27. Verma N K, Kelleher D. Not Just an Adhesion Molecule: LFA-1 Contact Tunes the T Lymphocyte Program. J Immunol [Internet]. 2017 August; 199(4):1213-21. Available from: https://www.jimmunol.org/content/199/4/1213.abstract
28. Kurachi M, Barnitz R A, Yosef N, Odorizzi P M, DiIorio M A, Lemieux M E, et al. The transcription factor BATF operates as an essential differentiation checkpoint in early effector CD8+ T cells. Nat Immunol [Internet]. 2014; 15(4):373-83. Available from: https://doi.org/10.1038/ni.2834
29. Sercan Ö, Stoycheva D, Hammerling G J, Arnold B, Schüler T. IFN-γ Receptor Signaling Regulates Memory CD8+ T Cell Differentiation. J Immunol [Internet]. 2010 Mar. 15; 184(6):2855 LP-2862. Available from: http://www.jimmunol.org/content/184/6/2855.abstract
30. McLane L M, Abdel-Hakeem M S, Wherry E J. CD8 T Cell Exhaustion During Chronic Viral Infection and Cancer. Annu Rev Immunol [Internet]. 2019 January; 37:457-95. Available from: https://www.annualreviews.org/doi/abs/10.1146/annurev-immunol-041015-055318
31. Chapman N M, Boothby M R, Chi H. Metabolic coordination of T cell quiescence and activation. Nat Rev Immunol [Internet]. 2020; 20(1):55-70. Available from: https://www.nature.com/articles/s41577-019-0203-y#citeas
32. Salmond R J. mTOR Regulation of Glycolytic Metabolism in T Cells [Internet]. Vol. 6, Frontiers in Cell and Developmental Biology. 2018. p. 122. Available from: https://www.frontiersin.org/article/10.3389/fcell.2018.00122
33. Michalek R D, Gerriets V A, Nichols A G, Inoue M, Kazmin D, Chang C—Y, et al. Estrogen-related receptor-α is a metabolic regulator of effector T-cell activation and differentiation. Proc Natl Acad Sci USA [Internet]. 2011/10/31. 2011 Nov. 8; 108(45):18348-53. Available from: https://pubmed.ncbi.nlm.nih.gov/22042850
34. Johnson M O, Wolf M M, Madden M Z, Andrejeva G, Sugiura A, Contreras D C, et al. Distinct Regulation of Th17 and Th1 Cell Differentiation by Glutaminase-Dependent Metabolism. Cell [Internet]. 2018/11/01. 2018 Dec. 13; 175(7):1780-1795.e19. Available from: https://pubmed.ncbi.nlm.nih.gov/30392958
35. Rao E, Zhang Y, Zhu G, Hao J, Persson X-MT, Egilmez N K, et al. Deficiency of AMPK in CD8+ T cells suppresses their anti-tumor function by inducing protein phosphatase-mediated cell death. Oncotarget [Internet]. 2015 Apr. 10; 6(10):7944-58. Available from: https://pubmed.ncbi.nlm.nih.gov/25760243
36. Reina-Campos M, Scharping N E, Goldrath A W. CD8+ T cell metabolism in infection and cancer. Nat Rev Immunol [Internet]. 2021; 21(11):718-38. Available from: https://doi.org/10.1038/s41577-021-00537-8
37. Chowdhury F Z, Ouyang Z, Buzon M, Walker B D, Lichterfeld M, Yu X G. Metabolic pathway activation distinguishes transcriptional signatures of CD8+ T cells from HIV-1 elite controllers. AIDS [Internet]. 2018 November; 32(18):2669-77. Available from: https://journals.lww.com/aidsonline/pages/articleviewer.aspx?year=2018&issue=11280&article=00002&type=Fulltext
38. Liebmann M, Hucke S, Koch K, Eschborn M, Ghelman J, Chasan A I, et al. Nur77 serves as a molecular brake of the metabolic switch during T cell activation to restrict autoimmunity. Proc Natl Acad Sci USA [Internet]. 2018 August; 115(34):E8017-26. Available from: https://www.pnas.org/content/115/34/E8017.long
39. Johnnidis J B, Muroyama Y, Ngiow S F, Chen Z, Manne S, Cai Z, et al. Inhibitory signaling sustains a distinct early memory CD8+ T cell precursor that is resistant to DNA damage. Sci Immunol [Internet]. 2021 Jan. 15; 6(55):eabe3702. Available from: http://immunology.sciencemag.org/content/6/55/eabe3702.abstract
40. Powell J D, Pollizzi K N, Heikamp E B, Horton M R. Regulation of immune responses by mTOR. Annu Rev Immunol [Internet]. 2012; 30:39-68. Available from: https://www.annualreviews.org/doi/full/10.1146/annurev-immunol-020711-075024
41. Boyman O, Purton J F, Surh C D, Sprent J. Cytokines and T-cell homeostasis. Curr Opin Immunol [Internet]. 2007 June; 19(3):320-6. Available from: https://www.sciencedirect.com/science/article/pii/S0952791507000672?via %3Dihub
42. Meijer L, Skaltsounis A-L, Magiatis P, Polychronopoulos P, Knockaert M, Leost M, et al. GSK3-selective inhibitors derived from Tyrian purple indirubins. Chem Biol [Internet]. 2003 December; 10(12):1255-66. Available from: https://www.sciencedirect.com/science/article/pii/S1074552103002552
43. Shan Q, Hu S, Chen X, Danahy D B, Badovinac V P, Zang C, et al. Ectopic Tcf1 expression instills a stem-like program in exhausted CD8+ T cells to enhance viral and tumor immunity. Cell Mol Immunol [Internet]. 2021; 18(5):1262-77. Available from: https://doi.org/10.1038/s41423-020-0436-5
44. Im S J, Hashimoto M, Gerner M Y, Lee J, Kissick H T, Burger M C, et al. Defining CD8+ T cells that provide the proliferative burst after PD-1 therapy. Nature [Internet]. 2016; 537:417-21. Available from: https://www.nature.com/articles/nature19330
45. Miller B C, Sen D R, Al Abosy R, Bi K, Virkud Y V, LaFleur M W, et al. Subsets of exhausted CD8+ T cells differentially mediate tumor control and respond to checkpoint blockade. Nat Immunol [Internet]. 2019; 20(3):326-36. Available from: https://doi.org/10.1038/s41590-019-0312-6
46. Galletti G, De Simone G, Mazza E M C, Puccio S, Mezzanotte C, Bi T M, et al. Two subsets of stem-like CD8+ memory T cell progenitors with distinct fate commitments in humans. Nat Immunol [Internet]. 2020 December; 21(12):1552-62. Available from: https://www.nature.com/articles/s41590-020-0791-5
47. Hermida M A, Dinesh Kumar J, Leslie N R. GSK3 and its interactions with the PI3K/AKT/mTOR signalling network. Adv Biol Regul [Internet]. 2017; 65:5-15. Available from: https://www.sciencedirect.com/science/article/pii/S2212492617301240
48. Koo J, Wu X, Mao Z, Khuri F R, Sun S—Y. Rictor Undergoes Glycogen Synthase Kinase 3 (GSK3)-dependent, FBXW7-mediated Ubiquitination and Proteasomal Degradation*. J Biol Chem [Internet]. 2015; 290(22):14120-9. Available from: https://www.sciencedirect.com/science/article/pii/S0021925818809176
49. Huang H, Long L, Zhou P, Chapman N M, Chi H. mTOR signaling at the crossroads of environmental signals and T-cell fate decisions. Immunol Rev [Internet]. 2020/03/25. 2020 May; 295(1):15-38. Available from: https://pubmed.ncbi.nlm.nih.gov/32212344
50. Lin W—HW, Nish S A, Yen B, Chen Y—H, Adams W C, Kratchmarov R, et al. CD8+ T Lymphocyte Self-Renewal during Effector Cell Determination. Cell Rep [Internet]. 2016 November; 17(7):1773-82. Available from: https://www.sciencedirect.com/science/article/pii/S2211124716314371
51. Zhang L, Romero P. Metabolic Control of CD8+ T Cell Fate Decisions and Antitumor Immunity. Trends Mol Med [Internet]. 2018 January; 24(1):30-48. Available from: https://www.cell.com/trends/molecular-medicine/fulltext/S1471-4914(17)30205-8
52. Pearce E L, Walsh M C, Cejas P J, Harms G M, Shen H, Wang L-S, et al. Enhancing CD8 T-cell memory by modulating fatty acid metabolism. Nature [Internet]. 2009; 460(7251):103-7. Available from: https://doi.org/10.1038/nature08097
53. Sukumar M, Liu J, Ji Y, Subramanian M, Crompton J G, Yu Z, et al. Inhibiting glycolytic metabolism enhances CD8+ T cell memory and antitumor function. J Clin Invest [Internet]. 2013/09/16. 2013 October; 123(10):4479-88. Available from: https://pubmed.ncbi.nlm.nih.gov/24091329
54. Crompton J G, Narayanan M, Cuddapah S, Roychoudhuri R, Ji Y, Yang W, et al. Lineage relationship of CD8+ T cell subsets is revealed by progressive changes in the epigenetic landscape. Cell Mol Immunol [Internet]. 2016; 13(4):502-13. Available from: https://doi.org/10.1038/cmi.2015.32
55. Valle-Casuso J C, Angin M, Volant S, Passaes C, Monceaux V, Mikhailova A, et al. Cellular Metabolism Is a Major Determinant of HIV-1 Reservoir Seeding in CD4(+) T Cells and Offers an Opportunity to Tackle Infection. Cell Metab [Internet]. 2019 March; 29(3):611-626.e5. Available from: https://www.sciencedirect.com/science/article/pii/S1550413118307344?via %3Dihub
56. Lim A R, Rathmell W K, Rathmell J C. The tumor microenvironment as a metabolic barrier to effector T cells and immunotherapy. Elife [Internet]. 2020 May 5; 9:e55185. Available from: https://pubmed.ncbi.nlm.nih.gov/32367803
57. Day C L, Kaufmann D E, Kiepiela P, Brown J A, Moodley E S, Reddy S, et al. PD-1 expression on HIV-specific T cells is associated with T-cell exhaustion and disease progression. Nature [Internet]. 2006; 443(7109):350-4. Available from: https://www.nature.com/articles/nature05115
58. Trautmann L, Janbazian L, Chomont N, Said E A, Gimmig S, Bessette B, et al. Upregulation of PD-1 expression on HIV-specific CD8+ T cells leads to reversible immune dysfunction. Nat Med [Internet]. 2006; 12(10):1198-202. Available from: https://www.nature.com/articles/nm1482
59. Brehm M A, Daniels K A, Welsh R M. Rapid production of TNF-alpha following TCR engagement of naive CD8 T cells. J Immunol [Internet]. 2005 October; 175(8):5043-9. Available from: https://www.jimmunol.org/content/175/8/5043
60. Denton A E, Russ B E, Doherty P C, Rao S, Turner S J. Differentiation-dependent functional and epigenetic landscapes for cytokine genes in virus-specific CD8+ T cells. Proc Natl Acad Sci USA [Internet]. 2011 September; 108(37):15306-11. Available from: https://www.pnas.org/content/108/37/15306
61. Yao C, Lou G, Sun H—W, Zhu Z, Sun Y, Chen Z, et al. BACH2 enforces the transcriptional and epigenetic programs of stem-like CD8+ T cells. Nat Immunol [Internet]. 2021; 22(3):370-80. Available from: https://doi.org/10.1038/s41590-021-00868-7
62. Salerno F, Paolini N A, Stark R, von Lindern M, Wolkers M C. Distinct PKC-mediated posttranscriptional events set cytokine production kinetics in CD8+ T cells. Proc Natl Acad Sci [Internet]. 2017 Sep. 5; 114(36):9677 LP-9682. Available from: http://www.pnas.org/content/114/36/9677.abstract
63. Lieberman J, Skolnik P R, Parkerson G R 3rd, Fabry J A, Landry B, Bethel J, et al. Safety of autologous, ex vivo-expanded human immunodeficiency virus (HIV)-specific cytotoxic T-lymphocyte infusion in HIV-infected patients. Blood [Internet]. 1997 September; 90(6):2196-206. Available from: https://ashpublications.org/blood/article/90/6/2196/174718/Safety-of-Autologous-Ex-Vivo-Expanded-Human
64. Tan R, Xu X, Ogg G S, Hansasuta P, Dong T, Rostron T, et al. Rapid death of adoptively transferred T cells in acquired immunodeficiency syndrome. Blood [Internet]. 1999 March; 93(5):1506-10. Available from: https://ashpublications.org/blood/article/93/5/1506/247827/Rapid-Death-of-Adoptively-Transferred-T-Cells-in
65. Brodie S J, Lewinsohn D A, Patterson B K, Jiyamapa D, Krieger J, Corey L, et al. In vivo migration and function of transferred HIV-1-specific cytotoxic T cells. Nat Med [Internet]. 1999 January; 5(1):34-41. Available from: https://www.nature.com/articles/nm0199_34
66. Krishna S, Lowery F J, Copeland A R, Bahadiroglu E, Mukherjee R, Jia L, et al. Stem-like CD8 T cells mediate response of adoptive cell immunotherapy against human cancer. Science (80-) [Internet]. 2020 Dec. 11; 370(6522):1328 LP-1334. Available from: http://science.sciencemag.org/content/370/6522/1328.abstract.

1. A method for reprogramming CD8+ T cells, comprising administering a glycogen synthase kinase-3 (GSK3) inhibitor, in vitro, to a population of human CD8+ T cells, wherein the GSK3 inhibitor induces reprogrammed CD8+ T cells having enhanced stemness and functional properties or a modulation of effector cells, compared to non-reprogrammed CD8+ T cells.

2. The method according to claim 1, wherein the CD8+ T cells are from peripheral blood.

3. The method according to claim 1 or claim 2, wherein the CD8+ T cells of the population are resting.

4. The method according to any one of claims 1 to 3, wherein the GSK3 inhibitor is selected from the group consisting of: small metal cations; small-molecule ATP-competitive inhibitors; small-molecule non-ATP competitive inhibitors; substrate-competitive peptide inhibitors; siRNAs; miRNAs, ribozymes; epigenome editing enzyme complexes; antagonist peptides; antagonist antibodies; antagonist aptamers, and combinations thereof.

5. The method according to any one of claims 1 to 4, wherein the GSK3 inhibitor is selected from the group consisting of: Anilinomaleimides, ions, Pyrroloazepines, Flavones, Benzazepinones, Bis-Indoles, in particular Indirubins, Pyrrolopyrazines, Aminopyrimidines, Oxindoles, Thiazoles, Bisindolylmaleimides, Phenylaminopyrimidines, Pyrrolopyrimidines, Pyrazines, Thiadiazolidinones, Oxazole-carboxamides, and combinations thereof.

6. The method according to claim 5, wherein the GSK3 inhibitor is an indirubin of formula (I) or a pharmaceutically acceptable salt thereof: 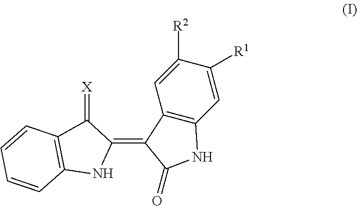
wherein
R¹ is a halogen, or a vinyl group (—CH═CH₂);
R² is selected from the group consisting of H, halogen, amino, nitro and C₁₋₅ alkyl; and
X is O or N—OR³, wherein R³ is selected from the group consisting of H, -(A)-R⁴, —C(O)R⁵ and —C(O)N(R⁶, R⁷), with
A being a non-substituted C₁₋₅ alkylene group or a C₁₋₅ alkylene group substituted by one or several A¹ groups, A¹ being halogen, OH, OR⁸ or NH₂, R⁸ being a C₁₋₅ alkyl;
R⁴ being selected from the group consisting of H, halogen, OH and —N(R⁶, R⁷);
R⁵ being a C₁₋₅ alkyl;
R⁶ and R⁷, identical or different, being a non-substituted C₁₋₅ alkyl or a C₁₋₅ alkyl substituted by A¹ such as above defined, or R⁶ and R⁷ are part of a cycle with 5 or 6 elements, optionally comprising another heteroatom such as O or N.

7. The method according to claim 5 or 6, wherein the GSK3 inhibitor is selected from the group consisting of: SB-415286, Lithium carbonate, 10Z-Hymenialdisine, Flavopiridol hydrochloride hydrate, Alsterpaullone, 6-bromoindirubin-3′-oxime and 6-bromoindirubin-3′-acetoxime, Aloisine A, CHIR 98014, SU9516, AR-A014418, Staurosporine, GF 109203X, CGP 60474, TWS119, AZD2858, NP031112, SB-216763, PF-04802367, functional analogs or derivatives thereof, and combinations thereof.

8. The method according to claim 6 or 7, wherein the GSK3 inhibitor is 6-bromoindirubin-3′-oxime 6-bromoindirubin-3′-acetoxime, functional analog or derivative thereof, or combinations thereof.

9. The method according to any one of claims 1 to 8, wherein the GSK3 inhibitor for CD8+ T cell reprogramming induces CD8+ T cells having enhanced stemness and functional properties and is selected from indirubins, in particular indirubin of formula (I); preferably selected from the group consisting of: 6-bromoindirubin-3′-oxime, 6-bromoindirubin-3′-acetoxime, 6-bromo-indirubin, 6-bromo-indirubin-3′methoxime, 6-chloro-indirubin, 6-chloro-indirubin-3′-oxime, 6-chloro-indirubin-3′-acetoxime, 6-iodo-indirubin, 6-iodo-indirubin-3′-oxime, 6-iodo-indirubin-3′-acetoxime, 6-vinylindirubin-3′-oxime, 6-vinylindirubin-3′-acetoxime, 6-fluoroindirubin-3′-oxime, 6-fluoroindirubin-3′-acetoxime, 6-bromo-5-methylindirubin, 6-bromo-5-methylindirubin-3′-oxime, 6-bromo-5-methylindirubin-3′-acetoxime, 5,6-dichloroindirubin, 5,6-dichloroindirubin-3′-oxime, 5,6-dichloroindirubin-3′-acetoxime, 6-bromo-5-nitroindirubin, 6-bromo-5-nitroindirubin-3′-oxime, 6-bromo-5-nitroindirubin-3′-acetoxime, 6-bromo-5-aminoindirubin, 6-bromo-5-aminoindirubin-3′-oxime, 5,6-dibromoindirubin, 6-bromoindirubin-3′-[O-(2-bromoethyl)-oxime], 6-bromoindirubin-3′-[O-(2-hydroxyethyl)-oxime], 6-bromoindirubin-3′-[O-(2,3-dihydroxypropyl)-oxime], 6-bromoindirubin-3′-[O—(N,N-diethylcarbamyl)-oxime], 6-bromoindirubin-3′-[O-(2-dimethylaminoethyl)-oxime], 6-bromoindirubin-3′-[O-(2-diethylaminoethyl)-oxime], 6-bromoindirubin-3′-[O-(2-pyrrolidin-1-ylethyl)oxime], 6-bromoindirubin-3′-[O-(2-morpholin-1-ylethyl)oxime], 6-bromoindirubin-3′-[O-(2-(N,N-(2-hydroxyethyl)aminoethyl)oxime], 6-bromoindirubin-3′-(O-{2-[N-methyl, N-(2,3-dihydroxypropyl)amino]ethyl}oxime], 6-bromoindirubin-3′-[O-(2-piperazine-1-ylethyl)oxime], 6-bromoindirubin-3′-(0-[2-(4-methyl-piperazin-1-yl)ethyl]oxime), 6-bromoindirubin-3′-(O-{2-[4-(2-hydroxyethyl)piperazin-1-yl]ethyl}oxime), 6-bromoindirubin-3′-(O-{2-[4-(2-methoxyethyl)piperazin-1-yl]ethyl}oxime), 6-bromoindirubin-3′-[O-(2-(4-[2-(2-hydroxyethoxy)-ethyl]piperazin-1-yl)ethyl)oxime], 6-bromoindirubin-3′-[O-(2-dimethylaminoethyl)oxime]hydrochloride, 6-bromoindirubin-3′-[O-(2-diethylaminoethyl)oxime]hydrochloride, 6-bromoindirubin-3′-[O-(2-pyrrolidin-1-ylethyl)oxime]hydrochloride, 6-bromoindirubin-3′-[O-(2-morpholin-1-ylethyl)oxime]hydrochloride, 6-bromoindirubin-3′-[O-(2-(N,N-(2-hydroxyethyl)aminoethyl)oxime], 6-bromoindirubin-3′-(O-{2-[N-methyl, N-(2,3-dihydroxypropyl)amino]ethyl}oxime]hydrochloride, 6-bromoindirubin-3′-[O-(2-piperazine-1-ylethyl)oxime]dihydrochloride, 6-bromoindirubin-3′-{0-[2-(4-methylpiperazin-1-yl)ethyl]oxime}dihydrochloride, 6-bromoindirubin-3′-(O-{2-[4-(2-hydroxyethyl)piperazin-1-yl]ethyl}oxime)dihydrochloride, 6-bromoindirubin-3′-(O-{2-[4-(2-methoxyethyl)piperazin-1-yl]ethyl}oxime)dihydrochloride and 6-bromoindirubin-3′-[O-(2-{4-[2-(2-hydroxyethoxy)-ethyl]piperazin-1-yl}ethyl)oxime]dihydrochloride; preferably 6-bromoindirubin-3′-oxime and 6-bromoindirubin-3′-acetoxime.

10. The method according to any one of claims 1 to 8, wherein the GSK3 inhibitor for CD8+ T cell reprogramming enhances effector CD8⁺ T cells and is selected from the group consisting of: Anilinomaleimides; Benzazepinones; Aminopyrimidines, Oxindoles; Pyrazines and Oxazole-carboxamides; preferably selected from the group consisting of: Alsterpaullone, CHIR 98014, SU9516, AZD2858, SB-216763, PF-04802367, functional analogs or derivatives thereof, and combinations thereof.

11. The method according to any one of claims 1 to 8, wherein the GSK3 inhibitor for CD8+ T cell reprogramming represses effector CD8⁺ T cells and is selected from the group consisting of: Anilinomaleimides and Bisindolylmaleimides; Pyrroloazepines; Flavones; Pyrrolopyrazines; Pyrrolopyrimidines and Phenylaminopyrimidines; Thiazoles; and Thiadiazolidinones; preferably is selected from the group consisting of: SB-415286, 10Z-Hymenialdisine, Flavopiridol hydrochloride hydrate, Aloisine A, AR-A014418, Staurosporine, GF 109203X, CGP 60474, TWS119, NP031112, functional analogs or derivatives thereof, and combinations thereof.

12. The method according to any one of claims 1 to 11, wherein the GSK3 inhibitor is at a concentration of from 1 to 5 μM.

13. The method according to any one of claims 1 to 12, wherein the GSK3 inhibitor is contacted with the population of CD8+ T cells for 6 to 16 hours.

14. The method according to any one of claims 1 to 13, wherein the reprogrammed CD8+ T cells have enhanced survival, polyfunctionality, proliferation capacity, metabolic plasticity, response to antigen, response to γ-chain cytokines, response to immune check-point modulators, cytotoxic effect, and/or less mTORC1-dependency, compared to non-reprogrammed CD8+ T cells.

15. The method according to claim 14, wherein the reprogrammed CD8+ T cells have enhanced expression level of TCF-1 in the absence of further stimulation; preferably further having enhanced expression levels of CCR7 and CD27 in the absence of further stimulation.

16. The method according to claim 14 or 15, wherein the reprogrammed CD8+ T cells have enhanced expression levels of TCF-1, CD127 and TNF-α in response to T-cell receptor (TCR) stimulation: more preferably, further having enhanced expression levels of CD122 and/or CD215 in response to TCR stimulation.

17. The method according to any one of claims 14 to 16, wherein the reprogrammed CD8+ T cells have enhanced proliferation in response to stimulation with IL-7 and/or IL-15.

18. The method according to any one of claims 14 to 17, wherein the reprogrammed CD8+ T cells have an enhanced response to antigen, characterized by: (i) a higher frequency of antigen-specific CD8+ T cells that are enriched in Stem cell memory and Central memory CD8+ T cells; (ii) higher frequency of antigen-specific CD8+ T cells producing cytokines such as TNF-α and/or (iii) antigen-specific CD8+ T cells having higher survival.

19. The method according to any one of claims 14 to 18, wherein the reprogrammed CD8+ T cells have an enhanced polyfunctionality characterized by a higher frequency of TNF-α+ and IFN-γ+CD8+ T cells; preferably a higher frequency of TNF-α+, IFN-γ+ and IL-2+CD8+ T cells; more preferably a higher frequency of TNF-α+, IFN-γ+, IL-2+ and granzyme B+CD8+ T cells.

20. The method according to any one of claims 14 to 19, wherein the reprogrammed CD8+ T cells have less mTORC1-dependency in response to TCR stimulation, characterized by a lower frequency of pS6+ cells and/or higher frequency of pS6⁻pAKT⁺CD8+ T cells.

21. The method according to any one of claims 14 to 20, wherein the reprogrammed CD8+ T cells have an enhanced metabolic plasticity characterized by maintenance of a higher production of TNF-α despite glucose deprivation.

22. The method according to any one of claims 1 to 21, wherein the reprogrammed CD8+ T cells comprise a higher proportion of memory CD8+ T cells with stemness compared to the non-reprogrammed CD8+ T cells.

23. The method according to claim 22, wherein the proportion of memory CD8+ T cells with stemness is increased by 2-fold to up to 10-fold.

24. The method according to claim 22 or claim 23, wherein the reprogrammed memory CD8+ T cells with stemness have a higher frequency of antigen-specific CD8+ T cells and an enhanced proliferation in response to IL-7 and/or IL-15, compared to non-reprogrammed memory CD8+ T cells with stemness.

25. The method according to any one of claims 1 to 14, wherein the reprogrammed CD8+ T cells have a reduced polyfunctionality in response to TCR stimulation characterized by a lower frequency of TNF-α+ and IFN-γ+CD8+ T cells compared to non-reprogrammed CD8+ T cells.

26. The method according to any one of claims 1 to 14 and 25, wherein the reprogrammed CD8+ T cells have a high activation of mTORC1 and mTORC2 pathways in response to TCR stimulation, characterized by a higher frequency of pS6⁺pAKT⁺CD8+ T cells compared to non-reprogrammed CD8+ T cells.

27. The method according to any one of claims 1 to 14 and 25, wherein the reprogrammed CD8+ T cells have a low activation of mTORC1 and mTORC2 pathways in response to TCR stimulation, characterized by a lower frequency of pS6⁺pAKT⁺CD8+ T cells compared to non-reprogrammed CD8+ T cells.

28. The method according to claim 25 or 27, wherein the reprogrammed CD8+ T cells have: (i) a reduced polyfunctionality in response to TCR stimulation characterized by a lower frequency of TNF-α+ and IFN-γ+CD8+ T cells and (ii) a low activation of mTORC1 and mTORC2 pathways in response to TCR stimulation, characterized by a lower frequency of pS6⁺pAKT⁺CD8+ T cells, compared to non-reprogrammed CD8⁺ T cells.

29. The method according to claim 26, wherein the reprogrammed CD8+ T cells have: (i) an enhanced polyfunctionality in response to TCR stimulation characterized by a higher frequency of TNF-α+ and IFN-γ+CD8+ T cells and (ii) a high activation of mTORC1 and mTORC2 pathways in response to TCR stimulation, characterized by a higher frequency of pS6⁺pAKT⁺CD8+ T cells, compared to non-reprogrammed CD8+ T cells.

30. The method according to claim 27, wherein the reprogrammed CD8+ T cells have: (i) an enhanced polyfunctionality in response to TCR stimulation characterized by a higher frequency of TNF-α+ and IFN-γ+CD8+ T cells and (ii) a low activation of mTORC1 and mTORC2 pathways in response to TCR stimulation, characterized by a lower frequency of pS6⁺pAKT⁺CD8+ T cells, compared to non-reprogrammed CD8⁺ T cells.

31. The method according to any one of claims 1 to 14, 26 and 29, wherein the reprogrammed CD8+ T cells comprise a higher frequency of effector CD8+ T cells, in particular CD127− T-bet+; LAG-3+PD-1+; HLA-DR+CD38+; Effector memory (TEM and/or Terminal effector (TTE) CD8+ T cells, compared to the non-reprogrammed CD8+ T cells.

32. The method according to any one of claims 1 to 14, 25, 27-28, wherein the reprogrammed CD8+ T cells comprise a lower frequency of effector CD8+ T cells, in particular CD127− T-bet+; LAG-3+PD-1+; HLA-DR+CD38+; Effector memory (TEM and/or Terminal effector (TTE) CD8+ T cells, compared to the non-reprogrammed CD8+ T cells.

33. The method according to any one of claims 1 to 32, wherein the reprogrammed CD8+ T cells have enhanced antiviral or antitumoral effects.

34. The method according to any one of claims 1 to 33, wherein the reprogrammed CD8+ T cells have enhanced efficacy for adoptive T cell therapy.

35. The method according to any one of claims 1 to 34, further comprising isolating, stimulating, expanding, engineering, and/or activating the reprogrammed CD8+ T cells.

36. The method according to claim 35, further comprising stimulating the reprogrammed CD8+ T cells with IL-15.

37. The method according to any one of claims 1 to 36, further comprising administering the reprogrammed CD8+ T cells to a subject.

38. The method according to claim 37, wherein the reprogrammed CD8+ T cells are autologous or allogenic.

39. The method according to any one of claims 1 to 38, wherein the subject has an infectious disease or cancer.

40. A pharmaceutical composition comprising an effective amount of the reprogrammed CD8+ T cells produced by the method according to any one of claims 1 to 39, and a pharmaceutically acceptable vehicle and/or carrier.

41. A method for treatment of a patient in need thereof, comprising:
Reprogramming autologous or allogenic CD8+ T cells according to the method of any one of claims 1 to 39; and
Administering the reprogrammed CD8+ T cells to the patient.

42. The method according to claim 41, wherein the reprogrammed CD8+ T cells are expanded before administering to the subject.

43. The method according to claim 41 or 42, wherein the reprogrammed CD8+ T cells are CAR-T cells.

44. The method according to any one of claims 41 to 43, which is for treatment of an infectious disease or cancer.

45. The method according to claim 44, wherein the infectious disease is a viral disease.

46. The method according to claim 45, wherein the viral disease is selected from the group consisting of: HIV/AIDS, Hepatitis B, Hepatitis C, HTLV and CMV infections.

47. The method according to any one of claims 41 to 46, further comprising administering an anticancer therapy, anti-infectious therapy and/or immunotherapy to the patient.

48. The method according to claim 47, wherein the immunotherapy comprises, therapeutic cytokines, immune checkpoint inhibitors and/or co-stimulatory antibodies.

49. The method according to claim 47, wherein the anti-infectious therapy comprises antiretroviral therapy.

50. The method according to claim 49, wherein said antiretroviral therapy is combined antiretroviral therapy.

51. A screening method for inducers of reprogramming of CD8+ T cells comprising:
(a) administering a GSK3 inhibitor to a population of CD8+ T cells in vitro and
(b₁) measuring the level of expression of TCF-1, CCR7, CD27, and/or CD127 and/or the level of activation of mTORC in the population of CD8+ T cells; or
(b₂) measuring: (i) the level of expression of: CD127, T-bet, CD38, HLA-DR, PD-1 and/or LAG-3; (ii) the level of activation of mTORC and/or (iii) the polyfunctionality in the population of CD8+ T cell.

52. The method according to claim 51, wherein the level of expression of TCF-1, CCR7, CD27, and/or CD127 is increased in the population of CD8+ T cells by the GSK3 inhibitor.

53. The method according to claim 51, wherein the level of activation of mTORC1 is decreased and/or the level of activation of mTORC2 is increased in the population of CD8+ T cells by the GSK3 inhibitor.

54. The method according to claim 51, wherein the frequency of CD127− T-bet+; LAG-3+PD-1+; and/or HLA-DR+CD38+ cells is decreased in the population of CD8+ T cells by the GSK3 inhibitor.

55. The method according to claim 51, wherein the frequency of CD127− T-bet+; LAG-3+PD-1+; and/or HLA-DR+CD38+ cells is increased in the population of CD8+ T cells by the GSK3 inhibitor.

56. The method according to claim 51, wherein the level of activation of mTORC1 and mTORC2 in response to TCR stimulation is decreased in the population of CD8+ T cells by the GSK3 inhibitor.

57. The method according to claim 51, wherein the polyfunctionality in response to TCR stimulation is increased in the population of CD8+ T cells by the GSK3 inhibitor.

58. The method according to claim 51, wherein the polyfunctionality in response to TCR stimulation is decreased in the population of CD8+ T cells by the GSK3 inhibitor.

59. The method according to any one of claims 51 to 58, further comprising isolating, stimulating, expanding, engineering, and/or activating CD8+ T cells of the population.

60. The method according to any one of claims 51 to 59, further comprising administering CD8+ T cells of the population to a subject.

61. The method according to any one of claims 51 to 60, wherein the GSK3 inhibitor is selected from the group consisting of: Anilinomaleimides, ions, Pyrroloazepines, Flavones, Benzazepinones, Bis-Indoles, in particular indirubins, Pyrrolopyrazines, Aminopyrimidines, Oxindoles, Thiazoles, Bisindolylmaleimides, Phenylaminopyrimidines, Pyrrolopyrimidines, Pyrazines, Thiadiazolidinones, Oxazole-carboxamides, and combinations thereof.

62. The method according to claim 61, wherein the GSK3 inhibitor is selected from the group consisting of: SB-415286, Lithium carbonate, 10Z-Hymenialdisine, Flavopiridol hydrochloride hydrate, Alsterpaullone, 6-bromoindirubin-3′-oxime and 6-bromoindirubin-3′-acetoxime, Aloisine A, CHIR 98014, SU9516, AR-A014418, Staurosporine, GF 109203X, CGP 60474, TWS119, AZD2858, NP031112, SB-216763, PF-04802367, functional analogs or derivatives thereof, and combinations thereof.
